(19) United States
(12) Patent Application Publication
(10) Pub. No.: US 2011/0020814 A1
(43) Pub. Date: Jan. 27, 2011

(54) METHODS AND COMPOSITIONS FOR SELECTION OF STEM CELLS

(75) Inventors: JOHN DIMOS, SOUTH SAN FRANCISCO, CA (US); KENNETH J. SEIDENMAN, SOUTH SAN FRANCISCO, CA (US)

Correspondence Address: WILSON, SONSINI, GOODRICH & ROSATI, 650 PAGE MILL ROAD, PALO ALTO, CA, 94304-1050, US

(73) Assignee: IPIERIAN, INC., SOUTH SAN FRANCISCO, CA (US)

(21) Appl. No.: 12/794,630

(22) Filed: Jun. 4, 2010

Related U.S. Application Data
(60) Provisional application No. 61/184,733, filed on Jun. 5, 2009.

(51) Int. Cl. C12Q 1/68; G01N 33/53; C12N 5/10; C07H 21/04

(52) U.S. Cl. 435/6; 435/7.21; 435/325; 536/23.1; 536/23.2

(57) ABSTRACT

Described herein are methods and compositions for selection of pluripotent stem cells from a population of mammalian cells (e.g., human) undergoing a process that induces some cells within the population to become pluripotent.

### CROSS-REFERENCE

This application claims the benefit of U.S. Provisional Application No. 61/184,733, filed Jun. 5, 2009, which application is incorporated herein by reference in its entirety.

### BACKGROUND OF THE INVENTION

The induction of autologous pluripotent stem (iPS) cells from a variety of cell types holds much promise for the future of drug development, personalized medicine, and regenerative medicine. However, to date, the induction and isolation of iPS cells is an inefficient, time- and resource-intensive process that hampers efficient application of this technology.

### SUMMARY OF THE INVENTION

This disclosure encompasses methods and related compositions for identifying or selecting induced pluripotent stem (iPS) cells from a population of mammalian cells undergoing induction of pluripotency. Further described herein are methods and compositions for maintaining the pluripotency of populations of iPS cells, and maintaining the differentiated state of cells generated by differentiating iPS cells.

Accordingly, in one aspect provided herein is a method for identifying induced pluripotent stem cells, comprising introducing into the genome of a plurality of mammalian cells an expression vector encoding a selection marker operably linked to a promoter that drives expression of the encoded selection marker in the plurality of mammalian cells and is capable of undergoing transcriptional silencing in a pluripotent stem cell, and culturing the plurality of mammalian cells for a period after the introducing step, wherein
(i) the plurality of mammalian cells comprise at least one exogenous nucleic acid encoding at least one induction factor;
(ii) the plurality of mammalian cells comprise at least one exogenous induction factor;
(iii) the plurality of mammalian cells are contacted with at least one inducing agent; or
(iv) any combination of (i)-(iii); and assaying activity or expression of the encoded selection marker from the introduced expression vector, wherein reduced or absent expression of the selection marker in one or more of the plurality of mammalian cells after the period indicates that the one or more mammalian cells are induced pluripotent stem cells.

In some embodiment of the just-mentioned method, the promoter comprises one or more retroviral silencing elements. In some exemplary embodiments, such retroviral silencing elements comprise the MoMLV LTR negative control region (NCR), direct repeat (DR) enhancer, or primer binding site (PBS). In some embodiments, the promoter comprises one or more retroviral silencing elements from the MoMLV LTR promoter. In one embodiment, the promoter comprises the MoMLV LTR promoter.

In some embodiments, the introducing step comprises transducing the cell with a recombinant retrovirus comprising the expression vector for expression of the selection marker.

In some embodiments, the at least one encoded induction factor or the at least one induction factor comprise, Oct4, Sox2, Klf4, c-Myc, Nanog, Lin-28, or functional homologs thereof. In some embodiments, the at least one nucleic acid encodes three exogenous induction factors. In one embodiment, the three encoded exogenous induction factors are Oct4, Sox2, and Klf4. In some embodiments, the at least one nucleic acid encodes four exogenous induction factors. In one embodiment, the four encoded exogenous factors are Oct4, Sox2, Klf4, and c-Myc.

In some embodiments, the selection marker comprises the amino acid sequence of an enzyme or a fluorescent protein. In some embodiments, the enzyme comprises the amino acid sequence of a HSV thymidine kinase, a β-lactamase, or a luciferase.

In some embodiments, where the selection marker comprises the amino acid sequence of a β-lactamase, the above-mentioned method, further comprises contacting contacting the plurality of mammalian cells, after the culturing period, with a ratiometric fluorescent substrate that is cleaved by the β-lactamase.

In some embodiments, where the selection marker comprises the amino acid sequence of a fluorescent protein, the fluorescent protein comprises the amino acid sequence of a fluorescent timer or a photoactivatible fluorescent protein. In one embodiment, where the fluorescent protein comprises the amino acid sequence of a fluorescent timer, the method, further comprises detecting, at one or more time points, fluorescence in the plurality of mammalian cells at two emission wavelengths of the fluorescent timer.

In some embodiments, the selection marker comprises the amino acid sequence of an enzyme and a fluorescent protein. In some embodiments, where the selection marker comprises the amino acid sequence of an enzyme and a fluorescent protein, the enzyme comprises the amino acid sequence of a HSV thymidine kinase, and the fluorescent protein comprises the amino acid sequence of any of and (i) a fluorescent timer protein, (ii) a photoactivatible fluorescent protein, or (iii) EGFP.

In another aspect provided herein is a method for inducing differentiation of induced pluripotent stem cells, comprising depleting a population of induced pluripotent stem cells of cells that express at least one selection marker polypeptide, wherein expression of the selection marker polypeptide is driven by a promoter that undergoes transcriptional silencing in pluripotent stem cells, and differentiating the depleted population.

In some embodiments, the at least one selection marker polypeptide comprises two selection marker polypeptides.

In some embodiments, the selection marker polypeptide comprises the amino acid sequence of an enzyme. In some embodiments, the selection marker comprises the amino acid sequence of aHSV-thymidine kinase, a β-lactamase, or a luciferase. In some cases, where the selection marker polypeptide comprises the amino acid sequence of an enzyme, the selection marker polypeptide further comprises the amino acid sequence of a fluorescent protein.

In some embodiments, the selection marker polypeptide comprises the amino acid sequence of a fluorescent protein. In some embodiments, the fluorescent protein comprises the amino acid sequence of a fluorescent timer protein, a photoactivatible fluorescent protein, or EGFP.

In another aspect provided herein is a recombinant mammalian cell comprising one or more exogenous nucleic acids encoding:

(i) an induction factor; and

(ii) a selection marker polypeptide comprising the amino acid sequence of a β-lactamase, a fluorescent timer, a photoactivatible fluorescent protein, HSV-thymidine kinase, or a luciferase; wherein

expression of the encoded induction factor and the encoded selection marker is driven by a promoter comprising a retroviral silencing element, and wherein the exogenous nucleic acid encoding the selection marker is integrated in the genome of the recombinant mammalian cell.

In some embodiments, the recombinant mammalian cell is a recombinant human cell. In some embodiments, the human recombinant cell is a recombinant post-natal human cell.

In some embodiments, the promoter comprises the MoMLV LTR promoter.

In some embodiments, the one or more exogenous nucleic acids encode two or more of Oct4, Klf4, Sox2, c-Myc, Nanog, Lin-28, or functional homologs thereof. In some embodiments, the one or more exogenous nucleic acids encode three or more of Oct4, Klf4, Sox2, c-Myc, Nanog, Lin-28, or functional homologs thereof. In some embodiments, the one or more exogenous nucleic acids encode (i) Oct4, Klf4, Sox2, and c-Myc, or (ii) Oct4, Klf4, and Sox2, or functional homologs thereof.

In a further aspect provided herein is a nucleic acid comprising:
(i) a promoter comprising one or more retroviral silencing elements;
(ii) an open reading frame encoding an induction factor; and
(iii) an open reading frame encoding a fluorescent timer protein, a photoactivatable fluorescent protein, a β-lactamase, a luciferase, or a HSV-thymidine kinase, wherein (i) is operably connected to (ii) or (iii); and (ii) and (iii) are operably linked to each other.

In some embodiments, the promoter comprises a MoMLV LTR promoter.

In some embodiments, the encoded induction factor polypeptide comprises the amino acid sequence of an Oct4, Klf4, Sox2, c-Myc, Lin-28, or Nanog polypeptide.

In some embodiments, the above-mentioned open reading frame encoding an induction factor and the open reading frame encoding any of a fluorescent timer protein, a photoactivatable fluorescent protein, a β-lactamase, a luciferase, or a HSV-thymidine kinase are operably linked to each other by an IRES element or a nucleic acid sequence encoding a viral 2a peptide. In some embodiments, (iii) is an open reading frame encoding both a fluorescent timer protein and a HSV-thymidine kinase.

In some embodiments, the encoded fluorescent timer protein and HSV-thymidine kinase are operably connected to each other by an encoded viral 2a peptide.

In some embodiments, (iii) is an open reading frame encoding a fusion polypeptide comprising the amino acid sequence of (i) the HSV-thymidine kinase and the fluorescent timer protein; or (ii) the HSV-thymidine kinase and the β-lactamase.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an overview of an approach to the induction of iPS cells.

FIG. 2 is an overview of an exemplary, non-limiting, scheme for negative selection of iPS cells.

FIG. 3 (A) illustrates exemplary, non-limiting, silenceable expression vector (SEV) Expression Cassettes for Induction and Negative Selection; (B) illustrates exemplary SEV expression cassettes for dual selection marker expression.

FIG. 4 shows two exemplary retroviral packaging SEVs encoding Oct4-IRES-HSV-thymidine kinase and Sox2-IRES HSV-thymidine kinase useful for induction and negative selection of iPS cells.

FIG. 5 is an overview of an exemplary, non-limiting, scheme for combined iPS Cell Positive and Negative Selection.

FIG. 6 illustrates exemplary, non-limiting, SEV expression cassettes useful for combined positive and negative selection of iPS cells.

FIG. 7 shows exemplary retroviral packaging SEVs useful for combined positive and negative selection of iPS cells. The SEVs encode Oct4-IRES-Hygromycin phosphotransferase, Sox2-IRES puromycin N-acetyltransferase, Klf4-IRES-neomycin phosphotransferase, and c-Myc-IRES-thymidine kinase.

FIG. 8 (A) illustrates exemplary, non limiting, SEV “driver” and “selection” cassettes for negative selection based on indirect transcriptional silencing. The driver cassette encodes a ligand-regulated transactivator, the expression of which is driven by a promoter containing retroviral silencing elements (e.g., a MoMLV LTR promoter). The driver cassette once integrated into the genome is sensitive to epigenetic silencing in pluripotent stem cells. Expression from the selection cassette is dependent on continued expression of the ligand-dependent transactivator from the driver cassette. Epigenetic silencing of driver cassette expression therefore results in indirect silencing of selection cassette expression. 8(B) illustrates an exemplary, non-limiting, embodiment of negative selection, based on indirect silencing of selection marker expression over time in a fraction of a recombinant cell population.

### DETAILED DESCRIPTION OF THE INVENTION

### Overview

The present disclosure features methods and compositions for selecting pluripotent stem cells from a population of mammalian somatic cells undergoing a process to induce pluripotency (also commonly referred to as “nuclear reprogramming”). Pluripotent stem cells have the ability to differentiate into cells of all three germ layers (ectoderm, mesoderm and endoderm).

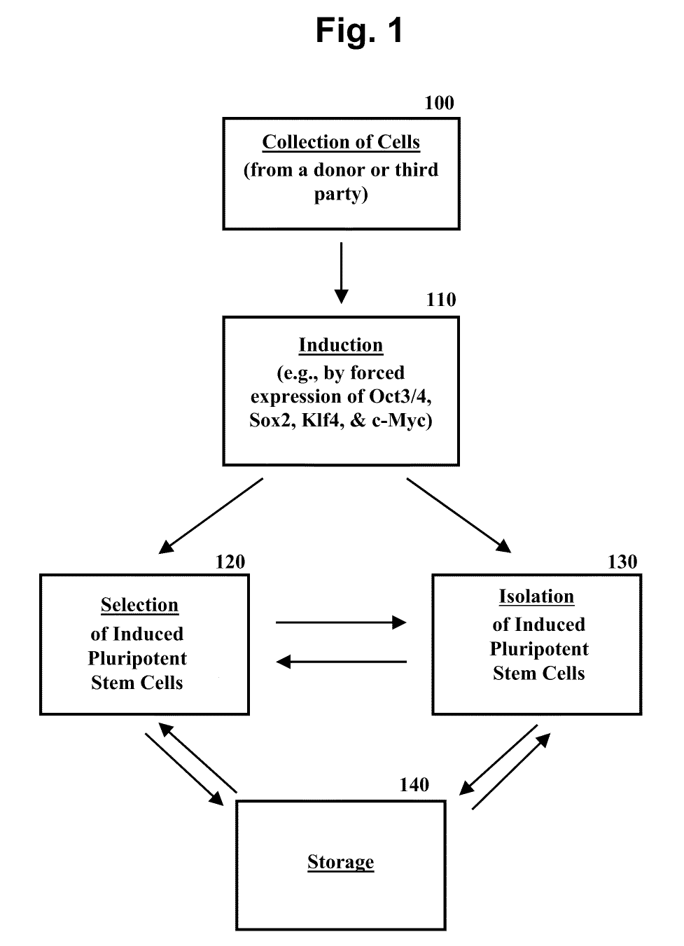

The process of inducing mammalian somatic cells to become pluripotent stem cells, referred to herein for convenience as “induced pluripotent stem cells” or iPS cells, is based on forcing the expression of exogenous or endogenous polypeptides, particularly proteins that play a role in maintaining or regulating self-renewal and/or pluripotency of ES cells. Examples of such proteins are the Oct4, Sox2, Klf4, and c-Myc transcription factors, all of which are highly expressed in ES cells. Forced expression may include in any combination introducing expression vectors encoding polypeptides of interest into cells, transduction of cells with recombinant viruses, introducing exogenous purified polypeptides of interest into cells, contacting cells with a non-naturally occurring induction agent that induces expression of an endogenous gene encoding a polypeptide of interest (e.g., Oct4, Sox2, Klf4, or c-Myc), contacting the cells with one or more inducing agents, induction factors, or any other biological, chemical, or physical means to induce expression of a gene encoding a polypeptide of interest (e.g., an endogenous gene Oct4, Sox2, Klf4, or c-Myc). For example, in some cases (depending on the cell type to be induced), one or more exogenous induction factors may be combined with chemical inducing agents such as a histone deacetylase inhibitor (e.g., valproic acid), a histone methyltransferase inhibitor (e.g., BIX01294), a DNA demethylating agent (e.g., 5-azacytidine), an L-type calcium channel agonist (e.g., BayK8644), kenpaullone (see Lyssiotis et al (2009), Proc. Nat. Acad Sci USA, (e-publication, 2009), or a TFG-β receptor inhibitor. Some basic steps to induce the cells are shown in FIG. 1. These steps may involve: collection of cells from a donor, e.g., a human donor, or a third party (100); induction of the cells, e.g., by forcing expression of polypeptides such as Oct4, Sox2, Klf4, and c-Myc (110); selecting pluripotent stem cells (120); isolating colonies (130); and optionally, storing the cells (140). Interspersed between all of these steps are steps to maintain the cells, including culturing or expanding the cells. In addition, storage of the cells can occur after many steps in the process. Cells may later be used in many contexts, such as therapeutics or other uses (150).

Embryonic stem (ES) cells are both self-renewing and pluripotent. iPS cells are also self-renewing and pluripotent. However, in contrast to ES cells, the induced cells can be derived from a wide range of cells and tissue, including non-embryonic tissue.

iPS cells have many uses. They may be subjected to conditions that enable them to generate differentiated cells, e.g., neurons, hepatocytes, or cardiomyocytes. They may also give rise to other types of stem cells, e.g., neural stem cells, hepatic stem cells, or cardiac stem cells, that have the ability differentiate into other cells of a specific lineage. The induced cells, and cells differentiated from them, are also useful for medical therapies such as cell replacement therapies. Since the induced cells can be induced from non-embryonic cells, a cell therapy can involve providing a subject with cells derived from his or her own tissue, thereby lessening the possibility of immune rejection.

This disclosure describes methods and compositions for selecting induced pluripotent stem cells. The disclosure further describes methods for maintaining the pluripotency of a population of induced pluripotent stem cells, and for maintaining the differentiated phenotype of a population of cells differentiated from induced pluripotent stem cells.

Selection of Induced Cells

In some cases a population of induced mammalian cells or mammalian cells to be induced may be genetically modified to facilitate identification or selection based on one or more selection criteria tested at one or more time points after the beginning of induction. Such selection criteria are useful for detecting iPS cells. Examples of such selection criteria include, but are not limited to, epigenetic changes or epigenetic states associated with pluripotency, the expression in the cells of one or more exogenous induction factors encoded by one or more expression vectors, expression of one or more selection markers, or expression of one or more stem cell marker genes or reporter genes indicating expression of a stem cell marker gene.

Cells having increased potency compared to differentiated cells, e.g., pluripotent cells (e.g., embryonic stem cells) exhibit specific epigenetic states that silence genomic gene expression driven by promoters comprising transcriptional elements present in the long terminal repeat (LTR) promoters of a number of retroviruses such as MoMLV and lentiviruses. See, e.g., Yao et al (2004), Mol Ther, 10(1):27-36. For convenience, such elements are referred to herein as retroviral silencing elements (RSEs) without, however, limiting such elements strictly to those that occur naturally in retroviruses or lentiviruses. In contrast to pluripotent cells, many differentiated cell types, e.g., cells to be induced as described herein, such as fibroblasts, are generally permissive for genomic gene expression driven by promoters comprising RSEs, e.g., MoMLV LTR promoter-driven gene expression. Thus, for a period after the beginning of induction, the induced cells described herein are permissive for expression of transgenes (e.g., selection markers or induction factors) the expression of which is driven by a promoter (e.g., the MoMLV 5′ LTR promoter, as shown below) comprising, inter alia, one or more RSEs (RSE promoter).

### MoMLV 5′ LTR Promoter Sequence

(SEQ ID NO: 1)
Cccgaaaagtgccacctgcataatgaaagaccccacctgtaggtttggc
aagctagcttaagtaacgccattttgcaaggcatggaaaaatacataact
gagaatagaaaagttcagatcaaggtcaggaacagatggaacagctga
atatgggccaaacaggatatctgtggtaagcagttcctgccccggctcag
ggccaagaacagatggaacagctgaatatgggccaaacaggatatctgtg
gtaagcagttcctgccccggctcagggccaagaacagatggtccccag
atgcggtccagccctcagcagtttctagagaaccatcagatgtttccag
ggtgccccaaggacctgaaatgaccctgtgccttatttgaactaaccaa
tcagttcgcttctcgcttctgttcgcgcgcttctgctccccgagctcaat
aaaagagcccacaacccctcactcggcgcgccagtcctccgattga
ctgagtcgcccgggtacccgtgtatccaataaaccctcttgcagttgcat
ccgacttgtggtctcgctgttccttgggagggtctcctctgagtgattga
ctacccgtcagcgggggtctttcatttgggggctcgtccgggatcggg
agacccctgcccagggaccaccgacccaccaccgggaggtaagct
ggccagcaacttatctgtgtctgtccgattgtctagtgtctatgactg
attttatgcgcctgcgtcggtactagttagctaactagctctgtatctgg

While not wishing to be bound by theory, it is believed that in induced cells that progress to an altered epigenetic state similar to that of pluripotent cells, RSE promoter-driven transgene expression will be transient since silencing occurs in induced cells that acquire increased potency (e.g., pluripotency) over a period of time following the beginning of induction. This transient time course of transgene expression permits initial expression of exogenous selection markers and, in some embodiments, exogenous induction factors, and is then followed by epigenetic silencing of transgene expression.

Accordingly, selection may utilize mammalian expression vectors (e.g., retroviruses or lentiviruses) comprising selection marker expression cassettes subject to transcriptional silencing. For convenience, such vectors are referred to herein as silenceable expression vectors (SEVs). In some cases, the SEV is subject to direct transcriptional silencing, which refers to transcriptional silencing mediated directly by an epigenetic state of the cell. In other cases, the SEV is subject to “indirect transcriptional silencing.” As used herein, indirect transcriptional silencing refers to a two-part expression control system, in which a first vector is an SEV comprising an inducible promoter, and a second SEV vector comprises a promoter that contains an RSE operably linked to an ORF for a transactivator capable of driving expression of the above-mentioned inducible expression cassette from the first SEV vector. Examples of suitable inducible promoters include, but are not limited to, a tetracycline-regulated promoter, an ecdysone-regulated promoter, or a mifepristone-regulated promoter. Thus, in this scheme, expression of the transactivator from the second, RSE-bearing, SEV is subject to direct silencing to changes in the epigenetic state of the cell. Subsequently, expression from the first SEV will be subject to indirect silencing, as expression from the first SEV is dependent on the continued expression of the cognate transactivator.

In some cases, an SEV or the selection marker expression cassette it contains is integrated into the genome of the host induced cell and are capable of undergoing direct epigenetic silencing in pluripotent cell types (e.g., iPS, ES, or embryonic carcinoma cells). In some cases the SEV is delivered as an integration-competent expression virus, e.g., a retrovirus or lentivirus. Suitable SEV viruses include those derived from MoMLV, HIV, SIV, FIV, AAV, or any other integration-competent expression virus, e.g., hybrid adenovirus-AAV viruses described in Goncalvnes et al (2008), PLoS ONE, 3(8):e3084. Alternatively, the SEV may a non-viral, transposon-based genomically integrating plasmid vector. See, e.g, Kaji et al, Mar. 1, 2009, Nature Epublication; Woltjen et al, Mar. 1, 2009, Nature Epublication; and Cadinanos et al (2007), Nuc. Acid Res., 35(12)e87.

Suitable silenceable promoters for SEV expression cassettes include those that contain at least one RSE, e.g., the MoMLV 5′ LTR sequence shown above. Examples of suitable RSEs include the negative control region (NCR), the direct repeat enhancer (DR), CpG Rich 5′ LTR Sequence, and the primer binding site (PBS). Their respective sequences are shown below:

### Negative Control Region (NCR) Sequence:

(SEQ ID NO: 2)

ccccacctgt aggtttggca agctagctta agtaacgcca
ttttgcaagg catgga

### Direct Repeat Enhancer (DR) Sequence:

(SEQ ID NO: 3)

aaggtcagga acagatggaa cagctgaata tgggccaaac
aggatatctg tggtaagcag ttcctgcccc ggctcagggc
caagaacaga tg

### Primer Binding Site:

(SEQ ID NO: 4)

cgggggtctt tcatttgggg gctcgtccgg gat

### CpG-Rich 5′ LTR Sequence:

(SEQ ID NO: 5)
gttc gcttctcgct tctgttcgcg cgcttctgct ccccgagctc
aataaaagag cccacaaccc ctcactcggc gcgccagtcc
tccgattgac tgagtcgccc gggtacccgt gtatccaata
aaccctcttg cagttgcatc cgacttgtgg tctcgctgtt
ccttgggagg gtctcctctg agtgattgac taccc

Negative Selection of Induced Cells

In some embodiments, after an induction period sufficient to allow epigenetic silencing of SEV-mediated selection marker expression, induced cells and colonies are selected based on the absence of expression or very low expression of one or more selection markers (e.g., Herpes Simplex Virus Thymidine Kinase (HSV-thymidine kinase) or a multifunctional reporter protein such as TK-TIMER), which is referred to herein as “negative selection.” While not wishing to be bound by theory, it is believed that negative selection, as described herein, can be applied to cells induced by any induction method insofar as the induction method initiates progressive epigenetic silencing of selection marker expression in at least a fraction of induced cells. Examples of induction methods include, but are not limited to, viral transduction of induction factor transgenes as described herein, DNA transfection of induction factor transgenes (e.g., transposon-mediated transgenesis), protein transduction of induction factor polypeptides (see, e.g., Kim et al (2009), Cell Stem Cell, e-publication) incubation with inducing agents (e.g., TGF-β receptor inhibitors, BIX-01294 and BayK8644; kenpaullone, or RNAi or RNAa), or any combination thereof. See, e.g., Maherali et al (2008), Cell Stem Cell, 3(6):595-605.

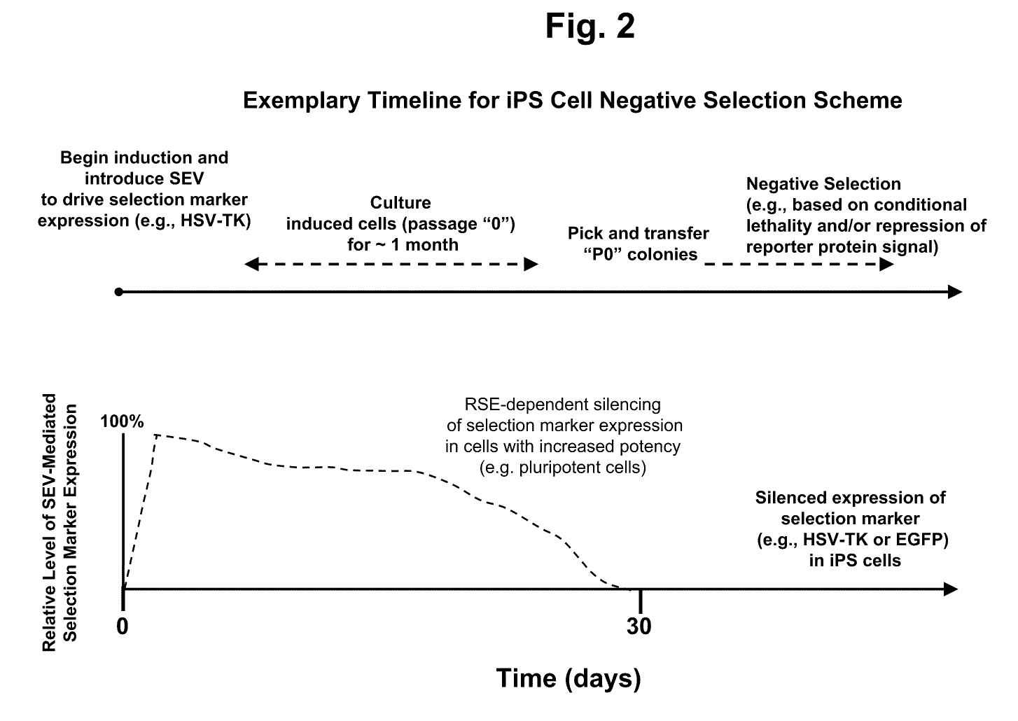

A schematic overview of negative selection of iPS cells is provided in FIG. 2. SEV-mediated selection marker expression or activity is evaluated in induced cells at least one time points following the beginning of the induction period.

In some cases, induced pluripotent stem cells are identified by a method that includes introducing into the genome of a plurality of mammalian cells (e.g., human post-natal cells) undergoing induction of pluripotency an expression vector (e.g., an SEV) encoding a selection marker operably linked to a promoter that drives expression of the encoded selection marker in the plurality of mammalian cells and is capable of undergoing transcriptional silencing in a pluripotent stem cell. The plurality of mammalian cells in which the above-mentioned expression vector is to be introduced: contain at least one exogenous nucleic acid encoding at least one induction factor; (ii) at least one exogenous induction factor (e.g., a purified polypeptide introduced directly into the cells); (iii) are contacted with at least one inducing agent; or (iv) meet any combination of conditions (i)-(iii). Afterwards, activity or expression of the encoded selection marker is assayed in the cultured cells. Where activity or expression of the selection marker is low or absent in one more of the cultured cells, those cells are considered to be candidate iPS cells.

Depending on the selection marker used for negative selection, selection marker expression or activity are assayed at a time point at least about two days to about two months, e.g., about 3 days, 4 days, 5 days, 7 days, 8 days, 10 days, 12 days, 16 days, 18 days, 20 days, three weeks, 24 days, 25 days, 28 days, one month, 35 days, 40 days, 50 days, or another time point from at least about two days to about two months following the beginning of induction. In some embodiments, where selection marker activity can be assayed non-invasively (e.g. by fluorescence microscopy in the case of a fluorescent protein), selection marker activity levels are determined at least two time points following the beginning of induction. Non-invasive selection marker assays may be conducted at various time intervals following the beginning of induction to a final round of selection. Assay intervals may be from about every 8 hours to about every 14 days, e.g., about every 2, 3, 4, 7, 10, 12, 14 days, or another time interval from about 8 hours to about 14 days. At early time points during the induction period, selection marker assays are useful to identify induced cells in which a selection marker has been successfully introduced. At later time points, selection marker assays are carried out to identify and select induced cells in which selection marker expression is progressively silenced. Regular monitoring of selection marker activity allows identification of induced cells undergoing epigenetic silencing as early on as possible. In some embodiments, non-invasive monitoring of selection marker expression is used to determine induced cell response to agents that may accelerate or increase the efficiency of the transition to pluripotency (“induction-agents,” e.g., methyltransferase inhibitors, and RNAi against lineage-specific transcription factors) at time points greater than six days past the beginning of induction. In some embodiments, non-invasive monitoring of selection marker expression over time is used to identify novel induction-facilitation agents, which are identified based on an increase in the fraction of induced cells that exhibit silencing of selection marker expression, and/or the rate at which selection marker expression is silenced.

During at least one time point after the beginning of induction, the induced cells undergo negative selection based on low or absent expression of a selection marker to identify induced cells that have acquired pluripotency, and may then be further selected, expanded, stored, or characterized further as described herein. In some cases, cells undergo selection at about 14 days to about 50 days following the beginning of induction, e.g., about 22 days to 40 days, 25 days to 35 days, 27 days to 32 days, or another time point from about 14 days to about 50 days following the beginning of induction. Depending on the selection method used, multiple rounds of selection may be performed. For example, where selection is based on a non-invasive selection marker assay, e.g., FACS sorting of fluorescent protein-positive and fluorescent protein-negative induced cells, negative selection of induced cells by the non-invasive selection method is performed multiple times to obtain a subpopulation of induced cells that have low or absent selection marker expression.

Where selection is based on conditional lethality of a selection marker activity (e.g., thymidine kinase activation of the cytotoxin prodrug ganciclovir), induced cells are incubated in the presence of a prodrug for at least about 16 hours to at least about 7 days, e.g., at least about 1, 2, 3.5, 4, 5, days, or another period from at least about 16 hours to at least about 7 days. Afterwards, surviving colonies or cells may be expanded, further characterized, or stored as described herein.

Thus, in some cases, a method for selecting an induced pluripotent stem cell from a plurality of recombinant mammalian cells after induction, includes culturing the plurality of recombinant mammalian cells for a period following the induction, and contacting the plurality of recombinant mammalian cells with a cytotoxin prodrug, where the plurality of recombinant mammalian cells comprise within their genome an exogenous nucleic acid that includes a promoter containing a retroviral silencing element operably linked to an open reading frame for an enzyme that converts the cytotoxin prodrug into a cytotoxin. A mammalian cell that survives contact with the cytotoxin prodrug is then considered to be a candidate iPS cell.

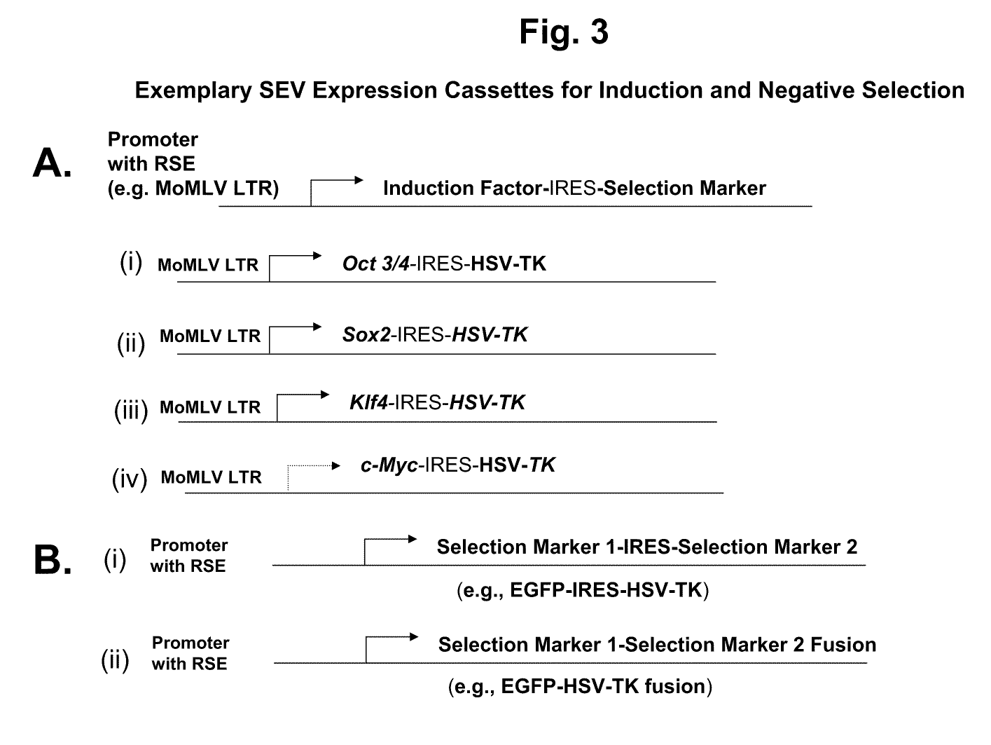

In some cases, negative selection includes introducing into the cells being induced an SEV comprising an induction factor (e.g., Oct4) open reading frame (ORF) operably linked by an internal ribosomal entry site (IRES) or viral 2a peptide-encoding sequence to a selection marker ORF, as schematized in FIG. 3(A) for an exemplary embodiment. Thus, negative selection may, in some cases, be integrated with induction of pluripotency. Accordingly, in some embodiments, a method for generating an induced pluripotent stem cell includes introducing into a plurality of mammalian cells at least one integration-competent expression vector that contains a selection marker open reading frame operably linked to a promoter capable of undergoing transcriptional silencing in a pluripotent stem cell, and:
(i) introducing into the plurality of mammalian cells an expression vector for one or more induction factors;
(ii) introducing into the plurality of mammalian cells one or more induction factor polypeptides;
(iii) contacting the plurality of mammalian cells with one or more inducing agents; or
(iv) any combination of (i)-(iii).

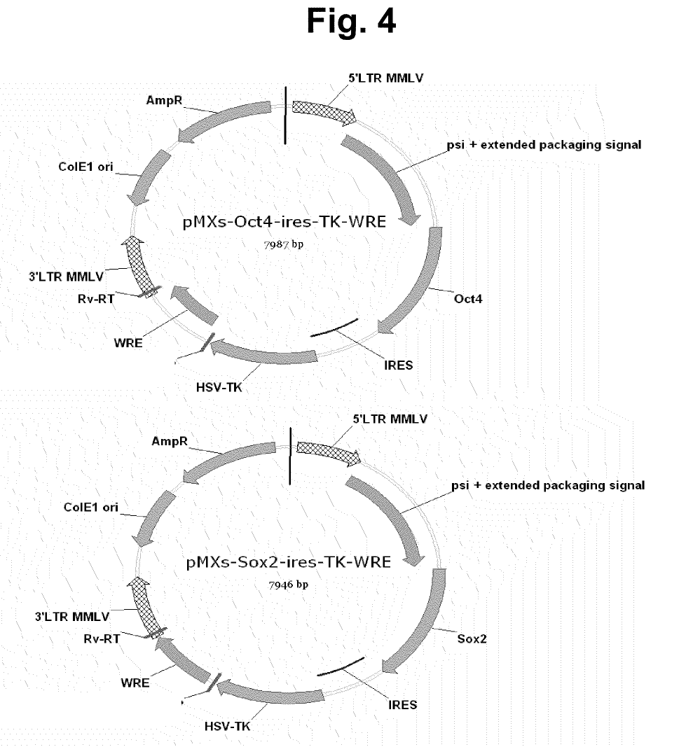

In other cases, an SEV comprises ORFs for one or more selection markers, but no induction factor ORF, as depicted schematically for some exemplary embodiments in FIG. 3(B). Exemplary embodiments of viral packaging plasmids used for construction of SEV retroviruses used for negative selection of iPS cells are illustrated in FIG. 4. Their nucleotide sequences are provided in appendix included in the Examples section.

In some cases, the selection marker comprises an amino acid sequence at least 70% to 100%, e.g., 80%, 85%, 90%, 95%, or another percent from about 70% to 100% identical to the amino acid sequence of an enzyme, a fluorescent protein, or a cell surface protein tag selection markers. Selection markers for negative selection may be a selection marker polypeptide that comprises the amino acid sequence of an enzyme (selection marker enzyme). In some embodiments, the selection marker enzyme is a conditionally lethal enzyme (CLE) that kills cells only in the presence of a prodrug substrate, which is then converted into a cytotoxin by the CLE. Thus, only induced cells and colonies that lack expression of the CLE (e.g., pluripotent cells that silence CLE expression) can survive in the presence of the prodrug substrate for the CLE. Examples of suitable CLEs and their prodrug substrates include, but are not limited to, a herpes simplex virus thymidine kinase (HSV-thymidine kinase) and its prodrug ganciclovir, e.g., a truncated HSV-TK, the sequence of which is shown below:

(SEQ ID NO: 6)
MASYPCHQHASAFDQAARSRGHNNRRTALRPRRQQKATEVRLEQKMPT
LLRVYIDGPHGMGKTTTTQLLVALGSRDDIVYVPEPMTYWRVLGASETIA
NIYTTQHRLDQGEISAGDAAVVMTSAQITMGMPYAVTDAVLAPHIGGEAG
SSHAPPPALTLIFDRHPIAALLCYPAARYLMGSMTPQAVLAFVALIPPTL
PGTNIVLGALPEDRHIDRLAKRQRPGERLDLAMLAASPRLWAACQYG
AVSAGRRVVAGGLGTAFGGGRAAPGCRAPEQRGPTTPYRGHVIYPVS
GPRVAGPQRRPV

Other CLE/prodrug combinations include cytosine deaminase (GenBank No. AAB67713) (prodrug: 5-fluorocytosine); carboxypeptidase G2 (prodrug: 4-[(2-Mesyloxyethyl)-(2-chloroethyl)amino benzoic acid); and penicillin amidase (prodrug: N-(4′-Hydroxyphenylacetyl)palytoxin. In some embodiments, where the CLE comprises the amino acid sequence of HSV-thymidine kinase, selection can be carried out in the presence of ganciclovir at a concentration of about 0.5 μM to about 10 μM, e.g., about 1, 1.5, 2, 2.5, 3, 4, 5, 7, 8, or another concentration from about 0.5 to 10 μM ganciclovir.

In other cases, a selection marker enzyme converts a substrate that, in the process, yields a detectable signal, e.g., a fluorescent or luminescent signal in the presence of a fluorogenic or luminogenic substrate, respectively. For convenience, such selection marker enzymes are termed “reporter enzymes” herein. For example, in some embodiments, the selection marker enzyme comprises the amino acid sequence of a luciferase, e.g., a firefly luciferase, click beetle luciferase, or Renilla luciferase. Luciferase activity can be detected by providing an appropriate luminogenic substrate, e.g., firefly luciferin for firefly luciferase or coelenterazine for Renilla luciferase. Luciferase activity in the presence of an appropriate substrate can be quantified by luminometry to assay total luciferase activity of whole cell populations in culture dish wells, or, alternatively, luciferase activity of individual cells or colonies can be detected by use of a microscope in combination with a photon counting camera. Details of luciferase assays, including high-throughput methods, are disclosed in, e.g., U.S. Pat. Nos. 5,650,135, 5,744,320, and 6,982,431. In other embodiments, the selection marker enzyme comprises the amino acid sequence of a modified β-lactamase, the expression of which can be detected and quantified in living cells by a ratiometric fluorescence assay for breakdown of fluorogenic β-lactam substrates as described in, e.g., U.S. Pat. Nos. 5,741,657, 6,031,094, and U.S. Patent Publication No. 20070184513. See also Qureshi (2007), Biotechniques, 42(1):91-96 for a review. In brief, the fluorogenic β-lactam substrate is a ratiometric fluorescent substrate the emission wavelength of which is shifted from green to blue emission upon cleavage by β-lactamase. As the assay is ratiometric, it is insensitive to variations in substrate loading, which may otherwise perturb measurement of the β-lactamase reporter assay. Other suitable reporter enzymes include, but are not limited to, the Halo-Tag® hydrolase (Promega, Madison, Wis., as described in, e.g., U.S. Pat. No. 7,238,842 and Patent Publication Nos. 20080026407 and 20080145882) and β-galactosidase.

In some cases, the selection marker polypeptide may comprise the amino acid sequence of an exogenous cell surface protein not normally expressed on the surface of the cells to be induced or that of pluripotent stem cells. Such markers include, e.g., a truncated CD4 surface marker as described in, e.g., Gaines et al (1999), Biotechniques, 26(4):683-688. During negative selection, induced cells expressing such markers on their cell surface can then be sorted from non-expressing (e.g., silenced) induced cells by Magnetism Activated Cell Sorting (MACS) as described in, e.g., U.S. Pat. No. 5,691,208.

A selection marker polypeptide may comprise the amino acid sequence of a fluorescent protein. Examples of suitable fluorescent reporter proteins include, but are not limited to, EGFP and its variants such as YFP, Cyan, and dEGFPs; DS-Red, monomeric Orange and its variants, including fluorescent timer as described in U.S. Pat. No. 7,217,789. Fluorescent timer proteins exhibit two time-dependent emission wavelengths: newly translated fluorescent timer proteins give fluorescence emission at a first wavelength (e.g., green), but as they mature over time emit fluorescence at a distinct wavelength (e.g., red). Thus, fluorescent timer proteins, provide a normalized readout of transcriptional activity, since as transcription and translation are reduced over time, as occurs in transcriptional silencing, the ratio of emission from newly translated/immature fluorescent timer (e.g. green emission) to emission from mature fluorescent timer (e.g., red emission) will decrease thereby providing a useful time-dependent index of conversion of the expressing cell to pluripotency that, unlike non-ratiometric reporter proteins, is independent of absolute levels of reporter protein expression. Eventually, upon complete transcriptional silencing both immature and mature forms of the fluorescent timer protein will be absent from the cell, and no fluorescence will be observable at either immature or mature emission wavelengths. In other embodiments, the fluorescent reporter protein comprises the amino acid sequence of a photoactivatible fluorescent protein, e.g., “kindling red fluorescent protein,” as described in Chudakov et al (2003), Nature Biotechnol, 21(2):191-194 and in U.S. Patent Application Publication No. 20030092884. Photoactivatible proteins, and particularly reversible photoactivatible proteins are useful in the methods described herein, as their fluorescence will not interfere with downstream reporter proteins that may subsequently engineered into induced pluripotent stem cell lines. Other suitable fluorescent proteins are known in the art as described in, e.g., Reporter Genes: A Practical Approach, ed. by Donald Anson, Humana Press (2007).

A selection marker may be a multifunctional polypeptide that comprises the amino acid sequences of more that one selection marker polypeptide. Examples of suitable multifunctional selection marker polypeptides include, but are not limited to, fusion polypeptides comprising the amino acid sequence of an enzyme and a fluorescent protein. Such fusion polypeptides are advantageous in that their expression is easily detected and followed over time during induction and permit selection of induced cells having increased potency based on both fluorescence assays and conditional lethality in the presence of a selection agent (e.g., ganciclovir). A multifunctional selection marker polypeptide may possess an activity or property that can be detected in live cells (e.g., fluorescence) and an activity that is conditionally lethal to cells (e.g., thymidine kinase conversion of the prodrug ganciclovir to the cytotoxin ganciclovir monophosphate). In some embodiments, the selection marker comprises the amino acid sequence of a CLE fused to the amino acid sequence of a fluorescent protein, e.g., any of the above-mentioned CLEs fused to any of the above-mentioned fluorescent protein selection marker polypeptides. In some embodiments, the selection marker comprises the amino acid sequence of HSV-thymidine kinase fused to the amino acid sequence of EGFP as described in, e.g., Ponomarev et al (2003), Neoplasia, 5(3):245-254. In other cases, the selection marker comprises the amino acid sequence of HSV-thymidine kinase fused to the amino acid sequence of fluorescent timer. In some cases, the selection marker comprises the amino acid sequence of a CLE fused to the amino acid sequence of an enzyme suitable for positive selection (e.g., hygromycin phosphotransferase, puromycin N-acetyltransferase, and neomycin phosphotransferase. Examples of dual enzyme selection marker polypeptides include, but are not limited to, those comprising the amino acid sequences of the following enzyme pairs: hygromycin phosphotransferase-thymidine kinase (see, e.g., Lupton et al (1991), Mol Cell Biol. 1991; 11(6):3374-3378); puromycin N-acetyltransferase-HSV-thymidine kinase (see, e.g., Chen et al (2000), Genesis, 28(1):31-35); and HSV-thymidine kinase-neomycin phosphotransferase (see, e.g., Candotti et al (2000), Cancer Gene Ther, 7(4):574-580. In some embodiments, where a selection marker contains the amino acid sequences of two selection marker polypeptides (e.g., HSV-thymidine kinase and EGFP), the amino acid sequences may be linked to each other by an intervening 2A peptide sequence of the foot-and-mouth disease virus (F2A) or 2A-like sequences from other viruses. See, e.g., Hasegawa et al (2007), Stem Cells, 25:1707-1712 and Symczak et al (2004), Nat Biotechnol, 589-594. Inclusion of the 2A peptide sequence allows post-translational cleavage of the polypeptide into separate selection marker polypeptides.

Live cell selection marker assays of induced cells can be conducted in any vessel suitable for mammalian cell culture (e.g., microtiter cell culture plates, multiwell plates, cell culture dishes, and cell culture flasks). Multi-well cell culture plates can be adapted for direct luminometry or fluorimetry of cells or in the wells of the plate. Luciferase activity can be measured in live cells by adding a suitable luciferase substrate directly to the cultured cells in cell culture medium (i.e., without a lysis step) and measuring light emission directly from the intact cells. Viviren™ substrate (Promega, WI) or other suitable cell-permeable luciferase substrates can be added directly to cells to measure luciferase activity.

Fluorescent polypeptides (e.g., EGFP) can be detected and quantified in live cells by a number of detection methods known in the art (e.g., fluorimetry or fluorescence microscopy). Details of reporter assays screens in live cells using fluorescent polypeptides, including high-throughput methods, can be found, e.g., in U.S. Pat. No. 6,875,578. In some cases, FACS analysis is particularly useful for rapid negative selection of induced cells where a selection marker is autofluorescent or has an activity that can yield a fluorescent signal (e.g., β-lactamase breakdown of CCF-2). Typically, in flow cytometry, cells (or cellular fragments) labeled with an internalized fluorescent moiety are passed through a slender flow cell along with a sheath fluid so that the cells flow in single file. The individual cells in the flow are irradiated one at a time with a light beam (such as a laser beam) by means of hydrodynamic focusing, and the intensity of scattered light or fluorescent light from the cells, e.g., light information indicative of the cells, is measured instantaneously to analyze the cells. Flow cytometry of this kind is advantageous in that a large number of cells can be analyzed at high speed and with great accuracy.

Flow cytometers are well known in the art and are commercially available from, e.g., Beckman Coulter and Becton, Dickinson and Company. Typical flow cytometers include a light source, collection optics, electronics and a computer to translate signals to data. In many cytometers, the light source of choice is a laser which emits coherent light at a specified wavelength. Scattered and emitted fluorescent light is collected by two lenses (one set in front of the light source and one set at right angles) and by a series of optics, beam splitters and filters, specific bands of fluorescence can be measured.

One known example of a cell analyzing apparatus using flow cytometry comprises a flow cell for forming a slender stream of liquid, a light source (such as a laser) for irradiating the cells which flow through the interior of the flow cell with a light beam, a photodetector for detecting cell light information from the cells irradiated with the light beam and converting the light information into an electric signal, a signal processing circuit for amplifying, integrating and removing noise from the signal produced by the photodetector, and a computer for processing a signal, which represents the cell light information, outputted by the signal processing circuit.

Skilled practitioners will appreciate that many variations and/or additions to basic flow cytometry systems can be made, e.g., providing practitioners with additional and/or different analyzing capabilities. Further, skilled practitioners will appreciate that flow cytometry can be performed in an automated manner and that a flow cytometer can be provided as part of a larger, automated system, e.g., a high-throughput system. The methods of the present invention contemplate the use of such apparatus and systems. Also included within the present invention is the use of any apparatus not known as a flow cytometer, but which performs essentially the same function as a flow cytometer (e.g., an imaging cytometer), as described above. FACS of a population of cells showing little or no expression of the selection marker used for negative selection can be repeated multiple times until an acceptably low fraction of the cells exhibits selection marker expression.

Positive Plus Negative Selection of Induced Cells

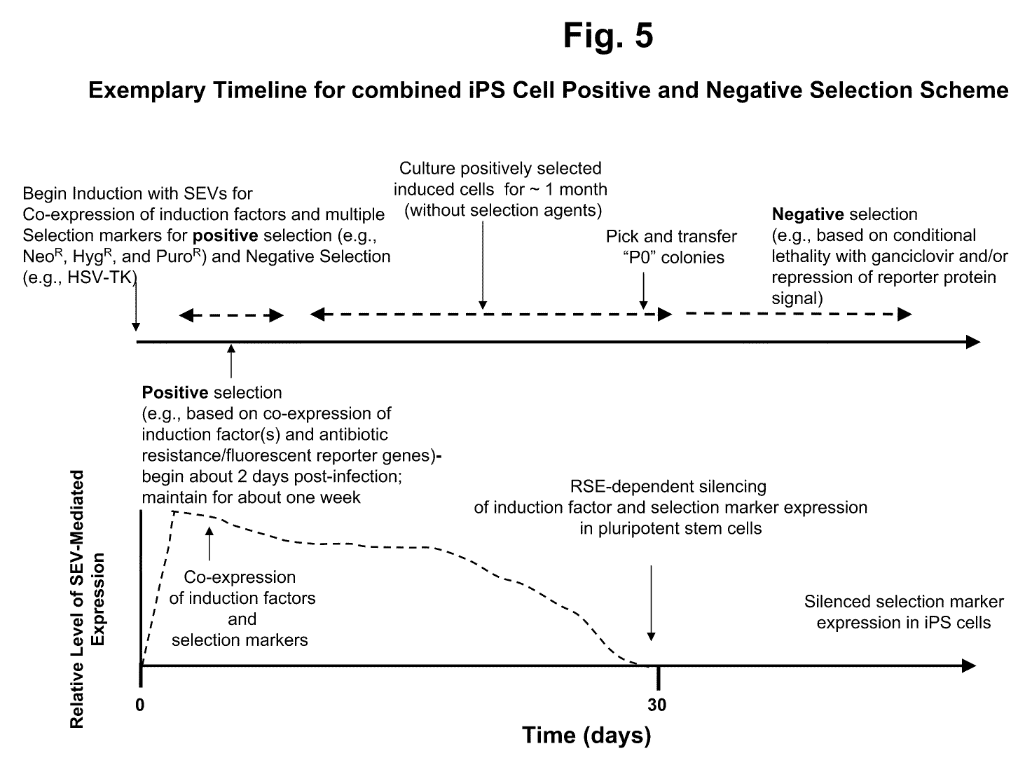

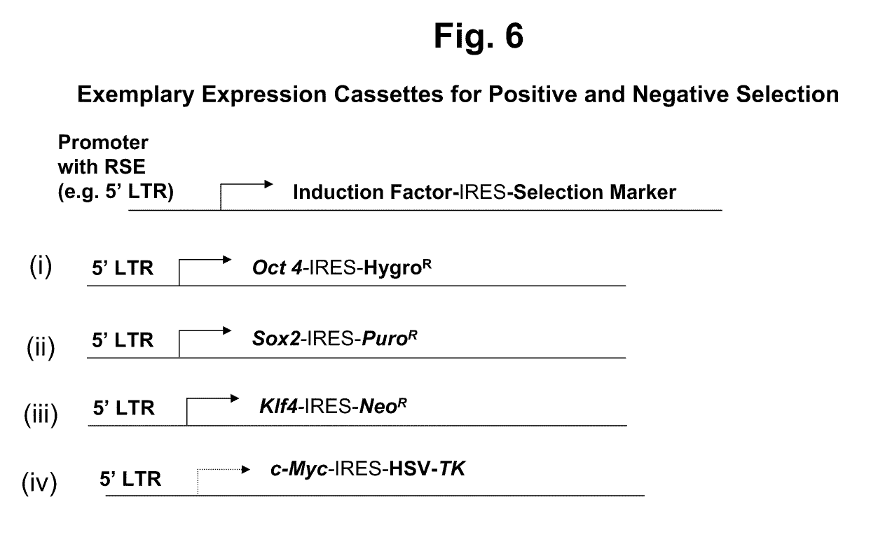

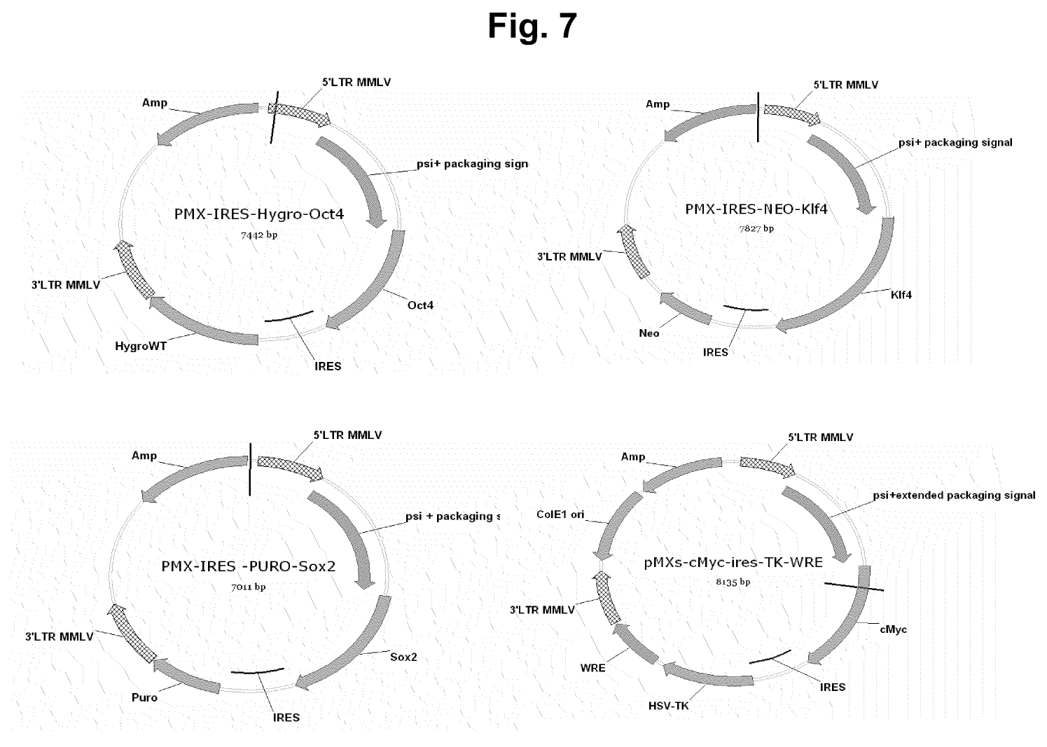

In some embodiments, negative selection of induced cells, as described above, is combined with positive selection of induced cells, as follows. Where exogenous induction factors (e.g., Oct4) SEVs are introduced as described herein to initiate induction of pluripotency, a distinct selection marker is co-expressed with each exogenous induction factor to allow for selection of cells that express all of the provided exogenous induction factors (“positive selection”). Early positive selection combined with subsequent negative selection of induced cells based on selection markers, is expected to increase the efficiency of obtaining putative pluripotent colonies or cells compared to selection of induced cells based on morphology alone. An overview of the combination of positive and negative selection of induced cells is depicted schematically in FIG. 5. Exemplary, non-limiting embodiments of expression cassettes, and viral vector constructs useful for this method are illustrated in FIGS. 6 and 7. After the beginning of induction, positive selection may begin from about 12 hours to about 36 hours following the beginning of induction, e.g., about 13, 14, 16, 18, 20, 24, 30, 32, or at another time point from about 12 to about 36 hours following the beginning of induction. Where the one or more selection markers confer resistance to one or more selection agents (e.g., an antibiotic such as hygromycin), the period of positive selection may last for at least about 3 to about 14 days, e.g., about 4, 5, 6, 7, 8, 9, 10, 12, 13, or another period from about 3 to about 14 days. Where multiple selection agents are to be used, e.g., neomycin, hygromycin, and puromycin, the selection agents may be used in combination during the selection period. In other embodiments, multiple selection agents may be used consecutively. Where one or more selection agents are used for positive selection, the selected induced cells are cultured in the absence of the selection agents following the above-mentioned positive selection period. Following positive selection, the selected colonies and cells are cultured as described herein until the beginning of a negative selection period as described above.

In some cases, the co-expression of a selection marker and an induction factor is accomplished by providing an SEV comprising sequences encoding both an induction factor and a selection marker, where the selection marker and induction factor sequences are operably linked to one another by an internal ribosome entry sequence (IRES) element or a viral 2a peptide-encoding sequence so as to yield a bicistronic mRNA encoding both the induction factor and a selection marker when expressed. In some embodiments, the encoded selection marker is in a position upstream from the IRES and the encoded selection marker is located downstream from the IRES. In other embodiments, the position of the selection marker is upstream of the IRES and the encoded induction factor is located after the IRES. Some exemplary embodiments, of SEVs useful for combined positive and negative selection of iPS cells are provided in FIGS. 6 and 7.

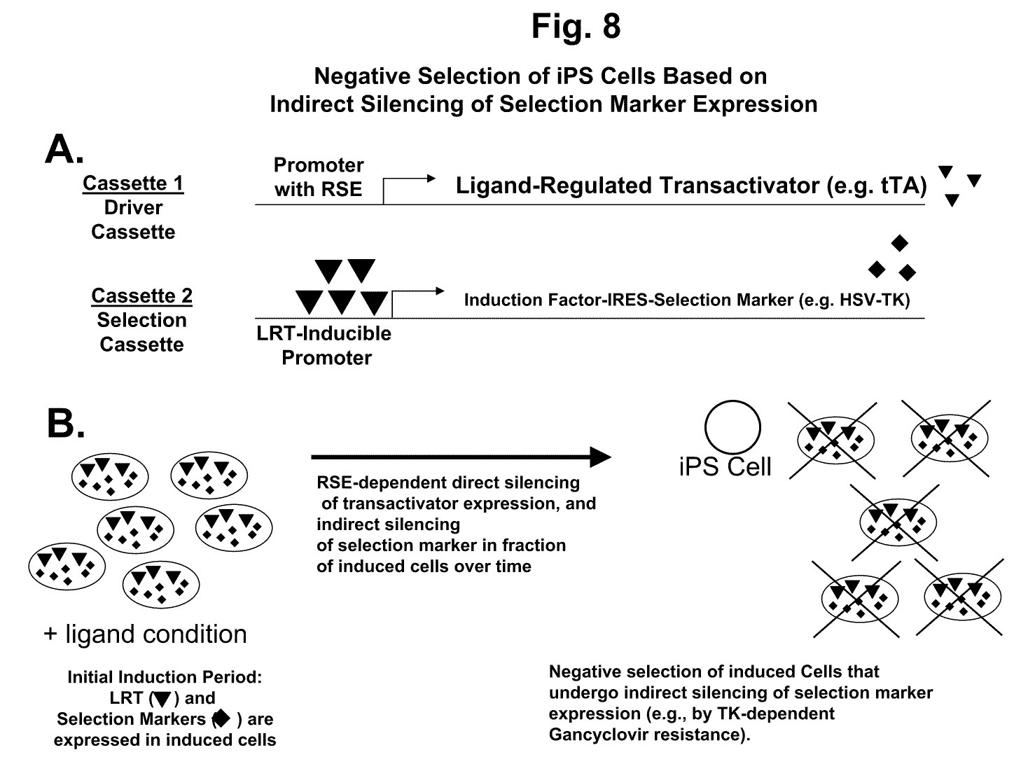

In some cases, the co-expression of an exogenous induction factor linked by an IRES element to a selection marker is driven by a promoter that is inducible by a ligand-regulated transactivator (LRtA). Examples of suitable LRTs include, but are not limited to, tet transactivator (tTA), reverse tTA (rtTA), or RU486-inducible transactivator (Tsai et al (1998), Adv Drug Deliv Rev, 30(1-3):23-31). Where, expression of an induction factor and/or selection marker is inducible by an LRT, the LRT is provided on an SEV, so that expression or lack thereof of the LRtA will directly depend on the epigenetic state (i.e., silencing capability) of the host cell, which will, in turn, modulate the expression of LRtA-inducible gene expression. Thus, where an LRtA is used to drive expression of selection markers or induction factors, silencing of induction factor or selection marker expression will be indirect silencing that results form direct silencing of LRtA expression. This is illustrated schematically in FIG. 8.

Suitable selection markers for positive selection include, but are not limited to, any polypeptide comprising an amino acid sequence at least 70% to 100%, e.g., 80%, 85%, 90%, 95%, or another percent from about 70% to 100% identical to the amino acid sequence of the selection marker polypeptides described in the previous section.

In some embodiments, induction is initiated by introducing into a population of mammalian cells a first SEV for expression of a selection marker suitable for negative selection as described above (e.g., HSV-thymidine kinase), and second, third, and fourth SEVs comprising ORFs for the induction factor polypeptides, Oct4, Klf4, and Sox2, as described herein, where each induction factor ORF is operatively linked via an IRES sequence to the ORF for a different selection marker suitable for positive selection, as described herein. In some embodiments, the selection markers to be co-expressed with the Oct4, Klf4, and Sox2 induction factors are, hygromycin phosphotransferase, neomycin phosphotransferase, and puromycin N-acetyltransferase. In some cases, an additional SEV is transduced into the cells that encode an additional induction factor polypeptide, c-Myc, operatively linked to a selection marker.

Selection of iPS Cells without Selection Markers

Pluripotent stem cells are characterized by a significantly shorter G1 cell cycle (2.5-3 hours) relative to that of somatic cells. See, e.g., Becker et al (2006), J Cell Physiol, 209(3):883-893. Thus, the abbreviated G1 kinetics of iPS cells may be used as a selection criterion to isolate pluripotent cells from a background of somatic cells, thereby avoiding the need to introduce an expression vector encoding a selection marker. Accordingly, in some cases, a population of cells that has undergone induction is cultured for a period from at least about 10 days to about 50 days, e.g., about 14 days to about 40 days, about 21 days to about 30 days, or another period from at least about 10 days to about 50 days. At the end of this culturing period, the cultured induced cells are cell-cycle synchronized by arrest in the G2/M transition with an appropriate cell cycle inhibitor. Examples of such cell cycle inhibitors include, but are not limited to, nocodazole (about 100 ng/ml) or demeocolcine (about 20 ng/ml). The cultured cells are contacted with the cell cycle inhibitor for a period of about 10 to about 16 hours (e.g., about 11, 12, 13, 14, or another period from about 10 to about 16 hours). Subsequently, the cell cycle inhibitor is washed out and the cells are cultured for a period of about 3 to about 8 hours (e.g., about 3.5, 4, 4.5, 5, 6, 6.5, or another period from about 4 to about 8 hours in growth medium and then incubated in the presence of a cell-permeable fluorescent nucleic acid stain for about 30 to 90 minutes. In some embodiments, the nucleic acid stain is Hoechst 33342 (at a concentration of about 5 μg/ml). Staining, may, in some cases, include adding verapamil (50 μM) to prevent cell efflux of the DNA stain. The cells are washed in cell culture medium or a physiological buffer and then sorted by FACS based on DNA content. Methods for FACS based on DNA content are known in the art. See, e.g., Darzynkiewicz et al (2001), Curr Protoc Cell Biol.: Chapter 8:Unit 8.4. A cell population with 2N content is enriched for iPS cells due to their shorter G1 phase and earlier entry into S phase. In some cases, FACs sorting or the entire procedure may be repeated for further enrichment of iPS cells. In some cases, the selection method is performed on induced cells that have undergone induction without the use of exogenous c-Myc. In other cases, the selection is performed on induced cells that have undergone forced expression of c-Myc (e.g., by introduction of an exogenous nucleic acid for expression of c-Myc).

Methods for Identifying Inducing Agents

In some cases, methods and compositions suitable for selection of iPS cells are used to screen for agents or combinations of agents for inducing a pluripotent stem cell. Such a screening method may include the steps of: (i) providing a plurality of recombinant mammalian cells comprising a genomically integrated exogenous nucleic acid containing an open reading frame that encodes a selection marker and is operably linked to a promoter comprising one or more retroviral silencing elements, where the selection marker is expressed in the plurality of recombinant mammalian cells; (ii) culturing the plurality of recombinant mammalian cells in the presence of one or more test agents; and (iii) determining an expression level or activity of the selection marker in the plurality of recombinant mammalian cells after the culturing step. A progressive reduction of expression level or activity of the selection marker expression in at least one of the plurality of cultured cells in the presence of the one or more test agents compared to the selection marker expression level or activity in the absence of the one or more test agents indicates that one or more test agents are inducing agents. In some cases, the promoter comprises a MoMLV LTR promoter. Suitable encoded selection markers include, but are not limited to enzymes (e.g., of HSV thymidine kinase, cytosine deaminase, carboxypeptidase G2, β-lactamase, alkaline phosphatase, β-galactosidase, or a luciferase) and fluorescent proteins (e.g., EGFP, fluorescent timer, a monomeric DS-Red, monomeric Orange, YFP, or Cyan). Where the selection marker comprises the amino acid sequence of HSV-thymidine kinase, the method may also include selection of cells in the presence of the cytotoxin prodrug ganciclovir.

In some cases, the recombinant mammalian cells used in the screening method, may also include one or more exogenous nucleic acids encoding at least one, but no more than three induction factors. Such encoded induction factors may include, e.g., Oct4, Sox2, Klf4, Lin-28, or Nanog polypeptides.

In some embodiments, the agent to be tested is an siRNA, including, but not limited to, a double stranded RNA that comprises about 19 base pairs of a target gene sequence and is capable of inhibiting target gene expression of RNA interference. See, e.g., Scherr et al., (2007), Cell Cycle, 6(4):444-449. In some embodiments, the siRNAs to be assayed include, but are not limited to, whole-genome siRNA libraries, as described in, e.g., Miyagishi et al., (2003), Oligonucleotides, 13(5):325-333; and Huesken et al., (2005), Nat. Biotechnol., 8:995-1001. Suitable whole genome siRNA libraries, e.g., arrayed siRNA libraries that are commercially available include, the “Human Whole Genome siRNA Set V4.0” from Qiagen (Valencia, Calif.); the “Human siGENOME siRNA Library-Genome” from Dharmacon, Inc. (Lafayette, Colo.); and the Silencer® Human Genome siRNA Library from Ambion (Austin, Tex.). Methods and reagents for introducing siRNAs include, but are not limited to, commercial reagents such as Lipofectamine™ RNAiMAX (Invitrogen, Carlsbad, Calif.), TransMessenger Transfection Reagent (Qiagen, Valencia, Calif.), or Dharma FECT® (Dharmacon, Lafayette, Colo.). See, e.g., Krausz (2007), Mol. Biosyst., 3(4):232-240. In some embodiments, a viral RNAi library is used as described in, e.g., Root et al., (2006), Nat. Methods, 3(9):715-719.

Optionally, the induction test agents to be screened are small molecules. The test molecules may be individual small molecules of choice or in some cases, the small molecule test agents to be screened come from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Indeed, theoretically, the systematic, combinatorial mixing of 100 interchangeable chemical building blocks results in the synthesis of 100 million tetrameric compounds or 10 billion pentameric compounds. See, e.g., Gallop et al., (1994), J. Med. Chem., 37(9), 1233-1251. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al., (1993), Proc. Natl. Acad. Sci. U.S.A., 90:6909-6913; analogous organic syntheses of small compound libraries, as described in Chen et al., (1994), J. Amer. Chem. Soc., 116:2661-2662; Oligocarbamates, as described in Cho, et al., (1993), Science, 261:1303-1305; peptidyl phosphonates, as described in Campbell et al., (1994), J. Org. Chem., 59: 658-660; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.)/.

Recombinant Mammalian Cells Suitable for Selection

Recombinant mammalian cells suitable for selection methods as described herein increase the efficiency of obtaining iPS cells, allow for maintenance of pluripotency of induced pluripotent stem cells, and facilitate the maintenance of a differentiated phenotype for cells differentiated from iPS cells. Accordingly, in some cases, a recombinant mammalian cell suitable for selection methods described herein includes one or more exogenous nucleic acids encoding: (i) an induction factor; and (ii) a selection marker; where expression of the encoded induction factor and the encoded selection marker is driven by the same type of promoter.

In some cases, at least one of the one or more exogenous nucleic acids includes a promoter containing a retroviral silencing element (e.g., an MoMLV LTR promoter), as described herein. Typically, at least one of the one or more exogenous nucleic acids is integrated in the genome of the recombinant mammalian cell.

The nucleic acid sequence encoding the induction factor, and the nucleic acid sequence encoding the selection marker may be on the same expression vector on separate expression vectors. The exogenous nucleic acid sequence encoding the induction factor, and the exogenous nucleic acid sequence encoding the selection marker may be in the same expression cassette or in separate expression cassettes. Where the exogenous nucleic acid sequence encoding the induction factor and the nucleic acid sequence encoding the selection marker are in the same expression cassette, the expression cassette may include an IRES element operably linking the exogenous nucleic acid sequences encoding the induction factor and selection marker. In some cases, where the first and second promoters are the same type of promoter, the type of promoter is a ligand-regulated promoter. Suitable ligand-regulated promoters include promoters comprising, e.g., a tetracycline-responsive element (TRE), an echdysone-responsive element (ERE), or a mifepristone-responsive element (MRE).

In other embodiments, a recombinant mammalian cell suitable for selection methods described herein includes (i) one or more exogenous induction factors (e.g., an Oct4 polypeptide comprising a PTD), or one or more exogenous nucleic acids encoding one or more induction factors, and (ii) a genomically integrated exogenous nucleic acid comprising an open reading frame encoding a selection marker and operably linked to a promoter comprising one or more retroviral silencing elements (e.g., an MoMLV LTR promoter).

The foregoing recombinant mammalian cells suitable for selection may be, e.g., human, non-human primate, mouse, rat, rabbit, sheep, or pig. In some cases, the recombinant mammalian cells are human recombinant cells, e.g., fibroblasts, keratinocytes, lymphocytes, neural progenitor cells, hepatocytes, cardiomyocytes, pancreatic cells, gastric epithelial cells, neurons, or pluripotent stem cells. In some cases, the human recombinant cells are post-natal human recombinant cells. In other cases, the human recombinant cells are fetal human recombinant cells. The recombinant mammalian cell may include exogenous nucleic acids encoding 1, 2, 3, or 4 induction factors. In some embodiments, the one or more exogenous nucleic acids of the recombinant cell encode one, two, three, four, or more of Oct4, Klf4, Sox2, c-Myc, Nanog, or Lin-28 polypeptides. The recombinant mammalian cell may include exogenous nucleic acids encoding 1, 2, 3, 4, or 5 of the above-described selection markers suitable for the selection methods described herein. In some cases, the one or more exogenous nucleic acids encode one, two, three, four, or five of HSV-thymidine kinase, puromycin-N-acetyltransferase, hygromycin phosphotransferase, neomycin phosphotransferase, blasticidin-S-deaminase, a luciferase, or a fluorescent protein. In some embodiments, an encoded selection marker may be a multifunctional polypeptide that comprises the amino acid sequences of more that one selection marker polypeptide, as described above. In some embodiments, where a selection marker contains the amino acid sequences of two selection marker polypeptides (e.g., HSV-thymidine kinase and EGFP), the amino acid sequences may be linked to each other by an intervening 2A peptide sequence of the foot-and-mouth disease virus (F2A) or 2A-like sequences from other viruses. In one embodiment, the encoded multifunctional polypeptide comprises the amino acid sequence of fluorescent timer and HSV-thymidine kinase. In some embodiments, the recombinant mammalian cell comprises first, second, and third nucleic acids each of which comprise a nucleic acid sequence encoding a distinct induction factor operably linked by an IRES element to a nucleic acid sequence encoding a selection marker. In one embodiment, the encoded induction factors are Oct4, Sox2, and Klf4 polypeptides, and the selection marker is HSV-thymidine kinase. In another embodiment, the encoded induction factors are Oct4, Sox2, Klf4, and c-Myc polypeptides, and the selection marker is HSV-thymidine kinase. In other embodiments, each of the encoded induction factors is operably linked by an IRES element to a different selection marker, but at least one of the encoded induction factors comprises the amino acid sequence of HSV-thymidine kinase.

Nucleic Acids for Selection of Induced Cells and Induced Pluripotent Stem Cells

Also provided herein are nucleic acids used in the selection methods described herein. In some cases, the nucleic acid includes: (i) a promoter comprising one or more retroviral silencing elements; (ii) an open reading frame encoding an induction factor; and (iii) an open reading frame encoding a selection marker, where (i) is operably connected to (ii) or (iii), and (ii) and (iii) are operably linked to each other by an IRES sequence.

In some cases, the ORF of the induction factor is 5′ relative to the IRES sequence and the selection marker ORF is 3′ relative to the IRES sequence. In other cases, the configuration of the induction factor ORF and the selection marker ORF are reversed. Suitable silenceable promoters for SEV expression cassettes include those that contain at least one RSE, e.g., the MoMLV LTR promoter. Examples of suitable RSEs include those found in the MoMLV LTR promoter, including, but not limited to, the negative control region (NCR) element, the direct repeat enhancer (DR), and the CpG promoter and primer binding site (PBS).

In other cases, a nucleic acid suitable for use in the selection methods described herein includes: (i) an inducible promoter; (ii) an open reading frame encoding an induction factor; and (iii) an open reading frame encoding a selection marker, wherein (i) is operably connected to (ii) or (iii), and (ii) and (iii) are operably linked to each other by an IRES sequence. In some embodiments, inducible promoters are promoters that are ligand-regulated, e.g., ligand-regulated promoters containing one or more tetracycline-responsive elements, ecdysone-responsive elements, or mifepristone-responsive elements.

In some cases the ORF of the induction factor encodes a polypeptide comprising the amino acid sequence of an Oct4, Sox2, Klf4, or c-Myc polypeptide as described herein. In other cases, the ORF of the induction factor encodes a polypeptide comprising the amino acids sequence of Lin-28 or Nanog.

In some embodiments, the encoded selection marker comprises the amino acid sequence of a selection marker polypeptide suitable for negative selection of induced cells as described above, e.g., an enzyme, a fluorescent protein, or a combination thereof. In some embodiments, the encoded selection marker comprises an enzyme containing an amino acid sequence at least at least 70% to 100%, e.g., 80%, 85%, 90%, 95%, or another percent from about 70% to 100% identical to the amino acid sequence of HSV thymidine kinase, neomycin phosphotransferase, hygromycin phosphotransferase, dihydrofolate reductase, puromycin-N-acetyltransferase, blasticidin-S-deaminase, β-lactamase, alkaline phosphatase, β-galactosidase, cytosine deaminase, carboxypeptidase G2, or a luciferase. In other cases, the encoded selection marker is a fluorescent protein comprising an amino acid sequence at least at least 70% to 100%, e.g., 80%, 85%, 90%, 95%, or another percent from about 70% to 100% identical to the amino acid sequence of EGFP, YFP, Cyan, or dEGFP; monomeric DS-Red, monomeric Orange, and fluorescent timer as described in U.S. Pat. No. 7,217,789. In some cases, the encoded selection marker comprises the amino acid sequence of an enzyme and a fluorescent protein. In some embodiments, where a selection marker contains the amino acid sequences of two selection marker polypeptides (e.g., HSV-thymidine kinase and EGFP), the amino acid sequences may be linked to each other by an intervening 2A peptide sequence of the foot-and-mouth disease virus (F2A) or 2A-like sequences from other viruses. The amino acid sequence of an exemplary 2A peptide sequence is as follows:

| | |
|---|---|
| GSGEGRGSLLTCGDVEFNPGP | (SEQ ID NO: 7) |

In some embodiments, the encoded selection marker comprises the amino acid sequence of HSV-thymidine kinase and a fluorescent polypeptide (e.g., fluorescent timer, EGFP, or d2EGFP).

Negative Selection for Maintenance of Stable iPS Cell Lines or Differentiated Cells Derived from iPS Cells—

Selected cells and cell colonies obtained by any of the selection methods described herein may be subcloned, by any method known in the art, to obtain a pure population of iPS cells, which contains a higher proportion of the iPS cells relative to the total cell population than that found in the total cell population before purification. In some cases, the induced cells are cultured and observed for about 14 days to about 40 days, e.g., 15 days, 16 days, 17 days, 18 days, 19 days, 20 days, 23 days, 24 days, 27 days, 28 days, 29 days, 30 days, 31 days, 33 days, 34 days, 35 days, 36 days, 37 days, 38 days, or other period from about 14 days to about 40 days prior to identifying and selecting clones comprising “induced cells” based on morphological characteristics, as described herein. The induced cells may be cultured in a maintenance culture medium in a 37° C., 5% CO2 incubator, with medium changes about every 1 to 2 days, preferably every day. Examples of maintenance culture media include any and all complete ES media (e.g., MC-ES). The maintenance culture medium may be supplemented with b-FGF or FGF2. In some cases, the maintenance culture medium is supplemented with other factors, e.g., IGF-II or Activin A.

In some embodiments, after washing cell cultures with a physiological buffer, e.g., Hank's balanced salt solution, colonies displaying the morphological characteristics of interest are surrounded by a cloning ring coated with silicone grease on the bottom side. About 100 μl (or 50 μl to 150 μl) of “Detachment Medium For Primate ES Cells” (manufactured by ReproCELL, Tokyo Japan) may be then added to the cloning ring and incubated at 37° C. for about 20 minutes to form a cell suspension. The cell suspension in the ring containing the detached colonies may be added to about 2 ml of MC-ES medium (or other medium described herein), and plated in one well of a MEF-coated 24-well plate or other cell culture vessel of equivalent surface area. After culturing the colony-derived cells in a 5% CO2 (atmospheric O2) cell culture incubator at 37° C. for about 14 hours, the medium is replaced. Subsequently, the medium is replaced about every two days until about 8 days later when a second subculture is carried out.

In some embodiments, in the first subculture, the medium is removed, the cells are washed with Hank's balanced salt solution, and Detachment Medium For Primate ES Cells (ReproCell, Tokyo, Japan) is then added to the cells and incubated at 37° C. for 10 minutes. After the incubation, MC-ES medium (2 ml) is added to the resulting cell suspension to quench the activity of the Detachment Medium. The cell suspension is then transferred to a centrifuge tube, and centrifuged at 200×g at 4° C. for 5 minutes. The supernatant is removed, the cell pellet is resuspended in MC-ES medium, and the resuspended cells are plated on four wells of a MEF-coated 24-well plate and cultured for about seven days until a second subculture is prepared.

In the second subculture, prepared by the method described above, cells are plated on a 60 mm cell culture dish coated with Matrigel™ at a concentration of 20 μg/cm2. About eight days later (approximately 5 weeks after initiating forced expression of IFs), a third subculture is prepared in which cells are plated on two Matrigel™-coated 60 mm cell culture dishes, one of which can subsequently be used for gene expression analysis and the other for continued passaging as described below. One of the subcultures is used for gene expression analysis, as described herein, and the other is passaged as needed to maintain a cell line derived from the induced cell clone.

In some cases, negative selection is also applied during subcloning and passaging of pluripotent stem cell lines obtained by the methods described herein, as this may eliminate any cells that revert to a less potent state that is permissive for selection marker expression due to unsilencing of SEV-mediated expression. In some cases, negative selection of cells may also be applied to cells during or after their differentiation from induced pluripotent stem cell lines, as described herein, so as to eliminate any SEV-bearing cells in which SEV-mediated expression of an induction factor or selection marker becomes unsilenced. While not wishing to be bound by theory, it is believed that ongoing or periodic negative selection of differentiated cells obtained from by differentiation from an induced pluripotent stem cell line will be useful in maintaining a population of cells with a stable differentiated phenotype (e.g., cardiomyocytes, neurons, or hepatocytes), since negative selection will eliminate cells in which exogenous induction factor expression is spontaneously reactivated. This is particularly advantageous in cases where an oncogene such as c-myc is used as an exogenous induction factor, since differentiated induced pluripotent stem cells in which exogenous c-myc expression is reactivated, have a higher potential to induce tumors if transplanted in vivo. Any of the above-described methods for negative selection of induced cells may also be applied to induced pluripotent stem cells or cells differentiated from them.

Accordingly, in some cases a method for maintaining the pluripotency of induced pluripotent stem cells includes depleting a population of induced pluripotent stem cells that express a selection marker polypeptide, where expression of the selection marker polypeptide is driven by a promoter that undergoes transcriptional silencing in pluripotent stem cells.

After subcloning, the induced cells may be subcultured with or without negative selection about every to 7 days. In some cases, the cells are washed with Hank's balanced salt solution, and dispase or Detachment Medium For Primate ES Cells is added, and incubated at 37° C. for 5 to 10 minutes. When approximately more than half of the colonies are detached, MC-ES medium is added to quench enzymatic activity of the detachment medium, and the resulting cell/colony suspension is transferred to a centrifuge tube. Colonies in the suspension are allowed to settle on the bottom of the tube, the supernatant is carefully removed, and MC-ES medium is then added to resuspend the colonies. After examining the size of the colonies, any extremely large ones are broken up into smaller sizes by slow up and down pipetting. Appropriately sized colonies are plated on a matrigel-coated plastic culture dish with a base area of about 3 to 6 times that before subculture. For example, the cells may be split from about 1:6 to about 1:3, e.g., about 1:6, 1:5, 1:4, or 1:3. Where the cells are transgenic for one or more SEVs comprising the ORF of a CLE, the culture media optionally contains an appropriate prodrug substrate for the CLE, e.g., ganciclovir when the CLE is HSV-thymidine kinase.

Examples of culture media useful for culturing human pluripotent stem cells induced from undifferentiated stem cells present in a human post-natal tissue of the present invention include, but are not limited to, the ES medium, and a culture medium suitable for culturing human ES cells such as MEF-conditioned ES medium (MC-ES) or other medium described herein, e.g., mTeSR1™. In some examples, the cells are maintained in the presence of a ROCK inhibitor, as described herein.

### Preparation of iPS Cells

iPS cells may be induced and selected from a wide variety of mammalian cells. Examples of suitable populations of mammalian cells include those that include, but are not limited to: fibroblasts, bone marrow-derived mononuclear cells, skeletal muscle cells, adipose cells, peripheral blood mononuclear cells, macrophages, neural stem cells, hepatocytes, keratinocytes, oral keratinocytes, hair follicle dermal cells, gastric epithelial cells, lung epithelial cells, synovial cells, kidney cells, skin epithelial cells or osteoblasts.

The cells can also originate from many different types of tissue, e.g., bone marrow, skin (e.g., dermis, epidermis), scalp tissue, muscle, adipose tissue, peripheral blood, foreskin, skeletal muscle, or smooth muscle. The cells can also be derived from neonatal tissue, including, but not limited to: umbilical cord tissues (e.g., the umbilical cord, cord blood, cord blood vessels), the amnion, the placenta, or other various neonatal tissues (e.g., bone marrow fluid, muscle, adipose tissue, peripheral blood, skin, skeletal muscle etc.).

The cells can be derived from neonatal or post-natal tissue collected from a subject within the period from birth, including cesarean birth, to death. For example, the tissue may be from a subject who is >10 minutes old, >1 hour old, >1 day old, >1 month old, >2 months old, >6 months old, >1 year old, >2 years old, >5 years old, >10 years old, >15 years old, >18 years old, >25 years old, >35 years old, >45 years old, >55 years old, >65 years old, >80 years old, <80 years old, <70 years old, <60 years old, <50 years old, <40 years old, <30 years old, <20 years old or <10 years old. The subject may be a neonatal infant. In some cases, the subject is a child or an adult. In some examples, the tissue is from a human of age 2, 5, 10 or 20 hours. In other examples, the tissue is from a human of age 1 month, 2 months, 3 months, 4 months, 5 months, 6 months, 9 months or 12 months. In some cases, the tissue is from a human of age 1 year, 2 years, 3 years, 4 years, 5 years, 18 years, 20 years, 21 years, 23 years, 24 years, 25 years, 28 years, 29 years, 31 years, 33 years, 34 years, 35 years, 37 years, 38 years, 40 years, 41 years, 42 years, 43 years, 44 years, 47 years, 51 years, 55 years, 61 years, 63 years, 65 years, 70 years, 77 years, or 85 years old.

The cells may be from non-embryonic tissue, e.g., at a stage of development later than the embryonic stage. In other cases, the cells may be derived from an embryo. In some cases, the cells may be from tissue at a stage of development later than the fetal stage. In other cases, the cells may be derived from a fetus.

The cells are preferably from a human subject but can also be derived from non-human subjects, e.g., non-human mammals. Examples of non-human mammals include, but are not limited to, non-human primates (e.g., apes, monkeys, gorillas), rodents (e.g., mice, rats), cows, pigs, sheep, horses, dogs, cats, or rabbits.

The cells may be collected from subjects with a variety of disease statuses. The cells can be collected from a subject who is free of an adverse health condition. In other cases, the subject is suffering from, or at high risk of suffering from, a disease or disorder, e.g., a chronic health condition such as cardiovascular disease, eye disease (e.g., macular degeneration), auditory disease, (e.g., deafness), diabetes, cognitive impairment, schizophrenia, depression, bipolar disorder, dementia, neurodegenerative disease, Alzheimer's Disease, Parkinson's Disease, multiple sclerosis, osteoporosis, liver disease, kidney disease, autoimmune disease, arthritis, or a proliferative disorder (e.g., a cancer). In other cases, the subject is suffering from, or at high risk of suffering from, an acute health condition, e.g., stroke, spinal cord injury, burn, or a wound. In certain cases, a subject provides cells for his or her future use (e.g., an autologous therapy), or for the use of another subject who may need treatment or therapy (e.g., an allogeneic therapy). In some cases, the donor and the recipient are immunohistologically compatible or HLA-matched.

The cells to be induced can be obtained from a single cell or a population of cells. The population may be homogeneous or heterogeneous. The cells may be a population of cells found in a human cellular sample, e.g., a biopsy or blood sample. Often, the cells are somatic cells. The cells may be a cell line. In some cases, the cells are derived from cells fused to other cells. In some cases, the cells are not derived from cells fused to other cells. In some cases, the cells are not derived from cells artificially fused to other cells. In some cases, the cells are not a cell that has undergone the procedure known as somatic cell nuclear transfer (SCNT) or a cell descended from a cell that underwent SCNT.

The cellular population may include both differentiated and undifferentiated cells. In some cases, the population primarily contains differentiated cells. In other cases, the population primarily contains undifferentiated cells, e.g., undifferentiated stem cells. The undifferentiated cells within the population may be induced to become pluripotent. In some cases, differentiated cells within the cellular population are induced to become pluripotent.

The cellular population may include undifferentiated stem cells or naïve stem cells. In some cases, the undifferentiated stem cells are stem cells that have not undergone epigenetic inactivating modification by heterochromatin formation due to DNA methylation or histone modification of at least four genes, at least three genes, at least two genes, at least one gene, or none of the following: Nanog, Oct4, Sox2 and Tert. Activation, or expression of such genes, e.g., Tert, Nanog, Oct4 or Sox2, may occur when human pluripotent stem cells are induced from undifferentiated stem cells present in a human post-natal tissue.

Collection of Cells

Methods for obtaining human somatic cells are well established, as described in, e.g., Schantz and Ng (2004), A Manual for Primary Human Cell Culture, World Scientific Publishing Co., Pte, Ltd. In some cases, the methods include obtaining a cellular sample, e.g., by a biopsy (e.g., a skin sample), blood draw, or alveolar or other pulmonary lavage. It is to be understood that initial plating densities from of cells prepared from a tissue may be varied based on such variable as expected viablility or adherence of cells from that particular tissue. Methods for obtaining various types of human somatic cells include, but are not limited to, the following exemplary methods:

Bone Marrow

The donor is given a general anesthetic and placed in a prone position. From the posterior border of the ilium, a collection needle is inserted directly into the skin and through the iliac surface to the bone marrow, and liquid from the bone marrow is aspirated into a syringe. The somatic stem cells are enriched by isolating bone marrow cells from an osteogenic zone of bone marrow. A mononuclear cell fraction is then prepared from the aspirate by density gradient centrifugation. The collected crude mononuclear cell fraction is then cultured prior to use in the methods described herein for induction.

Post-Natal Skin

Skin tissue containing the dermis is harvested, for example, from the back of a knee or buttock. The skin tissue is then incubated for 30 minutes at 37° C. in 0.6% trypsin/Dulbecco's Modified Eagle's Medium (DMEM)/F-12 with 1% antibiotics/antimycotics, with the inner side of the skin facing downward.

After the skin tissue is turned over, tweezers are used to lightly scrub the inner side of the skin. The skin tissue is finely cut into 1 mm2 sections using scissors and is then centrifuged at 1200 rpm and room temperature for 10 minutes. The supernatant is removed, and 25 ml of 0.1% trypsin/DMEM/F-12/1% antibiotics, antimycotics, is added to the tissue precipitate. The mixture is stirred at 200-300 rpm using a stirrer at 37° C. for 40 minutes. After confirming that the tissue precipitate is fully digested, 3 ml fetal bovine serum (FBS) (manufactured by JRH) is added, and filtered sequentially with gauze (Type I manufactured by PIP), a 100 μm nylon filter (manufactured by FALCON) and a 40 μm nylon filter (manufactured by FALCON). After centrifuging the resulting filtrate at 1200 rpm and room temperature for 10 minutes to remove the supernatant, DMEM/F-12/1% antibiotics, antimycotics is added to wash the precipitate, and then centrifuged at 1200 rpm and room temperature for 10 minutes. The cell fraction thus obtained is then cultured prior to induction.

Dermal stem cells can be enriched by isolating dermal papilla from scalp tissue. Human scalp tissues (0.5-2 cm or less) are rinsed, trimmed to remove excess adipose tissues, and cut into small pieces. These tissue pieces are enzymatically digested in 12.5 mg/ml dispase (Invitrogen, Carlsbad, Calif.) in DMEM for 24 hours at 4° C. After the enzymatic treatment, the epidermis is peeled off from the dermis; and hair follicles are pulled out from the dermis. Hair follicles are washed with phosphate-buffered saline (PBS); and the epidermis and dermis are removed. A microscope may be used for this procedure. Single dermal papilla derived cells are generated by culturing the explanted papilla on a plastic tissue culture dish in the medium containing DMEM and 10% FCS for 1 week. When single dermal papilla cells are generated, these cells are removed and cultured in FBM supplemented with FGM-2 SingleQuots (Lonza) or cultured in the presence of 20 ng/ml EGF, 40 ng/ml FGF-2, and B27 without serum.

Epidermal stem cells can be also enriched from human scalp tissues (0.5-2 cm2 or less). Human scalp issues is rinsed, trimmed to remove excess adipose tissues, and cut into small pieces. These tissue pieces are enzymatically digested in 12.5 mg/ml dispase (Invitrogen, Carlsbad, Calif.) in Dulbecco's modified Eagle's medium (DMEM) for 24 hours at 4° C. After the enzymatic treatment, the epidermis is peeled off from the dermis; and hair follicles are pulled out from the dermis. The bulb and intact outer root sheath (ORS) are dissected under the microscope. After the wash, the follicles are transferred into a plastic dish. Then the bulge region is dissected from the upper follicle using a fine needle. After the wash, the bulge is transferred into a new dish and cultured in medium containing DMEM/F12 and 10% FBS. After the cells are identified, culture medium is changed to the EpiLife™ Extended-Lifespan Serum-FreeMedium (Sigma).

Post-Natal Skeletal Muscle

After the epidermis of a connective tissue containing muscle such as the lateral head of the biceps brachii muscle or the sartorius muscle of the leg is cut and the muscle tissue is excised, it is sutured. The whole muscle obtained is minced with scissors or a scalpel, and then suspended in DMEM (high glucose) containing 0.06% collagenase type IA and 10% FBS, and incubated at 37° C. for 2 hours.

Cells are collected by centrifugation from the minced muscle, and suspended in DMEM (high glucose) containing 10% FBS. After passing the suspension through a microfilter with a pore size of 40 μm and then a microfilter with a pore size of 20 μm, the cell fraction obtained may be cultured as crude purified cells containing undifferentiated stem cells, and used for the induction of human pluripotent stem cells as described herein.

Post-Natal Adipose Tissue

Cells derived from adipose tissue for use in the present invention may be isolated by various methods known to a person skilled in the art. For example, such a method is described in U.S. Pat. No. 6,153,432, which is incorporated herein in its entirety. A preferred source of adipose tissue is omental adipose tissue. In humans, adipose cells are typically isolated by fat aspiration.

In one method of isolating cells derived from adipose cells, adipose tissue is treated with 0.01% to 0.5%, e.g., 0.04% to 0.2%, 0.1% collagenase; 0.01% to 0.5%, e.g., 0.04%, or 0.2% trypsin; and/or 0.5 ng/ml to 10 ng/ml dispase, or an effective amount of hyaluronidase or DNase (DNA digesting enzyme), and about 0.01 to about 2.0 mM, e.g., about 0.1 to about 1.0 mM, or 0.53 mM ethylenediaminetetraacetic acid (EDTA) at 25 to 50° C., e.g., 33 to 40° C., or 37° C. for 10 minutes to 3 hours, e.g., 30 minutes to 1 hour, or 45 minutes.

Cells are passed through nylon or a cheese cloth mesh filter of 20 microns to 800 microns, more preferably 40 microns to 400 microns, and most preferably 70 microns. Then the cells in the culture medium are subjected to differential centrifugation directly or using Ficoll or Percoll or another particle gradient. The cells are centrifuged at 100 to 3000×g, more preferably 200 to 1500×g, most preferably 500×g for 1 minute to 1 hours, more preferably 2 to 15 minutes and most preferably 5 minutes, at 4 to 50° C., preferably 20 to 40° C. and more preferably about 25° C.

The adipose tissue-derived cell fraction thus obtained may be cultured according to the method described herein as crude purified cells containing undifferentiated stem cells, and used for the induction of human pluripotent stem cells.

Blood

About 50 ml to about 500 ml vein blood or cord blood is collected, and a mononuclear cell fraction is obtained by the Ficoll-Hypaque method, as described in, e.g., Kanof et al., (1993), Current Protocols in Immunology (J. E. Coligan, A. M. Kruisbeek, D. H. Margulies, E. M. Shevack, and W. Strober, eds.), ch. 7.1.1.-7.1.5, John Wiley & Sons, New York).

After isolation of the mononuclear cell fraction, approximately 1×107 to 1×108 human peripheral blood mononuclear cells are suspended in a RPMI 1640 medium containing 10% fetal bovine serum, 100 mg/ml streptomycin and 100 units/ml penicillin, and after washing twice, the cells are recovered. The recovered cells are resuspended in RPMI 1640 medium and then plated in a 100 mm plastic petri dish at a density of about 1×107 cells/dish, and incubated in a 37° C. incubator at 8% CO2. After 10 minutes, cells remaining in suspension are removed and adherent cells are harvested by pipetting. The resulting adherent mononuclear cell fraction is then cultured prior to the induction period as described herein. In some cases, the peripheral blood-derived or cord blood-derived adherent cell fraction thus obtained may be cultured according to the method described herein as crude purified cells containing undifferentiated stem cells, and used for the induction of human pluripotent stem cells.

Macrophages in the peripheral blood can be enriched by culturing the mononuclear cell fraction in low-glucose DMEM supplemented with 10% heat-inactivated fetal bovine serum (FBS; JRH Biosciences, Lenexa, Kans.), 2 mM L-glutamine, 50 U/ml penicillin, and 50 μg/ml streptomycin. In order to expand macrophages, peripheral blood mononuclear cells are spread at a density of 2×106/ml on plastic plates that have been treated with 10 μg/ml FN (Sigma, St. Louis, Mo.) overnight at 4° C. The cells are then cultured without any additional growth factors at 37° C. and 5% CO2 in a humidified atmosphere. The medium containing floating cells is changed every 3 days. Macrophages with observable fibroblastic features may be used for the induction experiments.

In some cases, a cell fraction from peripheral blood, cord blood, or bone marrow is expanded, as described in U.S. patent application Ser. No. 11/885,112, and then used in the induction methods described herein.

Induction of Pluripotent Stem Cells

Overview

During the induction process, forced expression of certain polypeptides, which may include induction factors and selection markers, is carried out in cultured cells for a period of time, after which the induced and selected cells are screened for a number of properties that characterize pluripotent stem cells (e.g., morphological characteristics, gene expression). Induced cells that meet these screening criteria may then be subcloned and expanded. In some cases, the cells to be induced may be cultured for a period of time prior to the induction procedure. Alternatively, the cells to be induced may be used directly in the induction and selection process without a prior culture period. In some cases, different cell culture media are used at different points prior to, during, and after the induction and selection process. For example, one type of culture medium may be used after collection of tissue and/or directly before the induction process, while a second type of media is used during and/or after the induction process. At times, a third type of culture medium is used during and/or after the selection process. The selection process may include one or more rounds of positive selection, negative selection, or both.

Cell Culture

After collection, tissue or cellular samples can be cultured in any medium suitable for the specific cells or tissue collected. Some representative media that the tissue or cells can be cultured in include but are not limited to: multipotent adult progenitor cell (MAPC) medium; FBM (manufactured by Lonza); Embryonic Stem cell (ES) ES medium; Mesenchymal Stem Cell Growth Medium (MSCGM) (manufactured by Lonza); MCDB202 modified medium; Endothelial Cell Medium kit-2 (EBM2) (manufactured by Lonza); Iscove's Modified Dulbecco's Medium (IMDM) (Sigma); Dulbecco's Modified Eagle Medium (DMEM); MEF-conditioned ES (MC-ES); and mTeSR™ (available, e.g., from StemCell Technologies, Vancouver, Canada), See, e.g., Ludwig et al., (2006), Nat. Biotechnol., 24(2):185-187. In other cases, alternative culture conditions for growth of human ES cells are used, as described in, e.g., Skottman et al., (2006), Reproduction, 132(5):691-698.

MAPC (2% FBS) medium may comprise: 60% Dulbecco's Modified Eagle's Medium-low glucose, 40% MCDB 201, Insulin Transferrin Selenium supplement, (0.01 mg/ml insulin; 0.0055 mg/ml transferrin; 0.005 μg/ml sodium selenite), 1× linolenic acid albumin (1 mg/mL albumin; 2 moles linoleic acid/mole albumin), 1 nM dexamethasone, 2% fetal bovine serum, 1 nM dexamethasone, 10⁻⁴ M ascorbic acid, and 10 μg/ml gentamycin

FBM (2% FBS) medium may comprise: MCDB202 modified medium, 2% fetal bovine serum, 5 μg/ml insulin, 50 mg/ml gentamycin, and 50 ng/ml amphotericin-B.

ES medium may comprise: 40% Dulbecco's Modified Eagle's Medium (DMEM) 40% F12 medium, 2 mM L-glutamine, 1× non-essential amino acids (Sigma, Inc., St. Louis, Mo.), 20% Knockout Serum Replacement™ (Invitrogen, Inc., Carlsbad, Calif.), and 10 μg/ml gentamycin.

MC-ES medium may be prepared as follows. ES medium is conditioned on mitomycin C-treated murine embryonic fibroblasts (MEFs), for 20 to 24 hours, harvested, filtered through a 0.45-μM filter, and supplemented with about 0.1 mM β-mercaptoethanol, about 10 ng/ml bFGF or FGF-2, and, optionally, about 10 ng/ml activin A. In some cases, irradiated MEFs are used in place of the mitomycin C-treated MEFs. In other cases, STO (ATCC) or human fibroblast cells are used in place of the MEFs.

Cells may be cultured in medium supplemented with a particular serum. In some embodiments, the serum is fetal bovine serum (FBS). The serum can also be fetal calf serum (FCS). In some cases, the serum may be human serum (e.g., human AB serum). Mixtures of serum may also be used, e.g. mixture of FBS and Human AB, FBS and FCS, or FCS and Human AB.

After collection of tissue and preparation of cells, it may be useful to promote the expansion of tissue stem cells or progenitor cells that may be present among the prepared cells by use of suitable culture conditions. In some cases, a low-serum culture or serum-free medium (as described herein) may facilitate the expansion of tissue stem cells or progenitor cells. Suitable culture media include, but are not limited to, MAPC, FBM, or MSCGM.

Primary culture ordinarily occurs immediately after the cells are isolated from a donor, e.g., human. The cells can also be sub-cultured after the primary culture. A “second” subculture describes primary culture cells subcultured once, a “third” subculture describes primary cultures subcultured twice, a “fourth” subculture describes primary cells subcultured three times, etc. In some cases, the primary cells are subjected to a second subculture, a third subculture, or a fourth subculture. In some cases, the primary cells are subjected to less than four subcultures. The culture techniques described herein may generally include culturing from the period between the primary culture and the fourth subculture, but other culture periods may also be employed. Preferably, cells are cultured from the primary culture to the second subculture. In some cases, the cells may be cultured for about 1 to about 12 days e.g., 2 days, 3 days, 4.5 days, 5 days, 6.5 days, 7 days, 8 days, 9 days, 10 days, or any other number of days from about 1 day to about 12 days prior to undergoing the induction methods described herein. In other cases, the cells may be cultured for more than 12 days, e.g. from about 12 days to about 20 days; from about 12 days to about 30 days; or from about 12 days to about 40 days. In some embodiments, the cells to be induced are passaged four or fewer times (e.g., 3, 2, 1, or 0 times) prior to induction.

In some cases, prior to induction cells are cultured at a low density, e.g., from about 1×10³ cells/cm² to about 1×10⁴ cells/cm². In other cases, prior to induction (e.g., just prior to induction), cells are cultured at a density of about 1×10³ cells/cm² to about 3×10⁴ cells/cm²; or from about 1×10⁴ cells/cm² to about 3×10⁴ cells/cm².

Often the cells and/or tissue are cultured in a first medium, as described above, prior to and/or during the introduction of induction factors to the cells; and then the cells are cultured in a second or third medium during and/or after the introduction of the induction factors to the cells. The second or third medium may be MEF-Conditioned (MC)-ES, mTeSR1™ medium, or other ES cell medium, as described in, e.g., Skottman et al., (2006), Reproduction, 132(5):691-698.

In many examples, the cells are cultured in MAPC, FBM or MSCGM medium prior to the initiation of forced expression of genes or polypeptides in the cells (e.g., immediately after a retroviral infection period); and then, following the initiation of the forced expression, the cells are cultured in MC-ES medium, mTeSR1™ medium, or other ES cell medium as described herein.

Culture of cells may be carried out under low serum culture conditions prior to, during, or following the introduction of induction factors. A “low serum culture condition” refers to the use of a cell culture medium containing a concentration of serum ranging from 0% (v/v) (i.e., serum-free) to about 5% (v/v), e.g., 0% to 2%, 0% to 2.5%, 0% to 3%, 0% to 4%, 0% to 5%, 0.1% to 2%, 0.1% to 5%, 0%, 0.1%, 0.5%, 1%, 1.2%, 1.5%, 2%, 2.5%, 3%, 3.5%, 4%, or 5%. In some embodiments, a low serum concentration is from about 0% (v/v) to about 2% (v/v). In some cases, the serum concentration is about 2%. In other embodiments, cells are cultured under a “high serum condition,” i.e., greater than 5% (v/v) serum to about 20% (v/v) serum, e.g., 6%, 7%, 8%, 10%, 12%, 15%, or 20%. Culturing under high serum conditions may occur prior to, during, and/or after the introduction of induction factors. Media with low concentrations of serum may be particularly useful to enrich undifferentiated stem cells. For example, MSCs are often obtained by isolating the non-hematopoietic cells (e.g., interstitial cells) adhering to a plastic culture dish when tissue, e.g., bone marrow, fat, muscle, or skin etc., is cultured in a culture medium containing a high-concentration serum (5% or more). However, even under these culture conditions, a very small number of undifferentiated cells can be maintained, especially if the cells were passaged under certain culture conditions (e.g., low passage number, low-density culturing or low oxygen).

When either low or high serum conditions are used for culturing the cells, one or more growth factors such as fibroblast growth factor (FGF)-2; basic FGF (bFGF); platelet-derived growth factor (PDGF), epidermal growth factor (EGF); insulin-like growth factor (IGF); IGF II; or insulin can be included in the culture medium. Other growth factors that can be used to supplement cell culture media include, but are not limited to one or more: Transforming Growth Factor β-1 (TGF β-1), Activin A, Noggin, Brain-derived Neurotrophic Factor (BDNF), Nerve Growth Factor (NGF), Neurotrophin (NT)-1, NT-2, or NT-3. In some cases, one or more of such factors is used in place of the bFGF or FGF-2 in the MC-ES medium or other cell culture medium.

The concentration of growth factor(s) (e.g., FGF-2, bFGF, PDGF, EGF, IGF, insulin, IGF II, TGF β-1, Activin A, Noggin, BDNF, NGF, NT-1, NT-2, NT-3) in the culture media described herein (e.g., MAPC, FBM, MC-ES, MSCGM, IMDM, mTeSR1™) may be from about 4 ng/ml to about 50 ng/ml, e.g., about 2 ng/ml, 3 ng/ml, 4 ng/m, 5 ng/ml, 6 ng/ml, 7 ng/ml, 8 ng/ml, 10 ng/ml, 12 ng/ml, 14 ng/ml, 15 ng/ml, 17 ng/ml, 20 ng/ml, 25 ng/ml, 30 ng/ml, 35 ng/ml, 40 ng/ml, 45 ng/ml, or 50 ng/ml. The concentration of growth factors may also be from about 4 ng/ml to about 10 ng/ml; from about 4 ng/ml to about 20 ng/ml; from about 10 ng/ml to about 30 ng/ml; from about 5 ng/ml to about 40 ng/ml; or from about 10 ng/ml to about 50 ng/ml. In other cases, higher concentrations of growth factors may be used, e.g., from about 50 ng/ml to about 100 ng/ml; or from about 50 ng/ml to about 75 ng/ml.

The growth factors may be used alone or in combination. For example, FGF-2 may be added alone to the medium; in another example, both PDGF and EGF are added to the culture medium. Often, growth factors appropriate for a particular cell type may be used. For example, dermal cells may be cultured in the presence of about 20 ng/ml EGF and/or about 40 ng/ml FGF-2, while epidermal cells may be cultured in the presence of about 50 ng/ml EGF and/or 5 ug/ml Insulin.

The induced cells may be maintained in the presence of a rho, or rho-associated, protein kinase (ROCK) inhibitor to reduce apoptosis. A ROCK inhibitor may be particularly useful when the cells are subjected to a harsh treatment, such as an enzymatic treatment. For example, the addition of Y-27632 (Calbiochem; water soluble) or Fasudil (HA1077: Calbiochem), an inhibitor of Rho associated kinase (Rho associated coiled coil-containing protein kinase) may be used to culture the human pluripotent stem cells of the present invention. In some cases the concentration of Y-27632 or Fasudil, is from about 2.5 μM to about 20 μM, e.g., about 2.5 μM, 5 μM, 10 μM, 15 μM, or 20 μM.

The induced cells may be cultured in a maintenance culture medium in a 37° C., 5% CO₂ incubator (e.g., under an atmospheric oxygen level), with medium changes preferably every day. In some embodiments, in order to culture and grow human pluripotent stem cells induced from the undifferentiated stem cells of the present invention present in a human post-natal tissue, it is preferred that the cells are subcultured every 5 to 7 days in a culture medium containing the additives described herein on a MEF-covered plastic culture dish or a matrigel-coated plastic culture dish. Examples of maintenance culture media for induced cells include any and all complete ES cell media (e.g., MC-ES). The maintenance culture medium may be supplemented with b-FGF or FGF2. In some cases, the maintenance culture medium is supplemented with other factors, e.g., IGF-II, Activin A or other growth factor described herein, see, e.g., Bendall et al., (2007), Nature, 30:448(7157):1015-21. In some embodiments, the induced cells are cultured and observed for about 14 days to about 40 days, e.g., 15, 16, 17, 18, 19, 20, 23, 24, 27, 28, 29, 30, 31, 33, 34, 35, 36, 37, 38 days, or other period from about 14 days to about 40 days, prior to identifying and selecting candidate induced pluripotent stem cell colonies based on morphological characteristics.

Morphological characteristics of candidate induced pluripotent stem cell colonies include, but are not limited to, a rounder, smaller cell size relative to surrounding cells and a high nucleus-to-cytoplasm ratio. The size of the candidate induced cell may be from about 5 μm to about 10 μm; from about 5 μm to about 15 μm; from about 5 μm to about 30 μm; from about 10 μm to about 30 μm; or from about 20 μm to about 30 μm. A high nucleus-to-cytoplasm ratio may be from about 1.5:1 to about 10:1, e.g., about 1.5:1; about 2:1; about 3:1; about 4:1; about 5:1; about 7:1; about 8:1; about 9.5:1; or about 10:1. In some cases, the induced cell clones display a flattened morphology relative to mouse ES cells. For example, candidate induced cells derived from peripheral blood cells or from cells cultured in feeder-free media may exhibit a flattened morphology compared to surrounding cells. Another morphological characteristic for identifying induced cell clones is the formation of small monolayer colonies within the space between parental cells (e.g., between fibroblasts).

The induced cells can be plated and cultured directly on tissue culture-grade plastic. Alternatively, cells are plated and cultured on a coated substrate, e.g., a substrate coated with fibronectin, gelatin, Matrigel™ (BD Bioscience), collagen, or laminin. In some cases, untreated petri-dishes may be used. Suitable cell culture vessels include, e.g., 35 mm, 60 mm, 100 mm, and 150 mm cell culture dishes, 6-well cell culture plates, and other size-equivalent cell culture vessels. In some cases, the cells are cultured with feeder cells. For example, the cells may be cultured on a layer, or carpet, of MEFs (e.g., irradiated or mitomycin-treated MEFs).

Typically, the induced cells may be plated (or cultured) at a low density, which may be accomplished by splitting the cells from about 1:8 to about 1:3, e.g., about 1:8; about 1:6; about 1:5; about 1:4; or about 1:3. Cells may be plated at a density of from about 10³ cells/cm² to about 10⁴ cells/cm². In some examples, the cells may be plated at a density of from about 1.5×10³ cells/cm² to about 10⁴ cells/cm²; from about 2×10³ cells/cm² to about 10⁴ cells/cm²; from about 3×10³ cells/cm² to about 10⁴ cells/cm²; from about 4×10³ cells/cm² to about 10⁴ cells/cm²; or from about 10³ cells/cm² to about 9×10³ cells/cm². In some embodiments, the cells may be plated at a density greater than 10⁴ cells/cm², e.g., from about 1.25×10⁴ cells/cm² to about 3×10⁴ cells/cm².

Induction Factors

Inducing a cell to become pluripotent can be accomplished in a number of ways. In some embodiments, the methods for induction of pluripotency in one or more cells include forcing expression of a set of induction factors. Forced expression may include introducing expression vectors encoding polypeptides of interest into cells, introducing exogenous purified polypeptides of interest into cells, or contacting cells with a non-naturally occurring reagent that induces expression of an endogenous gene encoding a polypeptide of interest.

In some cases, the set of IFs includes one or more: an Oct4 polypeptide, a Sox2 polypeptide, a Klf4 polypeptide, or a c-Myc polypeptide. In some cases, the set does not include a c-Myc polypeptide. For example, the set of IFs can include one or more of: an Oct4 polypeptide, a Sox2 polypeptide, and a Klf4 polypeptide, but not a c-Myc polypeptide. In some cases, the set of IFs does not include polypeptides that might increase the risk of cell transformation or the risk of inducing cancer. The ability of c-Myc to induce cell transformation has been described, see, e.g., Adhikary et al., (2005), Nat. Rev. Mol. Cell. Biol., 6(8):635-645.

In some cases, the set includes a c-Myc polypeptide. In certain cases, the c-Myc polypeptide is a constitutively active variant of c-Myc. In some instances, the set includes a c-Myc polypeptide capable of inducible activity, e.g., a c-Myc-ER polypeptide, see, e.g., Littlewood, et al., (1995), Nucleic Acid Res., 23(10):1686-90.

In other cases, the set of IFs includes: an Oct4 polypeptide, a Sox2 polypeptide, and a Klf4 polypeptide,

In some cases, a single IF is used for induction, and is selected from Oct4 or Sox2.

In some cases, the set of IFs includes two IFs, e.g., Oct4 and Sox2.

In some cases, the set of IFs includes three IFs, wherein two of the three IFs are an Oct4 polypeptide and a Sox2 polypeptide. In other cases, the set of IFs includes two IFs, e.g., a c-Myc polypeptide and a Sox2 polypeptide or an Oct4 and a Klf4 polypeptide. In some cases, the set of IFs is limited to Oct4, Sox2, and Klf4 polypeptides. In other cases, the set of IFs may be limited to a set of four IFs: an Oct4 polypeptide, a Sox2 polypeptide, a Klf4 polypeptide, and a c-Myc polypeptide.

A set of IFs may include IFs in addition to an Oct4, a Sox2, and a Klf4 polypeptide. Such additional IFs include, but are not limited to Nanog, TERT, LIN28, CYP26A1, GDF3, FoxD3, Zfp42, Dnmt3b, Ecat1, and Tc11 polypeptides. In some cases, the set of additional IFs does not include a c-Myc polypeptide. In some cases, the set of additional IFs does not include polypeptides that might increase the risk of cell transformation or of inducing cancer.

In some cell types, the expression levels of genes endogenous to the cell are such that one or more IFs as discussed above can be omitted while inducing pluripotency. For example, fibroblasts express c-Myc and Klf4, and it has been demonstrated that exogenous c-Myc is not necessary for the reprogramming of mouse and human fibroblasts (Nakagawa et al., (2008); Wernig et al., (2008) Cell Stem Cell 2, 10-12). Neural progenitor cells, which express Sox2 and c-Myc at levels higher than in ESCs, have been reprogrammed using only Oct4/Klf4 or Oct4/c-Myc (Kim et al., (2008) Nature 454, 646-650). As such, one, two or more factors may be omitted where it has been determined that their expression is dispensable in inducing pluripotency for a given cell type.

Forced expression of IFs may be maintained for a period of at least about 7 days to at least about 40 days, e.g., 8 days, 9 days, 10 days, 11 days, 12 days, 13 days, 14 days, 15 days, 16 days, 17 days, 18 days, 19 days, 20 days, 21 days, 25 days, 30 days, 33 days, or 37 days.

The efficiency of inducing pluripotency in cells of a human population of cells is from at least about 0.001% to at least about 0.1% of the total number of parental cells cultured initially, e.g., 0.002%, 0.0034%, 0.004%, 0.005%, 0.0065%, 0.007%, 0.008%, 0.01%, 0.04%, 0.06%, 0.08%, or 0.09%. At times, depending on the age of the donor, the origin of the tissue, or the culture conditions, higher efficiencies may be achieved.

Forced expression of the IFs may comprise introducing one or more mammalian expression vectors encoding an Oct4, a Sox2, and a Klf4 polypeptide to a population of cells. The IFs may be introduced into the cells as exogenous genes. In some cases, the exogenous genes are integrated into the genome of a host cell and its progeny. In other cases, the exogenous genes persist in an episomal state in the host cell and its progeny. Exogenous genes are genes that are introduced to the cell from an external source. A gene as used herein is a nucleic acid that normally includes an open reading frame encoding a polypeptide of interest, e.g., an IF. The gene preferably includes a promoter operably linked to an open reading frame. In some cases, a natural version of the gene may already exist in the cell but an additional “exogenous gene” is added to the cell to induce polypeptide expression.

The one or more mammalian expression vectors may be introduced into greater than 20% of the total population of cells, e.g., 25%, 30%, 35%, 40%, 44%, 50%, 57%, 62%, 70%, 74%, 75%, 80%, 90%, or other percent of cells greater than 20%. A single mammalian expression vector may contain two or more of the just-mentioned IFs. In other cases, one or more expression vectors encoding an Oct4, Sox2, Klf4, and c-Myc polypeptide are used. In some embodiments, each of the IFs to be expressed is encoded on a separate mammalian expression vector.

In some cases, the IFs are genetically fused in frame with a transport protein amino acid sequence, e.g., that of a VP22 polypeptide as described in, e.g., U.S. Pat. Nos. 6,773,920, 6,521,455, 6,251,398, and 6,017,735. In particular, VP22 polypeptide encompasses polypeptides corresponding to amino acids 60-301 and 159-301 of the full HSV1 VP22 sequence (1-301), whose sequence is disclosed in FIG. 4 in WO 97/05265. Homologous proteins and fragments based on sequences of VP22 protein homologues from other herpes viruses are described in U.S. Pat. No. 6,017,735. Such VP22 sequences confer intercellular transport of VP22 fusion polypeptides from cells that have been transfected with a VP22 fusion polypeptide expression vector to neighboring cells that have not been transfected or transduced. See, e.g., Lemken et al., (2007), Mol Ther., 15(2):310-319. Accordingly, the use of vectors encoding IF-VP22 fusion polypeptides can significantly increase the functional efficiency of transfected mammalian expression vectors in the induction methods described herein.

Examples of suitable mammalian expression vectors include, but are not limited to: recombinant viruses, nucleic acid vectors, such as plasmids, bacterial artificial chromosomes, yeast artificial chromosomes, human artificial chromosomes, transposon vectors, cDNA, cRNA, and PCR product expression cassettes. Examples of suitable promoters for driving expression of IFs include, but are not limited to, retroviral LTR elements; constitutive promoters such as, CMV, CAG, HSV1-TK, SV40, EF-1α, β-actin; PGK, and inducible promoters, such as those containing Tet-operator elements. In some cases, one or more of the mammalian expression vectors encodes, in addition to an IF, a marker gene that facilitates identification or selection of cells that have been transfected or infected. Examples of marker genes include, but are not limited to, genes encoding fluorescent proteins, e.g., EGFP, DS-Red, monomeric Orange, YFP, and CFP; genes encoding proteins conferring resistance to a selection agent, e.g., the neo^{R} gene, and the blasticidin resistance gene.

Recombinant Viruses

Forced expression of an IF may be accomplished by introducing a recombinant virus carrying DNA or RNA encoding an IF to one or more cells. For ease of reference, at times a virus will be referred to herein by the IF it is encoding. For example, a virus encoding an Oct4 polypeptide, may be described as an “Oct4 virus.” In certain cases, a virus may encode more than one copy of an IF or may encode more than one IF, e.g., two IFs, at a time.

Combinations or sets of recombinant viruses may be introduced to the cells for force expression of various sets of IFs. In some cases, the set of IFs expressed by the recombinant viruses includes one or more: an Oct4 polypeptide, a Sox2 polypeptide, a Klf4 polypeptide, or a c-Myc polypeptide. In some cases, the set does not include a c-Myc polypeptide. For example, the set of IFs can include: an Oct4 polypeptide, a Sox2 polypeptide, and a Klf4 polypeptide, but not a c-Myc polypeptide. In some cases, the set of IFs does not include polypeptides that might increase the risk of cell transformation or the risk of inducing cancer. The ability of c-Myc to induce cell transformation has been described, see, e.g., Adhikary et al., (2005), Nat. Rev. Mol. Cell. Biol., 6(8):635-645.

In some cases, the set of IFs to be expressed includes a c-Myc polypeptide. In certain cases, the c-Myc polypeptide is a constitutively active variant of c-Myc. In some instances, the set includes a c-Myc polypeptide capable of inducible activity, e.g., a c-Myc-ER polypeptide, see, e.g., Littlewood, et al., (1995), Nucleic Acid Res., 23(10):1686-90.

In other cases, the set of IFs to be expressed includes: an Oct4 polypeptide, a Sox2 polypeptide, and a Klf4 polypeptide.

In some cases, the set of IFs includes three IFs, wherein two of the three IFs are an Oct4 polypeptide and a Sox2 polypeptide. In other cases, the set of IFs includes two IFs, wherein the two polypeptides are a c-Myc polypeptide and a Sox2 polypeptide. In some cases, the set of IFs is limited to Oct4, Sox2, and Klf4 polypeptides. In other cases, the set of IFs may be limited to a set of four IFs: an Oct4 polypeptide, a Sox2 polypeptide, a Klf4 polypeptide, and a c-Myc polypeptide.

A set of IFs may include IFs in addition to an Oct4, a Sox2, and a Klf4 polypeptide. Such additional IFs include, but are not limited to Lin-28, Nanog, TERT, LIN28, CYP26A1, GDF3, FoxD3, Zfp42, Dnmt3b, Ecat1, and Tc11 polypeptides. In some cases, the set of additional IFs does not include a c-Myc polypeptide. In some cases, the set of additional IFs does not include polypeptides that might increase the risk of cell transformation or of inducing cancer.

Individual viruses may be added to the cells sequentially in time or simultaneously. In some cases, at least one virus, e.g., an Oct4 virus, a Sox2 virus, a Klf4 virus, or a c-Myc virus, is added to the cells at a time different from the time when one or more other viruses are added. In some examples, the Oct4 virus, Sox2 virus and KlF4 virus are added to the cells simultaneously, or very close in time, and the c-Myc virus is added at a time different from the time when the other viruses are added.

At least two recombinant viruses may be added to the cells simultaneously or very close in time. In some examples, Oct4 virus and Sox2 virus are added simultaneously, or very close in time, and the Klf4 virus or c-Myc virus is added at a different time. In some examples, Oct4 virus and Sox2 virus; Oct4 virus and Klf4 virus; Oct4 virus and c-Myc virus; Sox2 virus and Klf4 virus; Sox2 virus and c-Myc virus; or Klf4 and c-Myc virus are added simultaneously or very close in time.

In some cases, at least three viruses, e.g., an Oct4 virus, a Sox2 virus, and a Klf4 virus, are added to the cells simultaneously or very close in time. In other instances, at least four viruses, e.g., Oct4 virus, Sox2 virus, Klf4 virus, and c-Myc virus are added to the cells simultaneously or very close in time.

At times, the efficiency of viral infection can be improved by repetitive treatment with the same virus. In some cases, one or more Oct4 virus, Sox2 virus, Klf4 virus, or c-Myc virus is added to the cells at least two, at least three, or at least four separate times.

Examples of recombinant viruses include, but are not limited, to retroviruses (including lentiviruses); adenoviruses; and adeno-associated viruses. Often, the recombinant retrovirus is murine moloney leukemia virus (MoMLV), but other recombinant retroviruses may also be used, e.g., Avian Leukosis Virus, Bovine Leukemia Virus, Murine Leukemia Virus (MLV), Mink-Cell focus-Inducing Virus, Murine Sarcoma Virus, Reticuloendotheliosis virus, Gibbon Abe Leukemia Virus, Mason Pfizer Monkey Virus, or Rous Sarcoma Virus, see, e.g., U.S. Pat. No. 6,333,195.

In other cases, the recombinant retrovirus is a lentivirus (e.g., Human Immunodeficiency Virus-1 (HIV-1); Simian Immunodeficiency Virus (SIV); or Feline Immunodeficiency Virus (FIV)), See, e.g., Johnston et al., (1999), Journal of Virology, 73(6):4991-5000 (FIV); Nègre D et al., (2002), Current Topics in Microbiology and Immunology, 261:53-74 (SIV); .Naldini et al., (1996), Science, 272:263-267 (HIV).

The recombinant retrovirus may comprise a viral polypeptide (e.g., retroviral env) to aid entry into the target cell. Such viral polypeptides are well-established in the art, see, e.g., U.S. Pat. No. 5,449,614. The viral polypeptide may be an amphotropic viral polypeptide, e.g., amphotropic env, that aids entry into cells derived from multiple species, including cells outside of the original host species. See, e.g., id. The viral polypeptide may be a xenotropic viral polypeptide that aids entry into cells outside of the original host species. See, e.g., id. In some embodiments, the viral polypeptide is an ecotropic viral polypeptide, e.g., ecotropic env, that aids entry into cells of the original host species. See, e.g., id.

Examples of viral polypeptides capable of aiding entry of retroviruses into cells include but are not limited to: MoMLV amphotropic env, MoMLV ecotropic env, MoMLV xenotropic env, vesicular stomatitis virus-g protein (VSV-g), HIV-1 env, Gibbon Ape Leukemia Virus (GALV) env, RD114, FeLV-C, FeLV-B, MLV 10A1 env gene, and variants thereof, including chimeras. See e.g., Yee et al., (1994), Methods Cell Biol., Pt A:99-112 (VSV-G); U.S. Pat. No. 5,449,614. In some cases, the viral polypeptide is genetically modified to promote expression or enhanced binding to a receptor.

In general, a recombinant virus is produced by introducing a viral DNA or RNA construct into a producer cell. In some cases, the producer cell does not express exogenous genes. In other cases, the producer cell is a “packaging cell” comprising one or more exogenous genes, e.g., genes encoding one or more gag, pol, or env polypeptides and/or one or more retroviral gag, pol, or env polypeptides. The retroviral packaging cell may comprise a gene encoding a viral polypeptide, e.g., VSV-g that aids entry into target cells. In some cases, the packaging cell comprises genes encoding one or more lentiviral proteins, e.g., gag, pol, env, vpr, vpu, vpx, vif, tat, rev, or nef. In some cases, the packaging cell comprises genes encoding adenovirus proteins such as E1A or E1B or other adenoviral proteins. For example, proteins supplied by packaging cells may be retrovirus-derived proteins such as gag, pol, and env; lentivirus-derived proteins such as gag, pol, env, vpr, vpu, vpx, vif, tat, rev, and nef; and adenovirus-derived proteins such as E1A and E1B. In many examples, the packaging cells supply proteins derived from a virus that differs from the virus from which the viral vector derives.

Packaging cell lines include but are not limited to any easily-transfectable cell line. Packaging cell lines can be based on 293T cells, NIH3T3, COS or HeLa cell lines. Packaging cells are often used to package virus vector plasmids deficient in at least one gene encoding a protein required for virus packaging. Any cells that can supply a protein or polypeptide lacking from the proteins encoded by such virus vector plasmid may be used as packaging cells. Examples of packaging cell lines include but are not limited to: Platinum-E (Plat-E); Platinum-A (Plat-A); BOSC 23 (ATCC CRL 11554); and Bing (ATCC CRL 11270), see, e.g., Morita et al., (2000), Gene Therapy, 7:1063-1066; Onishi et al., (1996), Experimental Hematology, 24:324-329; U.S. Pat. No. 6,995,009. Commercial packaging lines are also useful, e.g., Ampho-Pak 293 cell line, Eco-Pak 2-293 cell line, RetroPack PT67 cell line, and Retro-X Universal Packaging System (all available from Clontech).

The retroviral construct may be derived from a range of retroviruses, e.g., MoMLV, HIV-1, SIV, FIV, or other retrovirus described herein. The retroviral construct may encode all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides can help facilitate viral entry. In an exemplary embodiment, the recombinant retrovirus is HIV-1 virus comprising a VSV-g polypeptide but not comprising a HIV-1 env polypeptide.

The retroviral construct may comprise: a promoter, a multi-cloning site, and/or a resistance gene. Examples of promoters include but are not limited to CMV, SV40, EF1α, β-actin; retroviral LTR promoters, and inducible promoters. The retroviral construct may also comprise a packaging signal (e.g., a packaging signal derived from the MFG vector; a psi packaging signal). Examples of some retroviral constructs known in the art include but are not limited to: pMX, pBabeX or derivatives thereof. See e.g., Onishi et al., (1996), Experimental Hematology, 24:324-329. In some cases, the retroviral construct is a self-inactivating lentiviral vector (SIN) vector, see, e.g., Miyoshi et al., (1998), J. Virol., 72(10):8150-8157. In some cases, the retroviral construct is LL-CG, LS-CG, CL-CG, CS-CG, CLG or MFG. Miyoshi et al., (1998), J. Virol., 72(10):8150-8157; Onishi et al., (1996), Experimental Hematology, 24:324-329; Riviere et al., (1995), PNAS, 92:6733-6737. Virus vector plasmids (or constructs), include: pMXs, pMXs-IB, pMXs-puro, pMXs-neo (pMXs-IB is a vector carrying the blasticidin-resistant gene in stead of the puromycin-resistant gene of pMXs-puro) Kimatura et al., (2003), Experimental Hematology, 31: 1007-1014; MFG Riviere et al., (1995), Proc. Natl. Acad. Sci. U.S.A., 92:6733-6737; pBabePuro; Morgenstern et al., (1990), Nucleic Acids Research, 18:3587-3596; LL-CG, CL-CG, CS-CG, CLG Miyoshi et al., (1998), Journal of Virology, 72:8150-8157 and the like as the retrovirus system, and pAdex1 Kanegae et al., (1995), Nucleic Acids Research, 23:3816-3821 and the like as the adenovirus system. In exemplary embodiments, the retroviral construct comprises blasticidin (e.g., pMXs-IB), puromycin (e.g., pMXs-puro, pBabePuro); or neomycin (e.g., pMXs-neo). See, e.g., Morgenstern et al., (1990), Nucleic Acids Research, 18:3587-3596.

The retroviral construct may encode one or more IFs. For example, encoded IFs may be operably connected by one or more IRES elements or viral 2a peptide-encoding sequences and expressed as a polycistronic transcript. In addition, two or more of the encoded IF polypeptides may be connected by a viral 2A self-splicing peptide, which allows IFs to be translated as one polypeptide, which is then cleaved into separate polypeptides. See, e.g., Sommer et al, “iPS Cell Generation Using a Single Lentiviral Stem Cell Cassette,” Stem Cells, published online Dec. 18, 2008. In an exemplary embodiment, pMX vectors encoding Oct4, Sox2, Klf4, or c-Myc polypeptides, or variants thereof, are generated or obtained. For example, Oct4 is inserted into pMXs-puro to create pMX-Oct4; Sox2 is inserted into pMXs-neo to create pMX-Sox2; Klf4 is inserted into pMXs-IB to create pMX-Klf4; and c-Myc is inserted into pMXs-IB to create pMX-c-Myc.). In some cases, an expression cassette encodes a polycistronic mRNA (a “polycistronic expression cassette”), which, upon translation gives rise to independent polypeptides comprising different amino acid sequences or functionalities. In some embodiments, a polycistronic expression cassette encodes a “polyprotein” comprising multiple polypeptide sequences that are separated by encoded by a picornavirus, e.g., a foot-and-mouth disease virus (FMDV) viral 2A peptide sequence. The 2A peptide sequence acts co-translationally, by preventing the formation of a normal peptide bond between the conserved glycine and last proline, resulting in ribosome skipping to the next codon, and the nascent peptide cleaving between the Gly and Pro. After cleavage, the short 2A peptide remains fused to the C-terminus of the ‘upstream’ protein, while the proline is added to the N-terminus of the ‘downstream’ protein, which during translation allow cleavage of the nascent polypeptide sequence into separate polypeptides. See, e.g., Trichas et al (2008), BMC Biol, 6:40. Two exemplary 2A nucleotide sequences and their corresponding peptide sequences are shown below:

5′GGCAGTGGAGAGGGCAGAGGAAGTCTGCTAACATGCGGTGACGTCGAGGAGAATCC TGGCCCA3′ (SEQ ID NO:8), which is translated into the peptide sequence:

(SEQ ID NO: 9)
GSGEGRGSLLTCGDVEENPGP;
or
(SEQ ID NO: 10)
5′GGTTCTGGCGTGAAACAGACTTTGAATTTTGACCTTCTCAAGTTGGC
GGGAGACGTGGAGTCCAACCCAGGGCCC3′

which translates to the sequence GSGVKQTNFDLLKLAGDVESNPGP (SEQ ID NO:11)

In other embodiments, a polycistronic expression cassette may incorporate one or more internal ribosomal entry site (IRES) sequences between open reading frames incorporated into the polycistronic expression cassette. IRES sequences and their use are known in the art as exemplified in, e.g., Martinez-Sales, Curr Opin Biotechnol, 10(5):458-464.

Methods of producing recombinant viruses from packaging cells and their uses are well-established, see, e.g, U.S. Pat. Nos. 5,834,256; 6,910,434; 5,591,624; 5,817,491; 7,070,994; and 6,995,009, incorporated herein by reference. Many methods begin with the introduction of a viral construct into a packaging cell line. The viral construct may be introduced by any method known in the art, including but not limited to: the calcium phosphate method (see, e.g., Kokai, Japanese Unexamined Patent Publication No. 2-227075, the lipofection method Felgner et al., (1987), Proc. Natl. Acad. Sci. U.S.A., 84:7413-7417, the electroporation method, microinjection, Fugene transfection, and the like, and any method described herein.

In one example, pMX-Oct4, pMX-Sox2, pMX-Klf4 or pMX-c-Myc is introduced into PlatE cells by Fugene HD (Roche) transfection. The cell culture medium may be replaced with fresh medium comprising FBM (Lonza) supplemented with FGM-2 Single Quots (Lonza). In some embodiments, the medium is replaced from about 12 to about 60 hours following the introduction of the viral construct, e.g., from about 12 to about 18 hours; about 18 to about 24; about 24 to about 30; about 30 to about 36; about 36 to about 42; about 42 to about 48; about 48 to about 54; or about 54 to about 60 hours following introduction of the viral construct to the producer cells. The medium may be replaced from about 24 to about 48 hours after introduction of the viral construct to the producer cells. The supernatant can be recovered from about 4 to about 24 hours following the addition of fresh media, e.g., about 4 hours. In some cases, the supernatant may be recovered about every 4 hours following the addition of fresh media. The recovered supernatant may be passed through a 0.45 uM filter (Millipore). In some cases, the recovered supernatant comprises retrovirus derived from one or more: pMX-Oct4, pMX-Sox2, pMX-Klf4 or pMX-c-Myc.

Adenoviral transduction may be used to force expression of the sets of IFs. Methods for generating adenoviruses and their use are well established as described in, e.g., Straus, The Adenovirus, Plenum Press (NY 1984), 451 496; Rosenfeld, et al., (1991), Science, 252:431-434; U.S. Pat. Nos. 6,203,975, 5,707,618, and 5,637,456. In other cases, adenoviral-associated viral transduction is used to force expression of the sets of IFs. Methods for preparing adeno-associated viruses and their use are well established as described in, e.g., U.S. Pat. Nos. 6,660,514 and 6,146,874.

In an exemplary embodiment, an adenoviral construct is obtained or generated, wherein the adenoviral construct, e.g., Adeno-X, comprises DNA encoding Oct4, Sox2, Klf4, or c-Myc. An adenoviral construct may be introduced by any method known in the art, e.g., Lipofectamine 2000 (Invitrogen) or Fugene HD (Roche), into HEK 293 cells. In some cases, the method further comprises (1) collecting the cells when they exhibit a cytopathic effect (CPE), such effect occurring from about 10 to about 20 days, e.g., about 11, 13, 14, 15, 18, or 20 days after transfection (2) subjecting the cells to from about 2 to about 5 freeze-thaw cycles, e.g., about 3, (3) collecting the resulting virus-containing liquid; (4) purifying the virus using an adenovirus purification kit (Clontech) and (5) storing the virus at −80° C. In some cases, the titer, or plaque-forming unit (PFU), of the adenoviral stocks is determined using an Adeno-X rapid titer kit (Clontech), as described herein.

The cells can be infected using a wide variety of methods. In some cases, the infection of cells occurs by (1) combining one or more, two or more, three or more, or all four: pMX-Oct4 retrovirus, pMX-Sox2 retrovirus, pMX-Klf4, or pMX-c-Myc to obtain a retrovirus solution (2) supplementing the retrovirus solution with from about 2 ug/ml to about 15 ug/ml Polybrene, e.g., about 2 ug/ml, about 3 ug/ml, about 5 ug/ml, about 7 ug/ml, about 10 ug/ml, about 12 ug/ml, or about 15 ug/ml Polybrene; (3) contacting the retroviral solution with the somatic cells, at a m.o.i. (virus-to-cell ratio) of from about 0.5 m.o.i. to about 10 m.o.i., e.g., about 0.5 m.o.i., about 1 m.o.i., about 2 m.o.i., about 5 m.o.i., about 7.5 m.o.i., or about 10 m.o.i.; (4) allowing the contacting of step (3) to continue at 37° C. from about 2 hours to about 24 hours, e.g., about 2 hours, about 3 hours, about 4 hours, about 5 hours, about 6 hours, about 7 hours, about 9 hours, about 10 hours, about 11 hours, about 12 hours, about 14 hours, about 15 hours, about 16 hours, about 17 hours, about 18 hours, about 19 hours, about 20 hours, about 21 hours, about 22 hours, about 23 hours, or about 24 hours; (5) soon after the contacting of step (4), changing the medium to MC-ES medium, as described herein; and (6) changing the MC-ES medium with fresh medium every 1 to 2 days. In some cases, infection of somatic cells occurs by following steps (1) through (6) described herein, with the added step of pre-incubating the somatic cells for a length of time, e.g., about 48 hours, prior to contacting the cells with the retroviral solution. Such pre-incubation may be necessary when the somatic cell expresses an exogenous receptor that was introduced by viral transduction, transfection, or other method. Thus, in some embodiments, if an adenovirus or lentivirus is used to introduce an exogenous receptor, e.g., mCAT1, to the somatic cell; such cells may need to be cultured for a length of time from at least about 30 hours to at least about 60 hours, e.g., about 30, about 35, about 40, about 48, about 52, about 55, or about 60 hours.

The infection of cells may be accomplished by any method known in the art. e.g., Palsson, B., et al., (1995), WO95/10619; Morling, F. J. et al., (1995), Gene Therapy, 2:504-508; Gopp et al., (2006), Methods Enzymol, 420:64-81. For example, the infection may be accomplished by spin-infection or “spinoculation” methods that involve subjecting the cells to centrifugation during the period closely following the addition of virus to the cells. In some cases, virus may be concentrated prior to the infection, e.g., by ultracentrifugation. In some cases, other technologies may be used to aid or improve entry of retroviruses into the target cell. For example, the retrovirus may be contacted with a liposome or immunoliposome to aid or direct entry into a specific cell type. See, e.g., Tan et al., (2007), Mol. Med. 13(3-4):216-226.

In some cases, the retroviruses used for transduction are VSV-G pseudotyped recombinant retroviruses. In some examples, VSV-G pseudotyped recombinant retrovirus is introduced to cells following the method described herein, except that the timing of the preculturing of the cells may vary.

Nucleic Acid Vectors

Nucleic acid vector transfection (e.g., transient transfection) methods may be used to introduce IFs into human cells. Methods for preparation of transfection-grade nucleic acid expression vectors and transfection methods are well established. See, e.g., Sambrook and Russell (2001), “Molecular Cloning: A Laboratory Manual,” 3rd ed, (CSHL Press); and Current Protocols in Molecular Biology, John Wiley & Sons, N.Y. (2005), 9.1-9.14. Examples of high efficiency transfection efficiency methods include “nucleofection,” as described in, e.g., Trompeter (2003), J Immunol. Methods, 274(1-2):245-256, and in international patent application publications WO2002086134, WO200200871, and WO2002086129, transfection with lipid-based transfection reagents such as Fugene® 6 and Fugene® HD(Roche), DOTAP, and Lipofectamine™ LTX in combination with the PLUS™ (Invitrogen, Carlsbad, Calif.), Dreamfect™ (OZ Biosciences, Marseille, France), GeneJuice™ (Novagen, Madison, Wis.), polyethylenimine (see, e.g., Lungwitz et al., (2005), Eur. J. Pharm. Biopharm., 60(2):247-266), and GeneJammer™ (Stratagene, La Jolla, Calif.), and nanoparticle transfection reagents as described in, e.g., U.S. patent application Ser. No. 11/195,066. In some embodiments, induction factors may be stably transfected into cells by the use of a single vector encoding a set of induction factors (e.g., Oct4, Sox2, Klf4, and c-myc) and including cognate transposon elements for a transposase, e.g., the PiggyBAC transposase or the Sleeping Beauty transposase.

Protein Transduction

The induction methods may use protein transduction to introduce at least one of the IFs directly into cells. In some cases, protein transduction method includes contacting cells with a composition containing a carrier agent and at least one purified polypeptide comprising the amino acid sequence of one of the above-mentioned IFs. Examples of suitable carrier agents and methods for their use include, but are not limited to, commercially available reagents such as Chariot™ (Active Motif, Inc., Carlsbad, Calif.) described in U.S. Pat. No. 6,841,535; Bioport® (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents as described in, e.g., in U.S. patent application Ser. No. 10/138,593.

The protein transduction method may comprise contacting a cells with at least one purified polypeptide comprising the amino acid sequence of one of the above-mentioned IFs fused to a protein transduction domain (PTD) sequence (IF-PTD fusion polypeptide). The PTD domain may be fused to the amino terminal of an IF sequence; or, the PTD domain may be fused to the carboxy terminal of an IF sequence. In some cases, the IF-PTD fusion polypeptide is added to cells as a denatured polypeptide, which may facilitate its transport into cells where it is then renatured. Generation of PTD fusion proteins and methods for their use are established in the art as described in, e.g., U.S. Pat. Nos. 5,674,980, 5,652,122, and 6,881,825. See also, Becker-Hapak et al., (2003), Curr Protocols in Cell Biol, John Wiley & Sons, Inc. Exemplary PTD domain amino acid sequences include, but are not limited to, any of the following: YGRKKRRQRRR (SEQ ID NO:12); RKKRRQRR (SEQ ID NO:13); YARAAARQARA (SEQ ID NO:14); THRLPRRRRRR (SEQ ID NO:15); and GGRRARRRRRR (SEQ ID NO:16). Examples of transducible Sox2 and Oct4 polypeptides are described in, e.g., Bosnali et al (2008), Biol Chem, 389(7):851-861.

In some cases, individual purified IF polypeptides are added to cells sequentially at different times. In other embodiments, a set of at least three purified IF polypeptides, but not a purified c-Myc polypeptide, e.g., an Oct4 polypeptide, a Sox2 polypeptide, and a Klf4 polypeptide are added to cells. In some embodiments, a set of four purified IF polypeptides, e.g., purified Oct4, Sox2, Klf4, and c-Myc polypeptides are added to cells. In some embodiments, the purified IF polypeptides are added to cells as one composition (i.e., a composition containing a mixture of the IF polypeptides). In some embodiments, cells are incubated in the presence of a purified IF polypeptide for about 30 minutes to about 24 hours, e.g., 1 hours, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours. In some embodiments, protein transduction of cells is repeated with a frequency of about every day to about every 4 days, e.g., every 1.5 days, every 2 days, every 3 days, or any other frequency from about every day to about every four days with the same or different IF polypeptides.

In some cases, the methods described herein utilize protein transduction and expression vector transduction/transfection in any combination to force expression of a set of IFs as described herein. In some embodiments, retroviral expression vectors are used to force expression of Oct4, a Sox2, and a Klf4 polypeptides in cells, and purified c-Myc purified polypeptide is introduced into cells by protein transduction as described herein. HDAC inhibitor treatment can be used in addition to the purified IF polypeptide. In some cases, a set of at least three purified IF polypeptides, but not a purified c-Myc polypeptide, e.g., an Oct4 polypeptide, a Sox2 polypeptide, and a Klf4 polypeptide are added to cells which are also subjected to HDAC inhibitor treatment.

Induction Factors

Described herein are polypeptides comprising the amino acid sequences of IFs used in the induction methods described herein, and exogenous genes encoding such polypeptides. In some embodiments, an IF amino acid sequence is a naturally occurring amino acid sequence, e.g., that of: human or mouse Oct4, human or mouse Sox2, human or mouse Klf4, human or mouse c-Myc, human or mouse Lin-28, or human or mouse Nanog polypeptides. In other embodiments, the amino acid sequence of an IF is a non-naturally occurring amino acid sequence variant of an IF that is, nevertheless, functionally or structurally homologous to an IF amino acid sequence, as described herein.

Evaluating the structural and functional homology of two or polypeptides generally includes determining the percent identity of their amino acid sequences to each other. Sequence identity between two or more amino acid sequences is determined by conventional methods. See, for example, Altschul et al., (1997), Nucleic Acids Research, 25(17):3389-3402; and Henikoff and Henikoff (1982), Proc. Natl. Acad. Sci. USA, 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art will appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of another peptide. The FASTA algorithm is described by Pearson and Lipman (1988), Proc. Nat'l Acad. Sci. USA, 85:2444, and by Pearson (1990), Meth. Enzymol., 183:63. Briefly, FASTA first characterizes sequence similarity by identifying regions shared by the query sequence (e.g., any of SEQ ID NOs: 6-13) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch (1970), J. Mol. Biol., 48:444-453; Sellers (1974), SIAM J. Appl. Math., 26:787), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson (1990), Meth. Enzymol., 183:63.

Also described herein are nucleic acids (e.g., exogenous genes) encoding Oct4, Sox2, Klf4, or c-Myc polypeptides, as described herein, that hybridize specifically under low, medium, or high stringency conditions to a probe of at least 50 nucleotides from a nucleic acid encoding the amino acid sequence any of the amino acid sequences described herein. Low stringency hybridization conditions include, e.g., hybridization with a 100 nucleotide probe of about 40% to about 70% GC content; at 42° C. in 2×SSC and 0.1% SDS. Medium stringency hybridization conditions include, e.g., at 50° C. in 0.5×SSC and 0.1% SDS. High stringency hybridization conditions include, e.g., hybridization with the above-mentioned probe at 65° C. in 0.2×SSC and 0.1% SDS. Under these conditions, as the hybridization temperature is elevated, a nucleic acid with a higher homology can be obtained. Such nucleic acids encoding Oct4, Sox2, Klf4, or c-Myc polypeptides are useful in the forced expression of these IFs as described herein.

A number of considerations are useful to the skilled artisan in determining if a particular amino acid sequence variant of an IF is suitable for use in the methods described herein. These considerations include, but are not limited to: (1) known structure-function relationships for the IF, e.g., the presence of modular domains such as a DNA binding domain or a transactivation domain, which, in many cases, have been shown to be functionally discrete and capable of independent function; (2) the presence of amino acid sequence conservation among naturally occurring homologs (e.g., in paralogs and orthologs) of the IF, as revealed by sequence alignment algorithms as described herein. Notably, a number of bioinformatic algorithms are known in the art that successfully predict the functional effect, i.e., “tolerance” of particular amino substitutions in the amino acid sequence of a protein on its function. Such algorithms include, e.g., pMUT, SIFT, PolyPhen, and SNPs3D. For a review see, e.g., Ng and Henikoff (2006), Ann Rev Genomics Hum Genet., 7:61-80. For example, pMUT predicts with a high degree of accuracy (about 84% overall) whether a particular amino acid substitution at a given sequence position affects a protein's function based on sequence homology. See Ferrer-Costa et al., (2005), Bioinformatics, 21(14):3176-3178; Ferrer-Costa et al., (2004), Proteins, 57(4):811-819; and Ferrer-Costa et al., (2002), J Mol Biol, 315:771-786. The PMUT algorithm server is publicly available on the world wide web at: //mmb2.pcb.ub.es:8080/PMut/. Thus, for any IF polypeptide amino acid sequence, an “amino acid substitution matrix” can be generated that provides the predicted neutrality or deleteriousness of any given amino acid substitution on IF polypeptide function.

Non-naturally occurring sequence variants can be generated by a number of known methods. Such methods include, but are not limited to, “Gene Shuffling,” as described in U.S. Pat. No. 6,521,453; “RNA mutagenesis,” as described in Kopsidas et al., (2007), BMC Biotechnology, 7:18-29; and “error-prone PCR methods.” Error prone PCR methods can be divided into (a) methods that reduce the fidelity of the polymerase by unbalancing nucleotides concentrations and/or adding of chemical compounds such as manganese chloride (see, e.g., Lin-Goerke et al., (1997), Biotechniques, 23:409-412), (b) methods that employ nucleotide analogs (see, e.g., U.S. Pat. No. 6,153,745), (c) methods that utilize ‘mutagenic’ polymerases (see, e.g., Cline, J. and Hogrefe, H. H. (2000), Strategies (Stratagene Newsletter), 13:157-161 and (d) combined methods (see, e.g., Xu et al., (1999), Biotechniques, 27:1102-1108. Other PCR-based mutagenesis methods include those, e.g., described by Osuna et al., (2004), Nucleic Acids Res., 32(17):e136 and Wong et al., (2004), Nucleic Acids Res., 10;32(3):e26), and others known in the art.

Confirmation of the retention, loss, or gain of function of the amino acid sequence variants of an IF can be determined in various types of assays according to the protein function being assessed. For example, where the IF is a transcriptional activator, e.g., an Oct4, function is readily assessed using cell-based, promoter-reporter assays, where the reporter construct comprises one or more cognate target elements for the transactivator polypeptide to be assayed. Methods for generating promoter-reporter constructs, introducing them into cells, and assaying various reporter polypeptide activities, can be found in detail in, e.g., Current Protocols in Molecular Biology, John Wiley & Sons, N.Y. (2005), 3.16-3.17 and 9.1-9.14, respectively). Promoter activity can be quantified by measuring a property of the reporter polypeptide (e.g., enzymatic activity or fluorescence), reporter polypeptide expression (e.g., by an ELISA assay), or reporter mRNA expression (e.g., by a fluorescent hybridization technique). Suitable reporter polypeptides include, e.g., firefly luciferase, Renilla luciferase, fluorescent proteins (e.g., enhanced green fluorescent protein), β-galactosidase, β lactamase, ALP, and horseradish peroxidase.

For example, luciferase activity can be detected by providing an appropriate luminogenic substrate, e.g., firefly luciferin for firefly luciferase or coelenterazine for Renilla luciferase. Luciferase activity in the presence of an appropriate substrate can be quantified by a number of standard techniques, e.g., luminometry. See, e.g., U.S. Pat. No. 5,744,320. Fluorescent polypeptides (e.g., EGFP) can be detected and quantified in live cells by a number of detection methods known in the art (e.g., fluorimetry or fluorescence microscopy). Details of reporter assay screens in live cells using fluorescent polypeptides, including high-throughput screening methods, can be found, e.g., in U.S. Pat. No. 6,875,578.

Described herein are a number of IFs that are transcriptional activators, i.e., polypeptides that transactivate promoters containing specific target elements to which the transcriptional activator binds as a monomer, a multimer, or in a heteromeric complex with other polypeptides. Naturally occurring transcriptional activators, e.g., Klf4, are modular proteins minimally composed of two domains as follows: a DNA binding domain that dictates the genes to be targeted and an activation domain that governs the nature and the extent of the transcriptional response through interactions with the transcriptional machinery. The two domains typically operate in an independent fashion such that the DNA binding domain of one transcriptional activator, e.g., the DNA binding domain Sox2, can be attached to the transactivation domain of another transcriptional activator, e.g., Herpes VP16, to generate a fully functional, “chimeric” transcriptional activator, e.g., a chimeric Sox2 transcriptional activator as described in, e.g., Kamachi et al., (1999), Mol Cell Biol., 19(1):107-120.

In view of the guidance provided herein, a broad range of IF sequence variants (e.g., Oct4, Sox2, Klf4, or c-Myc sequence variants), operable in the methods described herein, can readily be identified by those of ordinary skill in the art without undue effort.

As referred to herein, an “Oct4 polypeptide” includes human Oct4, mouse Oct4, or any polypeptide that:

(i) includes a DNA binding domain (DBD) that binds to the human nanog gene Octamer element:

5′-TTTTGCAT-3′; and

(ii) is capable of transactivating a promoter comprising one or more nanog Octamer elements. See, e.g., Kuroda et al., (2005), Mol and Cell Biol., 25(6):2475-2485.

In some embodiments, an Oct4 is a polypeptide having the above-mentioned functional properties, and comprising an amino acid sequence at least 70% identical to SEQ ID NO:17 corresponding to the amino acid sequence of human Oct4, also known as Homo sapiens POU class 5 homeobox 1 (POU5F1; GenBank Accession No. NP_{—}002692), e.g., 75%, 80%, 85%, 90%, 91%, 92%, 95%, 97%, 99%, or any other percent identical from at least 70% to 100% identical to SEQ ID NO:17. In some embodiments, an Oct4 is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO: 17, e.g., SEQ ID NO: 17 with at least one amin amino acid substitution, deletion, or insertion. In other embodiments, an Oct-3/4 is a polypeptide having the above-mentioned functional properties comprising the amino acid sequence of SEQ ID NO:17 with up to a total of 30 amino acid substitutions, deletions, insertions, or any combination thereof, e.g., SEQ ID NO:17 with 0, 1, 2, 3, 4, 5, 6, 7, 8, 9, 10, 11, 15, 20, 25, or any other number of amino acid substitutions, deletions, insertions, or any combination thereof, from 0 to 30.

SEQ ID NO: 17 (Human Oct4):
MAGHLASDFAFSPPPGGGGDGPGGPEPGWVDPRTWLSFQGPPGGPGIG
PGVGPGSEVWGIPPCPPPYEFCGGMAYCGPQVGVGLVPQGGLETSQPE
GEAGVGVESNSDGASPEPCTVTPGAVKLEKEKLEQNPEESQDIKALQKE
LEQFAKLLKQKRITLGYTQADVGLTLGVLFGKVFSQTTICRFEALQLSFK
NMCKLRPLLQKWVEEADNNENLQEICKAETLVQARKRKRTSIENRVRGN
LENLFLQCPKPTLQQISHIAQQLGLEKDVVRVWFCNRRQKGKRSSSDYA
QREDFEAAGSPFSGGPVSFPLAPGPHFGTPGYGSPHFTALYSSVPFPE
GEAFPPVSVTTLGSPMHSN

In some embodiments, an Oct4 is a polypeptide having the above-mentioned functional properties, and comprising an amino acid sequence at least 70% identical to SEQ ID NO:18, e.g., 75%, 80%, 85%, 90%, 91%, 92%, 95%, 97%, 99%, or any other percent identical from at least 70% to 100% identical to SEQ ID NO:18, corresponding to amino acids 138-290 of Human Oct4 comprising the highly conserved POU DNA binding domain. In some embodiments, an Oct4 is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:18, e.g., SEQ ID NO:18 with at least one amino acid substitution, deletion, or insertion (e.g., 1 to 10 amino acid substitutions, deletions, or insertions).

SEO ID NO: 18
(POU/DNA Binding Domain of Human Oct4)
DIKALQKELEQFAKLLKQKRITLGYTQADVGLTLGVLFGKVFSQTTIC
RFEALQLSFKNMCKLRPLLQKWVEEADNNENLQEICKAETLVQARKRK
RTSIENRVRGNLENLFLQCPKPTLQQISHIAQQLGLEKDVVRVWFCNR
RQKGKRSSS

Oct4 polypeptides, as described herein, may include naturally occurring or non-naturally occurring homologs of human Oct4. Examples of naturally occurring homologs of human Oct4 include, but are not limited to, those listed under GenBank Accession Nos: NP_{—}002692; NP_{—}001108427; NP_{—}001093427; NP_{—}001009178; and NP_{—}038661, or any other Oct family members that meet the above-mentioned structural and functional criteria.

Examples of non-naturally occurring homologs of human Oct4, include, but are not limited to those described in, e.g., Niwa et al., (2002), Mol Cell Biol., 22(5):1526-1536; and Lunde et al., (2004), Curr. Biol., 14(1):48-55.

Functional assays for the ability of Oct4 polypeptides to bind to the cognate nanog gene octamer element (described above) and to transactivate a promoter containing one or more nanog target elements are known in the art as described in, e.g., Kuroda et al., (supra); and Loh et al., (2006), Nat. Genet., 39(4):431-440.

As referred to herein, a “Sox2 polypeptide” includes human Sox2, mouse Sox2, or any polypeptide that:

(i) includes a DNA binding domain (DBD) that binds to the human nanog gene Sox element:

5′-TACAATG-3′; and

(ii) is capable of transactivating a promoter comprising one or more nanog gene promoter Sox elements. See, e.g., Kuroda et al., (2005), Mol and Cell Biol., 25(6):2475-2485.

In some embodiments, a Sox2 polypeptide is a polypeptide having the above-mentioned functional properties, and comprising the amino acid sequence at least 70% identical to SEQ ID NO:19 corresponding to the amino acid sequence of human Sox2, i.e., sex-determining region Y-box 2 protein (GenBank Accession No. NP_{—}003097), e.g., 75%, 80%, 85%, 90%, 91%, 92%, 95%, 97%, 99%, or any other percent identical from at least 70% to 100% identical to SEQ ID NO:19. In some embodiments, a Sox2 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:19, e.g., SEQ ID NO: 19 with at least one amino acid substitution, deletion, or insertion (e.g., 1 to 10 amino acid substitutions, deletions, or insertions).

In other embodiments, a Sox2 polypeptide is a polypeptide having the above-mentioned functional properties, and comprising the amino acid sequence of SEQ ID NO:19 with up to a total of 30 amino acid substitutions, deletions, insertions, or any combination thereof, e.g., SEQ ID NO:19 with 0, 1, 2, 3, 4, 5, 6, 7, 8, 9, 10, 11, 15, 20, 25, or any other number of amino acid substitutions, deletions, insertions, or any combination thereof, from 0 to 30.

SEQ ID NO: 19 (Human Sox2):
MYNMMETELKPPGPQQTSGGGGGNSTAAAAGGNQKNSPDRVKRPMNAF
MVWSRGQRRKMAQENPKMHNSEISKRLGAEWKLLSETEKRPFIDEAKR
LRALHMKEHPDYKYRPRRKTKTLMKKDKYTLPGGLLAPGGNSMASGVG
VGAGLGAGVNQRMDSYAHMNGWSNGSYSMMQDQLGYPQHPGLNAHGAA
QMQPMHRYDVSALQYNSMTSSQTYMNGSPTYSMSYSQQGTPGMALGSM
GSVVKSEASSSPPVVTSSSHSRAPCQAGDLRDMISMYLPGAEVPEPAA
PSRLHMSQHYQSGPVPGTAINGTLPLSHM

In some embodiments, a Sox2 polypeptide is a polypeptide having the above-mentioned functional properties, and comprising an amino acid sequence at least 70% identical to SEQ ID NO:20, e.g., 75%, 80%, 85%, 90%, 91%, 92%, 95%, 97%, 99%, or any other percent identical from at least 70% to 100% identical to SEQ ID NO:9, amino acids 40-115 of Human Sox2 comprising the highly conserved High Mobility Group-Sox-TCF (HMG-Sox-TCF) motif DNA binding domain (DBD). In some embodiments, a Sox2 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:20, e.g., SEQ ID NO: 20 with at least one amino amino acid substitution, deletion, or insertion (e.g., 1 to 5 amino acid substitutions, deletions, or insertions).

SEQ ID NO: 20 (HMG-Sox2-TCF DBD)
RVKRPMNAFMVWSRGQRRKMAQENPKMHNSEISKRLGAEWKLLSETEK
RPFIDEAKRLRALHMKEHPDYKYRPRRK

Sox2 polypeptides, as described herein, may include naturally occurring or non-naturally occurring homologs of human Sox2. Examples of naturally occurring homologs of human Sox2 include, but are not limited to, those listed under GenBank Accession Nos: NP_{—}001098933; NP_{—}035573, ACA58281; BAA09168; NP_{—}001032751; and NP_{—}648694, or any other Sox family members that meet the above-mentioned structural and functional criteria.

Examples of non-naturally occurring homologs of human Sox2, include, but are not limited to those described in, e.g., Kamachi et al., (1999), Mol Cell Biol., 19(1):107-120.

Functional assays for the ability of Sox2 polypeptides to bind to the nanog gene Sox element and to transactivate a promoter containing one or more nanog Sox elements are known in the art as described in, e.g., Kuroda et al., (supra).

As referred to herein, a “Klf4 polypeptide” includes human Klf4, mouse Klf4, or any polypeptide that:
(i) includes a zinc-finger DNA binding domain (DBD) that binds to a Klf target element, e.g., 5′-GAGGTCC-3′ OR 5′-GGGGTGT-3′; and
(ii) is capable of transactivating a promoter comprising one or more of the above-mentioned target elements. See, e.g., Nakatake et al., (2006), Mol Cell Biol., 24(20):7772-7782.

In some embodiments, a Klf4 polypeptide is a polypeptide having the above-mentioned functional properties, and comprising the amino acid sequence at least 70% identical to SEQ ID NO:21 corresponding to the amino acid sequence of human Klf4, i.e., Kruppel-Like Factor 4 (GenBank Accession No. NP_{—}004226), e.g., 75%, 80%, 85%, 90%, 91%, 92%, 95%, 97%, 99%, or any other percent identical from at least 70% to 100% identical to SEQ ID NO:21. In some embodiments, a Klf4 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:21, e.g., SEQ ID NO:10 with at least one amino acid substitution, deletion, or insertion (e.g., 1 to 10 amino acid substitutions, deletions, or insertions).

In other embodiments, a Klf polypeptide is a polypeptide having the above-mentioned functional properties, and comprising the amino acid sequence of SEQ ID NO:21 with up to a total of 30 amino acid substitutions, deletions, insertions, or any combination thereof, e.g., SEQ ID NO:21 with 0, 1, 2, 3, 4, 5, 6, 7, 8, 9, 10, 11, 15, 20, 25, or any other number of amino acid substitutions, deletions, insertions, or any combination thereof, from 0 to 30.

SEQ ID NO: 21 (Human Klf4):
MAVSDALLPSFSTFASGPAGREKTLRQAGAPNNRWREELSHMKRLPPV
LPGRPYDLAAATVATDLESGGAGAACGGSNLAPLPRRETEEFNDLLDL
DFILSNSLTHPPESVAATVSSSASASSSSSPSSSGPASAPSTCSFTYP
IRAGNDPGVAPGGTGGGLLYGRESAPPPTAPFNLADINDVSPSGGFVA
ELLRPELDPVYIPPQQPQPPGGGLMGKFVLKASLSAPGSEYGSPSVIS
VSKGSPDGSHPVVVAPYNGGPPRTCPKIKQEAVSSCTHLGAGPPLSNG
HRPAAHDFPLGRQLPSRTTPTLGLEEVLSSRDCHPALPLPPGFHPHPG
PNYPSFLPDQMQPQVPPLHYQELMPPGSCMPEEPKPKRGRRSWPRKRT
ATHTCDYAGCGKTYTKSSHLKAHLRTHTGEKPYHCDWDGCGWKFARSD
ELTRHYRKHTGHRPFQCQKCDRAFSRSDHLALHMKRHF

In some embodiments, a Klf4 polypeptide is a polypeptide having the above-mentioned functional properties, and comprising an amino acid sequence at least 70% identical to SEQ ID NO:22, e.g., 75%, 80%, 85%, 90%, 91%, 92%, 95%, 97%, 99%, or any other percent identical from at least 70% to 100% identical to SEQ ID NO:22, amino acids 382-469 of Human Klf4 comprising the highly conserved Zinc Finger motif DNA binding domain (ZF-DBD). In some embodiments, a Klf4 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:22, e.g., SEQ ID NO:22 with at least one amino acid substitution, deletion, or insertion (e.g., 1 to 5 amino acid substitutions, deletions, or insertions).

SEQ ID NO: 22 (Human Klf4-ZF-DBD)
KRTATHTCDYAGCGKTYTKSSHLKAHLRTHTGEKPYHCDWDGCGWKFA
RSDELTRHYRKHTGHRPFQCQKCDRAFSRSDHLALHMKRH

Klf4 polypeptides, as described herein, may include naturally occurring or non-naturally occurring homologs of human Klf4. Examples of naturally occurring homologs of human Klf4 include, but are not limited to, those listed under listed under GenBank Accession Nos: NP_{—}001017280, NP_{—}057354 (Klf2); AAP36222 (Kl5); NP_{—}034767; and NP_{—}446165, or any other Klf family members that meet the above-mentioned structural and functional criteria. Examples of non-naturally occurring Klf4 polypeptides include, but are not limited to, those having the above-mentioned functional properties and comprising an amino acid sequence at least 70%, e.g., 75%, 80%, 85%, 90%, or a percent from 70% to 100% identical to SEQ ID NO:21 or SEQ ID NO:22.

In some embodiments, a Klf4 polypeptide is a non-naturally occurring polypeptide having the above-mentioned functional properties.

Functional assays for the ability of Klf4 polypeptides to bind to any of the above-mentioned target elements and to transactivate a promoter containing one or more of the target elements are known in the art as described in, e.g., Nakatake et al., (supra).

c-Myc Polypeptide

As referred to herein, a “c-Myc polypeptide” includes human c-Myc, mouse c-Myc, or any polypeptide that:

(i) includes a basic helix-loop-helix leucine zipper domain and binds to a target element comprising the sequence: 5′-CACGTG-3′; or 5′-C/GACCACGTGGTG/C-3′ and

(ii) is capable of transactivating a promoter comprising one or more of the above-mentioned target elements. See, e.g., Cowling et al., (2006), Seminars in Canc. Biol., 16:242-252.

In some embodiments, a c-Myc polypeptide is a polypeptide having the above-mentioned functional properties, and comprising an amino acid sequence at least 70% identical to SEQ ID NO:23 corresponding to the amino acid sequence of human c-Myc, i.e., myelocytomatosis viral oncogene homolog (GenBank Accession No. NP_{—}002458), e.g., 75%, 80%, 85%, 90%, 91%, 92%, 95%, 97%, 99%, or any other percent identical from at least 70% to 100% identical to SEQ ID NO:23. In some embodiments, a c-Myc polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:23, e.g., SEQ ID NO:23 with at least one amino acid substitution, deletion, or insertion (e.g., 1 to 10 amino acid substitutions, deletions, or insertions).

In other embodiments, a c-Myc polypeptide is a polypeptide having the above-mentioned functional properties, and comprising the amino acid sequence of SEQ ID NO:23 with up to a total of 30 amino acid substitutions, deletions, insertions, or any combination thereof, e.g., SEQ ID NO:23 with 0, 1, 2, 3, 4, 5, 6, 7, 8, 9, 10, 11, 15, 20, 25, or any other number of amino acid substitutions, deletions, insertions, or any combination thereof, from 0 to 30.

SEQ ID NO: 23 (Human c-Myc):
MDFFRVVENQQPPATMPLNVSFTNRNYDLDYDSVQPYFYCDEEENFYQ
QQQQSELQPPAPSEDIWKKFELLPTPPLSPSRRSGLCSPSYVAVTPFS
LRGDNDGGGGSFSTADQLEMVTELLGGDMVNQSFICDPDDETFIKNII
IQDCMWSGFSAAAKLVSEKLASYQAARKDSGSPNPARGHSVCSTSSLY
LQDLSAAASECIDPSVVFPYPLNDSSSPKSCASQDSSAFSPSSDSLLS
STESSPQGSPEPLVLHEETPPTTSSDSEEEQEDEEEIDVVSVEKRQAP
GKRSESGSPSAGGHSKPPHSPLVLKRCHVSTHQHNYAAPPSTRKDYPA
AKRVKLDSVRVLRQISNNRKCTSPRSSDTEENVKRRTHNVLERQRRNE
LKRSFFALRDQIPELENNEKAPKVVILKKATAYILSVQAEEQKLISEE
DLLRKRREQLKHKLEQLRNSCA

In some embodiments, a c-Myc polypeptide is a polypeptide having the above-mentioned functional properties, and comprising an amino acid sequence at least 70% identical to SEQ ID NO:24, e.g., 75%, 80%, 85%, 90%, 91%, 92%, 95%, 97%, 99%, or any other percent identical from at least 70% to 100% identical to SEQ ID NO:24, amino acids 370-454 of Human c-Myc comprising the highly conserved basic helix-loop-helix (bHLH)-leucine zipper (LZ) DNA binding domain. In some embodiments, a Klf4 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:24, e.g., SEQ ID NO:24 with at least one amino acid substitution, deletion, or insertion (e.g., 1 to 5 amino acid substitutions, deletions, or insertions).

SEQ ID NO: 24 (Human c-Myc bHLH-LZ domain)
KRRTHNVLERQRRNELKRSFFALRDQIPELENNEKAPKVVILKKATAY
ILSVQAEEQKLISEEDLLRKRREQLKHKLEQLRNSCA

c-Myc polypeptides, as described herein, may include naturally occurring or non-naturally occurring homologs of human c-Myc. Examples of naturally occurring homologs of human c-Myc include, but are not limited to, those listed under listed under GenBank Accession Nos: NP_{—}001005154, NP_{—}036735, NP_{—}034979, P0C0N9, and NP_{—}001026123, or any other c-Myc_family members that meet the above-mentioned structural and functional criteria. Examples of non-naturally occurring homologs of human c-Myc include, but are not limited to, those described in, e.g., Chang et al., (2000), Mol Cell Biol., 20:4309-4319.

Functional assays for the ability of c-Myc polypeptides to bind to any of the above-mentioned target elements and to transactivate a promoter containing one or more of the target elements are known in the art as described in, e.g., Gu et al., (1993), Proc. Natl. Acad. Sci. USA, 90:2935-2939.

In some cases, an IF may comprise the amino acid sequence of a polypeptide comprising an amino acid sequence at least 70% (e.g., 75%, 80%, 85%, 90%, 92%, 95%, 98%) or another percent identical from at least 70% to 100% of human or mouse Lin-28:

Human Lin-28 (GenBank NP_078950) Amino Acid
Sequence:
(SEQ ID NO: 25)
MGSVSNQQFAGGCAKAAEEAPEEAPEDAARAADEPQLLHGAGICKWFN
VRMGFGFLSMTARAGVALDPPVDVFVHQSKLHMEGFRSLKEGEAVEFT
FKKSAKGLESIRVTGPGGVFCIGSERRPKGKSMQKRRSKGDRCYNCGG
LDHHAKECKLPPQPKKCHFCQSISHMVASCPLKAQQGPSAQGKPTYFR
EEEEEIHSPTLLPEAQN

Mouse Lin-28 (GenBank NP_665832) Amino Acid
Sequence:
(SEQ ID NO: 26)
MGSVSNQQFAGGCAKAAEKAPEEAPPDAARAADEPQLLHGAGICKWF
NVRMGFGFLSMTARAGVALDPPVDVFVHQSKLHMEGFRSLKEGEAVE
FTFKKSAKGLESIRVTGPGGVFCIGSERRPKGKNMQKRRSKGDRCYN
CGGLDHHAKECKLPPQPKKCHFCQSINHMVASCPLKAQQGPSSQGKP
AYFREEEEEIHSPALLPEAQN

In some cases, an IF may comprise the amino acid sequence of a polypeptide comprising an amino acid sequence at least 70% (e.g., 75%, 80%, 85%, 90%, 92%, 95%, 98%) or another percent identical from at least 70% to 100% of human or mouse Nanog:

Human Nanog (GenBank NP_079141) Amino Acid
Sequence
(SEQ ID NO: 27)
MSVDPACPQSLPCFEASDCKESSPMPVICGPEENYPSLQMSSAEMPH
TETVSPLPSSMDLLIQDSPDSSTSPKGKQPTSAEKSVAKKEDKVPVK
KQKTRTVFSSTQLCVLNDRFQRQKYLSLQQMQELSNILNLSYKQVKT
WFQNQRMKSKRWQKNNWPKNSNGVTQKASAPTYPSLYSSYHQGCLVN
PTGNLPMWSNQTWNNSTWSNQTQNIQSWSNHSWNTQTWCTQSWNNQA
WNSPFYNCGEESLQSCMQFQPNSPASDLEAALEAAGEGLNVIQQTTR
YFSTPQTMDLFLNYSMNMQPEDV

Mouse Nanog (GenBank NP_082292) Amino Acid
Sequence
(SEQ ID NO: 28)
MSVGLPGPHSLPSSEEASNSGNASSMPAVFHPENYSCLQGSATEMLC
TEAASPRPSSEDLPLQGSPDSSTSPKQKLSSPEADKGPEEEENKVLA
RKQKMRTVFSQAQLCALKDRFQKQKYLSLQQMQELSSILNLSYKQVK
TWFQNQRMKCKRWQKNQWLKTSNGLIQKGSAPVEYPSIHCSYPQGYL
VNASGSLSMWGSQTWTNPTWSSQTWTNPTWNNQTWTNPTWSSQAWTA
QSWNGQPWNAAPLHNFGEDFLQPYVQLQQNFSASDLEVNLEATRESH
AHFSTPQALELFLNYSVTPPGEI

Inducing Agents

In some embodiments of the present invention, cells undergoing induction of pluripotency are contacted with inducing agents which can be added to the culture system, i.e., included as additives in the culture medium. Such agents can be biologically active molecules or compounds which increase the efficiency of an aspect of the pluripotency induction process, or are able to substitute for the function of at least one exogenous induction factor (i.e., obviate the need for introducing the exogenous induction factor during induction), which are referred to herein as “induction factor complementing agents.” For example, in some cases, depending on the starting cell type, induction may be achieved by introducing a reduced set of induction factors (e.g., 3, 2, or 1) in combination with one or more inducing agents. Such inducing agents can include, but are not limited to, organic compounds; small molecules; chemokines, cytokines; antisense molecules; antibodies and fragments thereof; genetic agents including, for example, mRNA, shRNA, siRNA,; and the like. Representative examples of such agents are discussed below.

Induction Factor Complementing Agents

In some cases, the use of exogenous Sox2 and/or c-Myc induction factors may be avoided by forcing expression of Oct 4 and Klf4 in combination with BIX-01294 and BayK8644 as described in Shi et al (2008), 3(5):568-574. In other cases, the use of exogenous Klf4 may be avoided by forcing expression of Oct4, Sox2, and c-Myc in the presence of the compound kenpaullone as described in Lyssiotis et al (2009), Proc. Natl. Acad. Sci. USA, e-publication May 15th.

HDAC Inhibitors

Induction of the cells may be accomplished by combining histone deacetylase (HDAC) inhibitor treatment with forced expression of sets of IFs. The cells to be induced may be undifferentiated stem cells present in a human post-natal tissue. In other cases, the cells to be induced are differentiated cells or are a mixture of differentiated and undifferentiated cells.

The HDAC may be combined with the forced expression of a specific set of IFs, e.g., Oct4, Sox2, and Klf4. For example, a human somatic cell is induced to become pluripotent after HDAC inhibitor treatment is combined with forced expression of Oct4, Sox2 and Klf4 or forced expression of Oct4, Sox2, Klf4, and c-Myc. In some cases, human pluripotent stem cells can be induced by introducing three genes (e.g., Oct4, Sox2 and Klf4) or three genes (e.g., Oct4, Sox2 and Klf4) plus the c-Myc gene or a HDAC inhibitor into undifferentiated stem cells present in a human post-natal tissue in which each gene of Tert, Nanog, Oct4 and Sox2 has not undergone epigenetic inactivation. In still other cases, human pluripotent stem cells are induced by introducing three genes (e.g., Oct4, Sox2 and Klf4) or three genes (e.g., Oct4, Sox2 and Klf4) plus the c-Myc gene or a histone deacetylase inhibitor into undifferentiated stem cells after the undifferentiated stem cells were amplified by a primary culture or a second subculture, or a subculture in a low density and subculturing in a culture medium comprising a low-concentration serum.

Cells may be treated with one or more HDACs for about 2 hours to about 5 days, e.g., 3 hours, 6 hours, 12 hours, 14 hours, 18 hours, 1 day, 2 days, 3 days, or 4 days. Treatment with HDAC inhibitor may be initiated prior to beginning forced expression of IFs in the cells. In some cases, HDAC inhibitor treatment begins during or after forced expression of IFs in the cells. In other cases, HDAC inhibitor treatment begins prior to forced expression and is maintained during forced expression.

Suitable concentrations of an HDAC inhibitor range from about 0.001 nM to about 10 mM, depending on the particular HDAC inhibitor to be used, but are selected so as to not significantly decrease cell survival in the treated cells. The HDAC concentration may range from 0.01 nM, to 1000 nM. In some embodiments, the HDAC concentration ranges from about 0.01 nM to about 1000 nM, e.g., about 0.05 nM, 0.1 nM, 0.5 nM, 0.75 nM, 1.0 nM, 1.5 nM, 10 nM, 20 nM, 40 nM, 50 nM, 100 nM, 200 nM, 300 nM, 500 nM, 600 nM, 700 nM, 800 nM, or other concentration from about 0.01 nM to about 1000 nM. Cells are exposed for 1 to 5 days or 1 to 3 days. For example, cells are exposed 1 day, 2 days, 3 days, 4 days or 5 days.

Multiple varieties of HDAC inhibitors can be used for the induction experiments. In a preferred embodiment, the HDAC inhibitor MS-275 is used. Examples of suitable HDAC inhibitors include, but are not limited to, any the following:

Trichostatin A and its analogs, for example: trichostatin A (TSA); and trichostatin C (Koghe et al., (1998), Biochem. Pharmacol., 56:1359-1364).Peptides, for example: oxamflatin [(2E)-5-[3-[(phenylsulfonyl)aminophenyl]-pent-2-ene-4-inohydroxamic acid (Kim et al., (1999), Oncogene, 18:2461-2470); Trapoxin A (cylco-(L-phenylalanyl-L-phenylalanyl-D-pipecolinyl-L-2-amino-8-oxo-9,10-epoxy-decanoyl) (Kijima et al., (1993), J. Biol. Chem., 268:22429-22435); FR901228, depsipeptide (Nakajima et al., (1998). Ex. Cell Res., 241:126-133); FR225497, cyclic tetrapeptide (H. Mori et al., (2000), PCT International Patent Publication WO 00/08048); apicidin, cyclic tetrapeptide [cyclo-(N—O-methyl-L-tryptophanyl-L-isoleucinyl-D-pipecolinyl-L-2-amino-8-oxodecanoyl)] (Darkin-Rattray et al., (1996), Proc. Natl. Acad. Sci. U.S.A., 93:13143-13147; apicidin Ia, apicidin Ib, apicidin Ic, apicidin II, and apicidin IIb (P. Dulski et al., PCT International Patent Publication WO 97/11366); HC-toxin, cyclic tetrapeptide (Bosch et al., (1995), Plant Cell, 7:1941-1950); WF27082, cyclic tetrapeptide (PCT International Patent Publication WO 98/48825); and chlamydocin (Bosch et al., supra). Hybrid polar compounds (HPC) based on hydroxamic acid, for example: salicyl hydroxamic acid (SBHA) (Andrews et al., (2000), International J. Parasitology, 30:761-8); suberoylanilide hydroxamic acid (SAHA) (Richon et al., (1998), Proc. Natl. Acad. Sci. U.S.A., 95: 3003-7); azelaic bishydroxamic acid (ABHA) (Andrews et al., supra); azelaic-1-hydroxamate-9-anilide (AAHA) (Qiu et al., (2000), Mol. Biol. Cell, 11:2069-83); M-carboxy cinnamic acid bishydroxamide (CBHA) (Ricon et al., supra); 6-(3-chlorophenylureido) carpoic hydroxamic acid, 3-C1-UCHA) (Richon et al., supra); MW2796 (Andrews et al., supra); and MW2996 (Andrews et al., supra).Short chain fatty acid (SCFA) compounds, for example: sodium butyrate (Cousens et al., (1979), J. Biol. Chem., 254:1716-23); isovalerate (McBain et al., (1997), Biochem. Pharm., 53:1357-68); valproic acid; valerate (McBain et al., supra); 4-phenyl butyric acid (4-PBA) (Lea and Tulsyan, (1995), Anticancer Research, 15:879-3); phenyl butyric acid (PB) (Wang et al., (1999), Cancer Research 59: 2766-99); propinate (McBain et al., supra); butylamide (Lea and Tulsyan, supra); isobutylamide (Lea and Tulsyan, supra); phenyl acetate (Lea and Tulsyan, supra); 3-bromopropionate (Lea and Tulsyan, supra); tributyrin (Guan et al., (2000), Cancer Research, 60:749-55); arginine butyrate; isobutyl amide; and valproate.Benzamide derivatives, for example: MS-275 [N-(2-aminophenyl)-4-[N-(pyridine-3-yl-methoxycarbonyl)aminomethyl]benzamide] (Saito et al., (1999), Proc. Natl. Acad. Sci. U.S.A., 96:4592-7); and a 3′-amino derivative of MS-275 (Saito et al., supra); and CI-994.

A histone deacetylase inhibitor treatment may be carried out, for example, as follows. The concentration of the HDAC inhibitor may depend on a particular inhibitor, but is preferably 0.001 nM to about 10 mM, and more preferably about 0.01 nM to about 1000 nM. The effective amount or the dosage of a histone deacetylase inhibitor is defined as the amount of the histone deacetylase inhibitor that does not significantly decrease the survival rate of cells, specifically undifferentiated stem cells. Cells are exposed for 1 to 5 days or 1 to 3 days. The exposure period may be less than one day. In a specific embodiment, cells are cultured for about 1 to 5 days, and then exposed to an effective amount of a histone deacetylase inhibitor. However, the histone deacetylase inhibitor may be added at the start of culturing. Within such a time frame, a gene-carrying vehicle such as a vector containing a nucleic acid encoding three genes (Oct4, Sox2 and Klf4) is introduced into cultured cells by a known method.

DNA Demethylating Agents

Induction of the cells may be accomplished according to some embodiments of the present methods by combining treatment with DNA demethylating agents with forced expression of sets of IFs. Methylation contributing to epigenetic inheritance can occur through DNA methylation. DNA methylation in vertebrates typically occurs at CpG (cytosine-phosphate-guanine) sites, which methylation results in the conversion of the cytosine to 5-methylcytosine.

The DNA methyltransferase (DNMT) family of enzymes catalyze the transfer of a methyl group to DNA. The formation of Me-CpG is catalyzed by the DNA methyltransferases such as, i.e., DNMT1, 2 and 3. CpG sites are uncommon in vertebrate genomes but are often found at higher density near vertebrate gene promoters where they are collectively referred to as CpG islands. The methylation state of CpG sites can have a major impact on gene activity/expression. Demethylating agents are compounds that can inhibit methylation of DNA sequences, resulting in the expression of the previously hypermethylated silenced genes. Exemplary DNA demethylating agents include, without limitation, cytidine analogs such as 5-azacytidine (azacitidine) and 5-azadeoxycytidine (decitabine). These compounds work by binding to DNA methyltransferases, the enzymes that catalyze the methylation reaction, which binding titrates out these enzymes to reduce or eliminate activity (Holliday and Ho (2002) Methods 27 (2): 179-83). Both compounds have been approved in the treatment of myelodysplastic syndrome (MDS) by Food and Drug Administration (FDA) in the United States. Azacitidine and decitabine are marketed as Vidaza® and Dacogen® respectively. Azacitidine is approved by the FDA for treating MDS (Issa et al. (2005) Nat Rev Drug Discov 4 (4): 275-6; Gore et al. (2006). “Decitabine” Nat Rev Drug Discov 5 (11): 891-2.)

In some embodiments of the present methods, cells are contacted with one or more methyltransferase inhibitors during exposure of the cells to induction factors. Since hypomethylation is known to induce apoptosis in differentiated cells, whereas embryonic stem cells are resistant (Jackson-Grusby et al. (2001) Nature Genet. 27, 31-39; Lei et al. (1996) Development 122, 3195-3205; Meissner et al. (2005) Nucleic Acids Res. 33, 5868-5877), the methyltransferase inhibitors can be added a sufficient period of time after commencing induction of dedifferentiation such that cytotoxicity is minimized and the frequency of dedifferentiation is maximized. The skilled artisan can readily determine such a time period by performing a time course in which methyltransferase inhibitor is added at various points following induction factor exposure, as is known in the art. An amount of methyltransferase inhibitor effective in improving the efficiency of dedifferentiation can be added, such as about 0.1 μM, generally about 0.5 μM, sometimes 1 μM, or more as needed for the cell type being treated (see, e.g., Mikkelsen, et al. (2008) Nature 454, 49-55).

Alternatively, targeted methods of inhibiting the expression of the methyltransferase inhibitor itself can be employed, such as, for example, by inhibition and/or degradation of the RNA encoding it. An example of modulation of gene expression by target RNA degradation is RNA interference (RNAi). RNAi is a form of antisense-mediated gene silencing involving the introduction of dsRNA leading to the sequence-specific reduction of targeted endogenous mRNA levels. Short interfering RNA (siRNA) molecules of use to this end include a double stranded RNA that comprises about 19 base pairs of a target gene sequence and is capable of inhibiting target gene expression of RNA interference. See, e.g., Scherr et al., (2007), Cell Cycle, 6(4):444-449.

Antisense technology is an effective means for reducing the expression of one or more specific gene products and can therefore prove to be uniquely useful in a number of therapeutic, diagnostic, and research applications. Chemically modified nucleosides are routinely used for incorporation into antisense compounds to enhance one or more properties, such as nuclease resistance, pharmacokinetics or affinity for a target RNA. Generally, the principle behind antisense technology is that an antisense compound hybridizes to a target nucleic acid and effects modulation of gene expression activity or function, such as transcription, translation or splicing.

The modulation of gene expression can also be achieved by, for example, target degradation or occupancy-based inhibition. An example of modulation of RNA target function by degradation is RNase H-based degradation of the target RNA upon hybridization with a DNA-like antisense compound.

In general, their sequence-specificity makes these and other techniques which target the products of genes expressing DNMTs attractive as tools for enhancing the efficiency of pluripotency induction. The use of any such methods as known in the art are envisioned as being of use to the present methods.

TGF-β Receptor Inhibitors

Suitable TGF-β receptor inhibitors for use in the methods described herein include, e.g., in international patent application no. PCT/US10/26451. A number of TGF-β receptor inhibitors are also known in the art and available from commercial sources, e.g., Sigma.

Analysis of Induced Cells

Putative iPS cells and colonies subcultured from those initially identified on the basis of the selection methods described herein may be assayed for any of a number of properties associated with pluripotent stem cells, including, but not limited to, expression of ALP activity, expression of ES cell marker genes, expression of protein markers, hypomethylation of Oct4 and Nanog promoters relative to a parental cells, long term self-renewal, normal diploid karyotype, and the ability to form a teratoma comprising ectodermal, mesodermal, and endodermal tissues.

A number of assays and reagents for detecting ALP activity in cells (e.g., in fixed cells or in living cells) are known in the art. In an exemplary embodiment, colonies to be analyzed are fixed with a 10% formalin neutral buffer solution at room temperature for about 5 minutes, e.g., for 2 to 5 minutes, and then washed with PBS. A chromogenic substrate of ALP, 1 step BCIP (5-Bromo-4-Chloro-3′-Indolyphosphate p-Toluidine Salt) and NBT (Nitro-Blue Tetrazolium Chloride) manufactured by Pierce (Rockford, Ill.) is then added and reacted at room temperature for 20 to 30 minutes. Cells having ALP activity are stained blue-violet.

Putative iPS cell colonies tested for ALP activity may then be assayed for expression of a series of human embryonic stem cell marker (ESCM) genes including, but not limited to, Nanog, TDGF1, Dnmt3b, Zfp42, FoxD3, GDF3, CYP26A1, TERT, Oct4, Sox2, Sall4, and HPRT. See, e.g., Assou et al., (2007), Stem Cells, 25:961-973. Many methods for gene expression analysis are known in the art. See, e.g., Lorkowski et al., (2003), Analysing Gene Expression, A Handbook of Methods: Possibilities and Pitfalls, Wiley-VCH. Examples of suitable nucleic acid-based gene expression assays include, but are not limited to, quantitative RT-PCR (qRT-PCR), microarray hybridization, dot blotting, RNA blotting, RNAse protection, and SAGE.

In some embodiments, levels of ESCM gene mRNA expression levels in putative iPS cell colonies are determined by qRT-PCR. Putative iPS cell colonies are harvested, and total RNA is extracted using the “Recoverall total nucleic acid isolation kit for formaldehyde- or paraformaldehyde-fixed, paraffin-embedded (FFPE) tissues” (manufactured by Ambion, Austin, Tex.). In some instances, the colonies used for RNA extraction are fixed colonies, e.g., colonies that have been tested for ALP activity. The colonies can be used directly for RNA extraction, i.e., without prior fixation. In an exemplary embodiment, after synthesizing cDNA from the extracted RNA, the target gene is amplified using the TaqMan® PreAmp mastermix (manufactured by Applied Biosystems, Foster City, Calif.). Real-time quantitative PCR is performed using an ABI Prism 7900HT using the following PCR primer sets (from Applied Biosystems) for detecting mRNA of the above-mentioned ESCM genes: Nanog, Hs02387400_g1, Dnmt3b, Hs00171876_m1, FoxD3, Hs00255287_s1, Zfp42, Hs01938187_s1, TDGF1, Hs02339499_g1, TERT, Hs00162669_m1, GDF3, Hs00220998_m1, CYP26A1, Hs00175627_m1, GAPDH, Hs99999905_m1).

Putative iPS cell colonies may be assayed by an immunocytochemistry method for expression of protein markers including, but not limited to, SSEA-3, SSEA-4, TRA-1-60, TRA-1-81, CD9, CD24, Thy-1, and Nanog. A wide range of immunocytochemistry assays, e.g., fluorescence immunocytochemistry assays, are known as described in, e.g., Harlow et al., (1988), Antibodies: A Laboratory Manual 353-355, Cold Spring Harbor Laboratory, Cold Spring Harbor, N.Y., and see also, The Handbook—A Guide to Fluorescent Probes and Labeling Technologies (2004), Molecular Probes, Inc., Eugene, Oreg.

In an exemplary embodiment, expression of one or more of the above-mentioned protein markers in putative iPS cell colonies is assayed as follows. Cultured cells are fixed with 10% formaldehyde for 10 min and blocked with 0.1% gelatin/PBS at room temperature for about an hour. The cells are incubated overnight at 4° C. with primary antibodies against SSEA-3 (MC-631; Chemicon), SSEA-4 (MC813-70; Chemicon), TRA-1-60 (ab16288; abcam), TRA-1-81 (ab16289; abcam), CD9 (M-L13; R&D systems), CD24 (ALB9; abcam), Thy1 (5E10; BD Bioscience), or Nanog (MAB1997; R&D Systems). For Nanog staining, cells are permeabilized with 0.1% Triton X-100/PBS before blocking. The cell colonies are washed with PBS three times, then incubated with AlexaFluor 488-conjugated secondary antibodies (Molecular Probes) and Hoechst 33258 (Nacalai) at room temperature for 1 h. After further washing, fluorescence is detected with a fluorescence microscope, e.g., Axiovert 200M microscope (Carl Zeiss).

Methylation Analysis

In some embodiments, a characteristic of the induced cells is reduced methylation of the genomic promoters of Oct4 and Nanog relative to those of their parental cells. Suitable Oct4 promoter regions to be analyzed include, but are not limited to, the Oct4 proximal promoter including conserved region 1 (CR1) and the Oct4 promoter distal enhancer including CR4. Suitable Nanog promoter regions to be analyzed include, but are not limited to, the Nanog proximal promoter including the Oct4 and Sox2 binding sites. See, e.g., Rodda et al., (2005), J Biol. Chem., 280:24731-24737 and Yang et al., (2005), J Cell Biochem., 96:821-830. A number of methods for the quantitative analysis of genomic DNA are known as described in, e.g., Brena et al., (2006), J Mol. Med., 84(5):365-377. In an exemplary embodiment, genomic DNA isolated from putative induced cells and cells used for a comparison is isolated and treated with bisulfite. Bisulfite-treated genomic DNA is then PCR-amplified with primers containing a T7 promoter sequence. Afterwards, RNA transcripts are generated using T7 polymerase and then treated with RNAse A to generate methylation-specific cleavage products. Methylation of individual CpG sites is assessed by MALDI-TOF mass spectrometry of the cleavage products. A detailed description of the method is provided in, e.g., Ehich et al., (2005), Proc. Natl. Acad. Sci. USA, 102:15785-15790.

Self-Renewal Assay

One of the characteristics of stem cells is their ability to proliferate continuously without undergoing senescence. Accordingly, induced cells are assessed for their ability to be passaged continuously in vitro. In some cases, the induced cells are assayed for their ability to be passaged for at least about 30 to at least about 100 times in vitro, e.g., about 33, 35, 40, 45, 51, 56, 60, 68, 75, 80, 90, 93, 100, or any other number of passages from at least about 30 to at least about 100 passages.

In another evaluation, induced cells are assayed for their ability to proliferate for a period of about 30 days to about 500 days from initiation of forced expression of IFs in parental cells, e.g., 40 days, 50 days, 60 days, 70 days, 80 days, 100 days, 150 days, 180 days, 200 days, 250 days, 300 days, 400 days, 450 days or any other period from about 30 days to about 500 days from initiation of forced expression of IFs in the parental cells. In some embodiments, long-term self-renewal of induced cells is determined when the cells are passaged in a defined medium (e.g., mTeSR1 medium) and in the absence of feeder cells, e.g., mTeSR1 medium as described herein. In other embodiments, cells are passaged in MC-ES medium as described herein.

Karyotype Analysis

Induced cells may also be assessed for diploidy and a normal, stable karyotype, e.g., stable after the cells of have been passaged for at least one year in vitro. A number of karotype analysis methods are known in the art. In some embodiments, the karyotype analysis method is multicolor FISH as described in, e.g., Bayani et al., (2004), Curr. Protoc. Cell Biol., Chapter 22:Unit 22.5. In other embodiments, the karyotype analysis includes a molecular karyotype analysis as described in, e.g., Vermeesch et al., (2007), Eur. J. Hum. Genet., 15(11):1105-1114. In an exemplary embodiment, induced cells are pretreated with 0.02 μg/ml colecemid for about 2 to about 3 hours, incubated with about 0.06 to about 0.075M KCl for about 20 minutes, and then fixed with Carnoy's fixative. Afterwards, for multicolor FISH analysis, cells are hybridized with multicolor FISH probes, e.g., those in the Star*FISH© Human Multicolour FISH (M-FISH) Kit from Cambio, Ltd (Cambridge, UK).

Teratoma Analysis

It is generally believed that pluripotent stem cells have the ability to form a teratoma, comprising ectodermal, mesodermal, and endodermal tissues, when injected into an immunocompromised animal. Induced cells or induced pluripotent stem cells (iPS) or ES cell-like pluripotent stem cells may refer to cells having an in vitro long-term self-renewal ability and the pluripotency of differentiating into three germ layers, and said pluripotent stem cells may form a teratoma when transplanted into a test animal such as mouse.

The induced cells may be assessed for pluripotency in a teratoma formation assay in an immunocompromised animal model. The immunocompromised animal may be a rodent that is administered an immunosuppressive agent, e.g., cyclosporin or FK-506. For example, the immunocompromised animal model may be a SCID mouse. About 0.5×10⁶ to about 2.0×10⁶, e.g., 0.6×10⁶, 0.8×10⁶, 1.0×10⁶, 1.2×10⁶, 1.5×10⁶, 1.7×10⁶, or other number of induced cells from about 0.5×10⁶ to about 2.0×10⁶ induced cells/mouse may be injected into the medulla of a testis of a 7-to 8-week-old immunocompromised animal After about 6 to about 8 weeks, the teratomas are excised after perfusing the animal with PBS followed by 10% buffered formalin. The excised teratomas are then subjected to immunohistological analysis. One method of distinguishing human teratoma tissue from host (e.g., rodent) tissue includes immunostaining for the human-specific nuclear marker HuNu. Immunohistological analysis includes determining the presence of ectodermal (e.g., neuroectodermal), mesodermal, and endodermal tissues. Protein markers for ectodermal tissue include, but are not limited to, nestin, GFAP, and integrin β1. Protein markers for mesodermal tissue include, but are not limited to, collagen II, Brachyury, and osteocalcin. Protein markers for endodermal tissue include, but are not limited to, α-fetoprotein (α-FP) and HNF3beta.

Gene Expression

In some embodiments, gene expression analysis is performed on putative iPS cell colonies. Such gene expression analysis may include a comparison of gene expression profiles from a putative iPS cell colony with those of one or more cell types, including but not limited to, (i) parental cells, i.e., one or more cells from which the putative iPS cell colony was induced; (ii) a human ES cell line; or (iii) an established iPS cell line. As known in the art, gene expression data for human ES cell lines are available through public sources, e.g., on the world wide web in the NCBI “Gene Expression Omnibus” database. See, e.g., Barrett et al., (2007), Nuc. Acids Research, D760-D765. Thus, in some embodiments, comparison of gene expression profiles from a putative iPS colony to those of an ES cell line entails comparison experimentally obtained data from a putative iPS cell colony with gene expression data available through public databases. Examples of human ES cell lines for which gene expression data are publicly available include, but are not limited to, hE14 (GEO data set accession numbers GSM151739 and GSM151741), Sheff4 (GEO Accession Nos GSM194307, GSM194308, and GSM193409), h_ES 01 (GEO Accession No. GSM194390), h_ES H9 (GEO Accession No. GSM194392), and h_ES BG03 (GEO Accession No. GSM194391).

It is also possible to accomplish gene expression by analyzing the total RNA isolated from one or more iPS cell lines by a nucleic acid microarray hybridization assay. Examples of suitable microarray platforms for global gene expression analysis include, but are not limited to, the Human Genome U133 plus 2.0 microarray (Affymetrix) and the Whole Human Genome Oligo Micoarray (Agilent). A number of analytical methods for comparison of gene expression profiles are known as described in, e.g., Suarez-Farinas et al., (2007), Methods Mol. Biol., 377:139-152, Hardin et al., (2007), BMC Bioinformatics, 8:220-232, Troyanskaya et al., (2002), Bioinformatics, 18(11):1454-1461, and Knudsen (2002), A Biologist's Guide to Analysis of DNA Microarray Data, John Wiley & Sons. In some embodiments, gene expression data from cells produced by the methods described herein are compared to those obtained from other cell types including, but not limited to, human ES cell lines, parental cells, and multipotent stem cell lines. Suitable statistical analytical metrics and methods include, but are not limited to, the Pearson Correlation, Euclidean Distance, Hierarchical Clustering (See, e.g., Eisen et al., (1998), Proc. Natl. Acad. Sci. USA, 95(25):14863-14868), and Self Organizing Maps (See, e.g., Tamayo et al., (1999), Proc. Natl. Acad. Sci. USA, 96(6):2907-2912.

Cell Differentiation

iPS cells may be differentiated into cell-types of various lineages. Examples of differentiated cells include any differentiated cells from ectodermal (e.g., neurons and fibroblasts), mesodermal (e.g., cardiomyocytes), or endodermal (e.g., pancreatic cells) lineages. The differentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, or cardiomyocytes.

The differentiated cells derived from the induced cells may be terminally differentiated cells, or they may be capable of giving rise to cells of a specific lineage. For example, induced cells can be differentiated into a variety of multipotent cell types, e.g., neural stem cells, cardiac stem cells, or hepatic stem cells. The stem cells may then be further differentiated into new cell types, e.g., neural stem cells may be differentiated into neurons; cardiac stem cells may be differentiated into cardiomyocytes; and hepatic stem cells may be differentiated into hepatocytes.

There are numerous methods of differentiating the induced cells into a more specialized cell type. Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

Any known method of generating neural stem cells from ES cells may be used to generate neural stem cells from induced cells, See, e.g., Reubinoff et al., (2001), Nat, Biotechnol., 19(12):1134-40. For example, neural stem cells may be generated by culturing the induced cells as floating aggregates in the presence of noggin, or other bone morphogenetic protein antagonist, see e.g., Itsykson et al., (2005), Mol, Cell Neurosci., 30(1):24-36. In another example, neural stem cells may be generated by culturing the induced cells in suspension to form aggregates in the presence of growth factors, e.g., FGF-2, Zhang et al., (2001), Nat. Biotech., (19):1129-1133. In some cases, the aggregates are cultured in serum-free medium containing FGF-2. In another example, the induced cells are co-cultured with a mouse stromal cell line, e.g., PA6 in the presence of serum-free medium comprising FGF-2. In yet another example, the induced cells are directly transferred to serum-free medium containing FGF-2 to directly induce differentiation.

Neural stems derived from the induced cells may be differentiated into neurons, oligodendrocytes, or astrocytes. Often, the conditions used to generate neural stem cells can also be used to generate neurons, oligodendrocytes, or astrocytes.

Dopaminergic neurons play a central role in Parkinson's Disease and other neurodegenerative diseases and are thus of particular interest. In order to promote differentiation into dopaminergic neurons, induced cells may be co-cultured with a PA6 mouse stromal cell line under serum-free conditions, see, e.g., Kawasaki et al., (2000) Neuron, 28(1):31-40. Other methods have also been described, see, e.g., Pomp et al., (2005), Stem Cells 23(7):923-30; U.S. Pat. No. 6,395,546, e.g., Lee et al., (2000), Nature Biotechnol., 18:675-679

Oligodendrocytes may also be generated from the induced cells. Differentiation of the induced cells into oligodendrocytes may be accomplished by known methods for differentiating ES cells or neural stem cells into oligodendrocytes. For example, oligodendrocytes may be generated by co-culturing induced cells or neural stem cells with stromal cells, e.g., Hermann et al. (2004), J Cell Sci. 117(Pt 19):4411-22. In another example, oligodendrocytes may be generated by culturing the induced cells or neural stem cells in the presence of a fusion protein, in which the Interleukin (IL)-6 receptor, or derivative, is linked to the IL-6 cyotkine, or derivative thereof. Oligodendrocytes can also be generated from the induced cells by other methods known in the art, see, e.g. Kang et al., (2007) Stem Cells 25, 419-424.

Astrocytes may also be produced from the induced cells. Astrocytes may be generated by culturing induced cells or neural stem cells in the presence of neurogenic medium with bFGF and EGF, see e.g., Brustle et al., (1999), Science, 285:754-756.

Induced cells may be differentiated into pancreatic beta cells by methods known in the art, e.g., Lumelsky et al., (2001) Science, 292:1389-1394; Assady et al., (2001), Diabetes, 50:1691-1697; D'Amour et al., (2006), Nat. Biotechnol., 24:1392-1401; D'Amour et al., (2005), Nat. Biotechnol. 23:1534-1541. The method may comprise culturing the induced cells in serum-free medium supplemented with Activin A, followed by culturing in the presence of serum-free medium supplemented with all-trans retinoic acid, followed by culturing in the presence of serum-free medium supplemented with bFGF and nicotinamide, e.g., Jiang et al., (2007), Cell Res., 4:333-444. In other examples, the method comprises culturing the induced cells in the presence of serum-free medium, activin A, and Wnt protein from about 0.5 to about 6 days, e.g., about 0.5, 1, 2, 3, 4, 5, 6, days; followed by culturing in the presence of from about 0.1% to about 2%, e.g., 0.2%, FBS and activin A from about 1 to about 4 days, e.g., about 1, 2, 3, or 4 days; followed by culturing in the presence of 2% FBS, FGF-10, and KAAD-cyclopamine (keto-N-aminoethylaminocaproyl dihydro cinnamoylcyclopamine) and retinoic acid from about 1 to about 5 days, e.g., 1, 2, 3, 4, or 5 days; followed by culturing with 1% B27, gamma secretase inhibitor and extendin-4 from about 1 to about 4 days, e.g., 1, 2, 3, or 4 days; and finally culturing in the presence of 1% B27, extendin-4, IGF-1, and HGF for from about 1 to about 4 days, e.g., 1, 2, 3, or 4 days.

Hepatic cells or hepatic stem cells may be differentiated from the induced cells. For example, culturing the induced cells in the presence of sodium butyrate may generate hepatocytes, see e.g., Rambhatla et al., (2003), Cell Transplant, 12:1-11. In another example, hepatocytes may be produced by culturing the induced cells in serum-free medium in the presence of Activin A, followed by culturing the cells in fibroblast growth factor-4 and bone morphogenetic protein-2, e.g., Cai et al., (2007), Hepatology, 45(5):1229-39. In an exemplary embodiment, the induced cells are differentiated into hepatic cells or hepatic stem cells by culturing the induced cells in the presence of Activin A from about 2 to about 6 days, e.g., about 2, about 3, about 4, about 5, or about 6 days, and then culturing the induced cells in the presence of hepatocyte growth factor (HGF) for from about 5 days to about 10 days, e.g., about 5, about 6, about 7, about 8, about 9, or about 10 days.

The induced cells may also be differentiated into cardiac muscle cells. Inhibition of bone morphogenetic protein (BMP) signaling may result in the generation of cardiac muscle cells (or cardiomyocytes), see, e.g., Yuasa et al., (2005), Nat. Biotechnol., 23(5):607-11. Thus, in an exemplary embodiment, the induced cells are cultured in the presence of noggin for from about two to about six days, e.g., about 2, about 3, about 4, about 5, or about 6 days, prior to allowing formation of an embryoid body, and culturing the embryoid body for from about 1 week to about 4 weeks, e.g., about 1, about 2, about 3, or about 4 weeks.

In other examples, cardiomyocytes may be generated by culturing the induced cells in the presence of leukemia inhibitory factor (LIF), or by subjecting them to other methods known in the art to generate cardiomyocytes from ES cells, e.g., Bader et al., (2000), Circ. Res., 86:787-794, Kehat et al., (2001), J. Clin. Invest., 108:407-414; Mummery et al., (2003), Circulation, 107:2733-2740.

Examples of methods to generate other cell-types from induced cells include: (1) culturing induced cells in the presence of retinoic acid, leukemia inhibitory factor (LIF), thyroid hormone (T3), and insulin in order to generate adipoctyes, e.g., Dani et al., (1997), J. Cell Sci., 110:1279-1285; (2) culturing induced cells in the presence of BMP-2 or BMP-4 to generate chondrocytes, e.g., Kramer et al., (2000), Mech. Dev., 92:193-205; (3) culturing the induced cells under conditions to generate smooth muscle, e.g., Yamashita et al., (2000), Nature, 408:92-96; (4) culturing the induced cells in the presence of beta-1 integrin to generate keratinocytes, e.g., Bagutti et al., (1996), Dev. Biol., 179:184-196; (5) culturing the induced cells in the presence of Interleukin-3(IL-3) and macrophage colony stimulating factor to generate macrophages, e.g., Lieschke and Dunn (1995), Exp. Hemat., 23:328-334; (6) culturing the induced cells in the presence of IL-3 and stem cell factor to generate mast cells, e.g., Tsai et al., (2000), Proc. Natl. Acad. Sci. USA, 97:9186-9190; (7) culturing the induced cells in the presence of dexamethasone and stromal cell layer, steel factor to generate melanocytes, e.g., Yamane et al., (1999), Dev. Dyn., 216:450-458; (8) co-culturing the induced cells with fetal mouse osteoblasts in the presence of dexamethasone, retinoic acid, ascorbic acid, beta-glycerophosphate to generate osteoblasts, e.g., Buttery et al., (2001), Tissue Eng., 7:89-99; (9) culturing the induced cells in the presence of osteogenic factors to generate osteoblasts, e.g., Sottile et al., (2003), Cloning Stem Cells, 5:149-155; (10) overexpressing insulin-like growth factor-2 in the induced cells and culturing the cells in the presence of dimethyl sulfoxide to generate skeletal muscle cells, e.g., Prelle et al., (2000), Biochem. Biophys. Res. Commun, 277:631-638; (11) subjecting the induced cells to conditions for generating white blood cells; or (12) culturing the induced cells in the presence of BMP4 and one or more: SCF, FLT3, IL-3, IL-6, and GCSF to generate hematopoietic progenitor cells, e.g., Chadwick et al., (2003), Blood, 102:906-915.

In some cases, sub-populations of differentiated cells may be purified or isolated. In some cases, one or more monoclonal antibodies specific to the desired cell type are incubated with the cell population and those bound cells are isolated. In other cases, the desired subpopulation of cells expresses a reporter gene that is under the control of a cell type specific promoter.

In a specific embodiment, the hygromycin B phosphotransferase-EGFP fusion protein is expressed in a cell type specific manner. The method of purifying comprises sorting the cells to select green fluorescent cells and reiterating the sorting as necessary, in order to obtain a population of cells enriched for cells expressing the construct (e.g., hygromycin B phosphotransferase-EGFP) in a cell-type-dependent manner. Selection of desired sub-populations of cells may also be accomplished by negative selection of proliferating cells with the herpes simplex virus thymidine kinase/ganciclovir (HSVtk/GCV) suicide gene system or by positive selection of cells expressing a bicistronic reporter, e.g., Anderson et al. (2007) Mol. Ther. (11):2027-2036.

In general, it is expected that an SEV that has undergone silencing in a pluripotent stem cell will remain silent even after differentiation of the induced pluripotent stem cell into a cell type that is normally permissive for SEV expression. This is supported by the fact that when iPS cells are used to generate chimeric mice, iPS-derived tissues do not generally exhibit reactivation of the genomically integrated viral transgenes. Thus, unsilencing of SEV-dependent selection marker expression, in a differentiated cell following differentiation of an induced pluripotent stem cell may indicate the conversion of the cell to an unstable and/or undesirable, cell phenotype. Accordingly, where induced pluripotent stem cells have been obtained by the selection methods described herein, any of the above-described differentiation protocols may include depleting a population of induced pluripotent stem cells that express a selection marker polypeptide, where expression of the selection marker polypeptide is driven by a promoter that undergoes transcriptional silencing in pluripotent stem cells. In some embodiments, a method for maintaining differentiation of cells differentiated from induced pluripotent stem cells includes depleting the differentiated cells of cells that express a selection marker polypeptide, where expression of the selection marker polypeptide is driven by a promoter that undergoes transcriptional silencing in pluripotent stem cells. Suitable selection markers for maintaining differentiation of cells differentiated from iPS cells include any of the selection markers described herein for use in negative selection, e.g., HSV-thymidine kinase or another conditionally-lethal enzyme, reporter enzymes (e.g., a luciferase or (β-lactamase), or fluorescent proteins (e.g., monomeric DS-red, EGFP, or fluorescent timer).

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### iPSC Derivation using Viral Vector Conditionally Lethal Gene Selection

Murine Moloney retroviral vectors are constructed from pMXs packaging vectors (see, e.g., Kitamura et al., (2003), Exp. Hematol. 31, 1007-1014) encoding human, OCT4, SOX2, KLF4, MYC, or EGFP each followed by an IRES-thymidine kinase (TK) open reading frame. The nucleotide sequences of such vectors (pMXs-Oct4-IRES-TK, pMXs-Sox2-IRES-TK, pMXs-Klf4-IRES-TK, pMXs-c-Myc-IRES-TK, and pMXs-IRES-EGFP) are described in the vector sequence appendix that follows this section. Maps of pMXs-Oct4-IRES-TK and pMXs-Sox2-IRES-TK are shown in FIG. 4. Primary human fibroblast cultures are transduced with a cocktail of retroviral vectors with each vector at a multiplicity of infection of 10 for a period of 24 hours. Four transduction conditions for human iPS cell generation are tested:
1. Transduction with three MoMLV retroviruses containing expression cassettes for human Klf4-IRES-tk, human Sox2-IRES-tk, and human Oct 4-IRES-tk, respectively.
2. Transduction with four retroviruses containing expression cassettes for human Klf4-IRES-tk, human Sox2-IRES-tk, human Oct 4-IRES-tk, and IRES-EGFP, respectively.
3. Transduction with four retroviruses containing expression cassettes for human Klf4-IRES-tk, human Sox2-IRES-tk, human Oct 4-IRES-tk, and human c-Myc-IRES-tk, respectively.
4. Transduction with five retroviruses containing expression cassettes for human Klf4-IRES-tk, human Sox2-IRES-tk, human Oct 4-IRES-tk, human c-Myc-IRES-tk, and IRES-EGFP, respectively.

Approximately 3-5 days after viral transduction, fibroblasts are switched from human fibroblast medium into human ES cell supportive medium and monitored daily by for the appearance of putative iPSC colonies.

Putative iPSC colonies are identified based on morphological criteria (e.g., small round cell shape with high nuclear/somatic ratio as with human ES cells) approximately two weeks after transduction for cultures transduced with a viral cocktail including a c-MYC-encoding virus, and approximately four weeks after transduction with viral cocktails not containing a c-MYC-encoding virus.

Initial putative iPSC colonies are picked and propagated clonally to derive iPSC lines. Each week thereafter EGFP expression is briefly monitored by epifluoresence imaging in colonies originating from cultures transduced with a viral cocktail that included the EGFP retrovirus. After approximately one month, clonally-derived putative iPSC cultures are split into parallel cultures maintained in 5 μM ganciclovir (selected iPSCs) or in the absence of ganciclovir (non-selected iPSCs). It is expected that putative iPSCs that exhibit residual induction factor/thymidine kinase expression will be killed off in the presence of ganciclovir over time due to the conversion of ganciclovir to a cytotoxin by thymidine kinase. Conversely, those putative iPSCs that have undergone complete retroviral silencing, which is associated with full conversion to a pluripotent epigenetic state, will survive and proliferate in the presence of ganciclovir. Further, it is also expected that GFP expression will wane over time and will be a useful indicator of ongoing silencing of the retroviral transgenes. Accordingly, ganciclovir treatment is continued at least until EGFP expression is no longer detected.

Selected and non-selected iPSC lines are compared in a battery of iPSC characterization tests including Q-PCR and immunohistochemistry assays for the expression of alkaline phosphatase, the expression of viral transgenes, expression of pluripotency marker genes, e.g., Nanog and endogenous Oct 4 expression. The ability of the selected iPSC lines to form teratomas in immune-compromised mice is also evaluated as a stringent test of each iPSC line's pluripotency.

Directed differentiation protocols (e.g. for spinal cord motor neurons) are also applied to and compared in selected versus non-selected iPSC lines. It is expected that the differentiation behavior (e.g., the proportion of cells of a given differentiated cell type) of selected iPSC lines is more consistent than that observed for non-selected iPSC lines. Further, it is expected that occasionally in cultures of cells differentiated from non-selected iPSC lines, EGFP⁺, ganciclovir-sensitive, iPSC-like colonies may arise due to spontaneous reactivation of the silenced retroviral transgenes. In contrast, it is expected that cultures of differentiated cells obtained from selected iPSCs will not or will rarely give rise to “revertant”-iPSC-like colonies due to spontaneous retroviral transgene reactivation. However, it may also be necessary to incubate differentiated cell cultures with ganciclovir to completely eliminate the occurrence of reversion to an iPSC phenotype due to transgene reactivation.

Overall, it is expected that negative selection of putative iPSCs in the presence of ganciclovir will allow enrichment of iPSC lines that are fully converted to a stable pluripotent epigenetic state, enable more consistent differentiation of iPSCs to a desired cell type, and avoid spontaneous reversion of differentiated cells to an iPSC state due to reactivation of viral transgenes.

### Example 2

### iPSC Derivation using Viral Vector Conditionally Lethal Protein-Fluoresent Fusion Protein Gene Selection

iPSC induction is performed as described in Example 1, but the negative selection marker gene encodes a fusion protein comprising the amino acid sequence of herpes thymidine kinase (on the N-terminal side) fused to a fluorescent protein such as EGFP or an EGFP variant (see, e.g., Jacobs et al (1999), Neoplasia, 1(2):154-161. Thus, the single fusion protein exhibits TK activity and fluorescence, and therefore there is no need for a separate retrovirus encoding a fluorescent reporter protein. This scheme has the advantage of avoiding the need to transduce cells with an additional virus for expression of a fluorescent reporter protein. In addition, as TK and the fluorescent protein are encoded in the same open reading frame, their protein expression level is identical, whereas this is generally not the case for expression of proteins using a bicistronic-IRES-containing vector. Thus, the level of fluorescence is expected to more faithfully track TK expression and thereby aid in optimizing timing of TK-ganciclovir-based negative selection of putative iPSC clones. The amino acid sequence of an exemplary TK-EGFP fusion polypeptide is shown below:

TK-EGFP:
(SEQ ID NO: 29)
MASYPCHQHASAFDQAARSRGHNNRRTALRPRRQQKATEVRLEQKMP
TLLRVYIDGPHGMGKTTTTQLLVALGSRDDIVYVPEPMTYWRVLGAS
ETIANIYTTQHRLDQGEISAGDAAVVMTSAQITMGMPYAVTDAVLAP
HIGGEAGSSHAPPPALTLIFDRHPIAALLCYPAARYLMGSMTPQAVL
AFVALIPPTLPGTNIVLGALPEDRHIDRLAKRQRPGERLDLAMLAAS
PRLWAACQYGAVSAGRRVVAGGLGTAFGGGRAAPGCRAPEQRGPTTP
YRGHVIYPVSGPRVAGPQRRPV-PGSIAT-MVSKGEELFTGVVPILV
ELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRA
EVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQ
SALSKDPNEKRDHMVLLEFVTAAGITLGMDELYK

The above-listed amino acid sequence contains the amino acid sequences of a truncated Herpes thymidine kinase and enhanced green fluorescent protein (EGFP) linked by six amino acid linker sequence (underlined).

A TK-EGFP fusion polypeptide is encoded by the following nucleotide sequence:

(SEQ ID NO: 30)
ATGgcttcgtaccectgccatcaacacgcgtagcgttcgaccaggctg
cgcgttctcgcggccataacaaccgacgtacggcgttgcgccctcgcc
ggcaacaaaaagccacggaagtccgcctggagcagaaaatgcccacgc
tactgcgggtttatatagacggtccccacgggatggggaaaaccacca
ccacgcaactgctggtggccagggttcgcgcgacgatatcgtctacgt
acccgagccgatgacttactggcgggtgttgggggcttccgagacaat
cgcgaacatctacaccacacaacaccgcctcgaccagggtgagatatc
ggccggggacgcggcggtggtaatgacaagcgcccagataacaatggg
catgccttatgccgtgaccgacgccgttctggctcctcatatcggggg
ggaggctgggagctcacatgccccgcccccggccctcaccctcatctt
cgaccgccatcccatcgccgccctcctgtgctacccggccgcgcgata
ccttatgggcagcatgaccccccaggccgtgctggcgttcgtggccct
catcccgccgaccttgcccggcacaaacatcgtgttgggggccatccg
gaggacagacacatcgaccgcctggccaaacgccagcgccccggcgag
cggcttgacctggctatgctggccgcgtcgccgcgtttatgggctgat
gccaatacggtgcggtatctgcagggcggcgggtcgtggcgggaggat
tggggacagctttcgggggcggccgtgccgccccagggtgccgagccc
cagagcaacgcgggcccacgaccccatatcggggacacgttatttacc
ctgtttcgggcccccgagttgctggcccccaacggcgacctgtaCCTG
GTTCTATTGCTACTatggtgagcaagggcgaggagctgttcaccgggg
tggtgcccatcctggtcgagctggacggcgacgtaaacggccacaagt
tcagcgtgtccggcgagggcgagggcgatgccacctacggcaagctga
ccctgaagttcatctgcaccaccggcaagctgcccgtgccctggccca
ccctcgtgaccaccctgacctacggcgtgcagtgcttcagccgctacc
ccgaccacatgaagcagcacgacttcttcaagtccgccatgcccgaag
gctacgtccaggagcgcaccatcttcttcaaggacgacggcaactaca
agacccgcgccgaggtgaagttcgagggcgacaccctggtgaaccgca
tcgagctgaagggcatcgacttcaaggaggacggcaacatcctggggc
acaagctggagtacaactacaacagccacaacgtctatatcatggccg
acaagcagaagaacggcatcaaggtgaacttcaagatccgccacaaca
tcgaggacggcagcgtgcagctcgccgaccactaccagcagaacaccc
ccatcggcgacggccccgtgctgctgcccgacaaccactacctgagca
cccagtccgccctgagcaaagaccccaacgagaagcgcgatcacatgg
tcctgctggagttcgtgaccgccgccgggatcactctcggcatggacg
agctgtacaagtaa

### Example 3

### iPSC Derivation using Viral Vector Conditionally Lethal Protein-Fluoresent Timer Fusion Protein Gene Selection

The time course of epigenetic silencing of retroviral transgene expression in induced cells is relatively long (at least about two weeks) and may be quite variable between clones and different cell types. Thus, the optimal timing of negative selection for putative iPSC clones may also be quite variable. Example 2 describes the use of a bifunctional TK-EGFP fusion protein to allow both monitoring of viral transgene expression over time by epifluorescence and suicide gene negative selection by incubation with the TK-activated prodrug ganciclovir. Over time, as viral transgene expression is silenced, the level of EGFP fluorescence will wane. However, the absolute level of EGFP fluorescence from clone to clone is expected to be variable, and therefore for clones showing very low EGFP fluorescence initially, it may be difficult to distinguish changes in absolute EGFP fluorescence over time. The use of fluorescent timer protein (see Terskikh et al (2000), Science, 290(5496):1585-1588, and U.S. Pat. No. 7,217,789) as the fluorescent protein in a TK-fluorescent timer fusion protein allows kinetic monitoring of viral transgene expression based on the ratio of “mature” timer (red) to immature timer (green), a readout which is independent of the absolute concentration of the timer protein. It is expected that as viral transgene expression is silenced and less newly translated (immature) timer is present in the induced cells, the ratio of red to green fluorescence will increase providing a readout of viral transgene expression/silencing regardless of the concentration of the fluorescent timer protein at the beginning of the induction period. The amino acid sequence of an exemplary TK-TIMER fusion polypeptide is shown below:

TK-TIMER:

(SEQ ID NO: 31)
MASYPCHQHASAFDQAARSRGHNNRRTALRPRRQQKATEVRLEQKMP
TLLRVYIDGPHGMGKTTTTQLLVALGSRDDIVYVPEPMTYWRVLGAS
ETIANIYTTQHRLDQGEISAGDAAVVMTSAQITMGMPYAVTDAVLAP
HIGGEAGSSHAPPPALTLIFDRHPIAALLCYPAARYLMGSMTPQAVL
AFVALIPPTLPGTNIVLGALPEDRHIDRLAKRQRPGERLDLAMLAAS
PRLWAACQYGAVSAGRRVVAGGLGTAFGGGRAAPGCRAPEQRGPTTP
YRGHVIYPVSGPRVAGPQRRPV-PGSIAT-MVRSSKNVIKEFMRFKV
RMEGTVNGHEFEIEGEGEGRPYEGHNTVKLKVTKGGPLPFAWDILSP
QFQYGSKVYVKHPADIPDYKKLSFPEGFKWERVMNFEDGGVATVTQD
SSLQDGCFIYKVKFIGVNFPSDGPVMQKKTMGWEASTERLYPRDGVL
KGEIHKALKLKDGGHYLVEFKSIYMAKKPVQLPGYYYVDTKLDITSH
NEDYTIVEQYERTEGRHHLFL

A TK-TIMER fusion polypeptide is encoded by the following nucleotide sequence:

(SEQ ID NO: 32)
ATGgatcgtaccectgccatcaacacgcgtctgcgttcgaccaggc
tgcgcgttctcgcggccataacaaccgacgtacggcgttgcgccct
cgccggcaacaaaaagccacggaagtccgcctggagcagaaaatgc
ccacgctactgcgggtttatatagacggtccccacgggatggggaa
aaccaccaccacgcaactgctggtggccctgggttcgcgcgacgat
atcgtctacgtacccgagccgatgacttactggcgggtgttggggg
cttccgagacaatcgcgaacatctacaccacacaacaccgcctcga
ccagggtgagatatcggccggggacgcggcggtggtaatgacaagc
gcccagataacaatgggcatgccttatgccgtgaccgacgccgttc
tggctcctcatatcgggggggaggctgggagctcacatgccccgcc
cccggccctcaccctcatcttcgaccgccatcccatcgccgccctc
ctgtgctacccggccgcgcgataccttatgggcagcatgacccccc
aggccgtgctggcgttcgtggccctcatcccgccgaccttgcccgg
cacaaacatcgtgttgggggccatccggaggacagacacatcgacc
gcctggccaaacgccagcgccccggcgagcggcttgacctggctat
gctggccgcgtcgccgcgtttatgggctgatgccaatacggtgcgg
tatctgcagggcggcgggtcgtggcgggaggattggggacagcttt
cgggggcggccgtgccgccccagggtgccgagccccagagcaacgc
gggcccacgaccccatatcggggacacgttatttaccctgtttcgg
gcccccgagttgctggcccccaacggcgacctgtaCCTGGTTCTAT
TGCTACTatggtgcgctcctccaagaacgtcatcaaggagttcatg
cgcttcaaggtgcgcatggagggcaccgtgaacggccacgagttcg
agatcgagggcgagggcgagggccgcccctacgagggccacaacac
cgtgaagctgaaggtgaccaagggcggccccctgcccttcgcctgg
gacatcctgtccccccagttccagtacggctccaaggtgtacgtga
agcaccccgccgacatccccgactacaagaagctgtccttccccga
gggcttcaagtgggagcgcgtgatgaacttcgaggacggcggcgtg
gcgaccgtgacccaggactcctccctgcaggacggctgcttcatct
acaaggtgaagttcatcggcgtgaacttcccctccgacggccccgt
gatgcagaagaagaccatgggctgggaggcctccaccgagcgcctg
tacccccgcgacggcgtgctgaagggcgagatccacaaggccctga
agctgaaggacggcggccactacctggtggagttcaagtccatcta
catggccaagaagcccgtgcagctgcccggctactactacgtggac
accaagctggacatcacctcccacaacgaggactacaccatcgtgg
agcagtacgagcgcaccgagggccgccaccacctgttcctgtag

Induction is performed as described in example 1, but transduction is carried out with:
(A): three MoMLV retroviruses containing expression cassettes for human Klf4-IRES-tk-timer, human Sox2-IRES-tk-timer, and human Oct 4-IRES-tk-timer, respectively; or.
(B): four MoMLV retroviruses containing expression cassettes for human Klf4-IRES-tk-timer, human Sox2-IRES-tk-timer, human Oct 4-IRES-tk-timer, and human c-Myc-IRES-tk-timer.
   The ratio of red fluorescence to green fluorescence is assessed every 4-5 days, and, at the earliest, ganciclovir selection is initiated when essentially only red fluorescence is observed in the cells (i.e., no new expression of TK-TIMER is occurring), or selection begins after even red fluorescence is no longer observed in the putative iPSC clones. After pluripotency validation of iPSC clones that survive negative selection, changes in the red/green emission over time are compared in clones that survived ganciclovir negative selection versus those that did not survive and those that held a constant ratio of red/green fluorescence. This is likely to reveal a kinetic signature that will allow early identification of putative iPSCs most likely to reprogram successfully based on the time course of changes in the red to green emission ratio.

### Example 4

### iPSC Derivation using Combined Positive and Negative Selection

As reprogramming, to date, is an inefficient process, it is useful to improve the efficiency of the various steps required for successful induction of pluripotent iPSCs. In one approach to induction, Oct 4, Sox2, Klf4, and optionally c-Myc are transduced using separate MoMLV retroviruses. On the one hand successful induction/reprogramming of a cell requires the introduction of at least Oct 4, Sox2, and Klf4 transgenes, but on the other hand although retroviral transduction is very efficient, this process becomes increasingly less efficient with each additional retrovirus. Indeed, both in theory and in practice, only a fraction of the total cell population incubated with the above-mentioned retroviral cocktails cells are successfully transduced with the three or more induction factor transgenes. Thus, it is useful to select at the beginning of the induction process only those cells that have been transduced with and express three induction factor transgenes (i.e., Oct 4, Klf4, and Sox 2) or four induction factor transgenes (i.e., Oct 4, Klf4, Sox 2, and c-Myc) depending on the induction protocol used. The use of retroviruses containing bicistronic expression vectors for co-expression of an induction factor and a distinct selection marker for positive selection allows an initial enrichment for colonies that express all of the necessary induction factors and may therefore become reprogrammed. Combined positive and negative selection of iPS cells is illustrated schematically in FIG. 5.

Human fibroblasts are induced as described in Example 1, but the expression cassettes and corresponding viral packaging constructs (where indicated) for the separate retroviruses are as follows (see also FIGS. 6, 7 and vector sequence appendix).

1. Oct 4-IRES-Hygromycin Phosphotransferase (pMXs-Oct4-IRES-Hygro)

2. Sox2-IRES-Puromycin Acetyltransferase (pMXs-Sox2-IRES-Puro)

3. Klf4-IRES-Neomycin Phosphotransferase (pMXs-Kl4-IRES-neo)

4. c-Myc-IRES-TK (pMXs-c-Myc-IRES-TK), c-Myc-IRES-TK-EGFP, or c-Myc-IRES-TK-TIMER

Approximately 48 hours after addition of viral transduction, cells are cultured in the presence of hygromycin (200 μg/ml); puromycin (2 μg/ml), and neomycin (400 μg/ml) for approximately one week to ensure elimination of cells that do not express at least the Oct 4, Sox2, and Klf4 transgenes. Where a c-Myc-IRES TK-fluorescent protein is used, fluorescent reporter protein fluorescence is assessed in the putative iPSC colonies that survive triple drug positive selection and monitored weekly until fluorescent protein signal is no longer observed in the cells, or after approximately one month when no fluorescent reporter virus is utilized. Subsequently, putative iPSC clones that survive negative selection in ganciclovir are tested for pluripotency as described in Example 1.

### APPENDIX

### Viral Packaging Vector Construct Sequences

pMXs-Oct4-IRES-TK

| (SEQ ID NO: 33) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | cccagtgtgg tggtacggga attcccttcg caagccctca tttcaccagg cccccggctt |
| 1981 | ggggcgcctt ccttccccat ggcgggacac ctggcttcgg atttcgcctt ctcgccccct |
| 2041 | ccaggtggtg gaggtgatgg gccagggggg ccggagccgg gctgggttga tcctcggacc |
| 2101 | tggctaagct tccaaggccc tcctggaggg ccaggaatcg ggccgggggt tgggccaggc |
| 2161 | tctgaggtgt gggggattcc cccatgcccc ccgccgtatg agttctgtgg ggggatggcg |
| 2221 | tactgtgggc cccaggttgg agtggggcta gtgccccaag gcggcttgga gacctctcag |
| 2281 | cctgagggcg aagcaggagt cggggtggag agcaactccg atggggcctc cccggagccc |
| 2341 | tgcaccgtca cccctggtgc cgtgaagctg gagaaggaga agctggagca aaacccggag |
| 2401 | gagtcccagg acatcaaagc tctgcagaaa gaactcgagc aatttgccaa gctcctgaag |
| 2461 | cagaagagga tcaccctggg atatacacag gccgatgtgg ggctcaccct gggggttcta |
| 2521 | tttgggaagg tattcagcca aacgaccatc tgccgctttg aggctctgca gcttagcttc |
| 2581 | aagaacatgt gtaagctgcg gcccttgctg cagaagtggg tggaggaagc tgacaacaat |
| 2641 | gaaaatcttc aggagatatg caaagcagaa accctcgtgc aggcccgaaa gagaaagcga |
| 2701 | accagtatcg agaaccgagt gagaggcaac ctggagaatt tgttcctgca gtgcccgaaa |
| 2761 | cccacactgc agcagatcag ccacatcgcc cagcagcttg ggctcgagaa ggatgtggtc |
| 2821 | cgagtgtggt tctgtaaccg gcgccagaag ggcaagcgat caagcagcga ctatgcacaa |
| 2881 | cgagaggatt ttgaggctgc tgggtctcct ttctcagggg gaccagtgtc ctttcctctg |
| 2941 | gccccagggc cccattttgg taccccaggc tatgggagcc ctcacttcac tgcactgtac |
| 3001 | tcctcggtcc ctttccctga gggggaagcc tttccccctg tctccgtcac cactctgggc |
| 3061 | tctcccatgc attcaaactg aggtgcctgc ccttctagga atgggggaca gggggagggg |
| 3121 | aggagctagg gaagaattcg cggcaattcc tgcaggcctc gagggccggc gcgccgcggc |
| 3181 | cgcgactcta gaatttcgac ctcgacatta attccggtta ttttccacca tattgccgtc |
| 3241 | ttttggcaat gtgagggccc ggaaacctgg ccctgtcttc ttgacgagca ttcctagggg |
| 3301 | tctttcccct ctcgccaaag gaatgcaagg tctgttgaat gtcgtgaagg aagcagttcc |
| 3361 | tctggaagct tcttgaagac aaacaacgtc tgtagcgacc ctttgcaggc agcggaaccc |
| 3421 | cccacctggc gacaggtgcc tctgcggcca aaagccacgt gtataagata cacctgcaaa |
| 3481 | ggcggcacaa ccccagtgcc acgttgtgag ttggatagtt gtggaaagag tcaaatggct |
| 3541 | ctcctcaagc gtattcaaca aggggctgaa ggatgcccag aaggtacccc attgtatggg |
| 3601 | atctgatctg gggcctcggt gcacatgctt tacatgtgtt tagtcgaggt taaaaaacgt |
| 3661 | ctaggccccc cgaaccacgg ggacgtggtt ttcctttgaa aaacacgatg ataataccat |
| 3721 | ggcttcgtac ccctgccatc aacacgcgtc tgcgttcgac caggctgcgc gttctcgcgg |
| 3781 | ccataacaac cgacgtacgg cgttgcgccc tcgccggcaa caaaaagcca cggaagtccg |
| 3841 | cctggagcag aaaatgccca cgctactgcg ggtttatata gacggtcccc acgggatggg |
| 3901 | gaaaaccacc accacgcaac tgctggtggc cctgggttcg cgcgacgata tcgtctacgt |
| 3961 | acccgagccg atgacttact ggcgggtgtt gggggcttcc gagacaatcg cgaacatcta |
| 4021 | caccacacaa caccgcctcg accagggtga gatatcggcc ggggacgcgg cggtggtaat |
| 4081 | gacaagcgcc cagataacaa tgggcatgcc ttatgccgtg accgacgccg ttctggctcc |
| 4141 | tcatatcggg ggggaggctg ggagctcaca tgccccgccc ccggccctca ccctcatctt |
| 4201 | cgaccgccat cccatcgccg ccctcctgtg ctacccggcc gcgcgatacc ttatgggcag |
| 4261 | catgaccccc caggccgtgc tggcgttcgt ggccctcatc ccgccgacct tgcccggcac |
| 4321 | aaacatcgtg ttgggggccc ttccggagga cagacacatc gaccgcctgg ccaaacgcca |
| 4381 | gcgccccggc gagcggcttg acctggctat gctggccgcg tcgccgcgtt tatgggctgc |
| 4441 | ttgccaatac ggtgcggtat ctgcagggcg gcgggtcgtg gcgggaggat tggggacagc |
| 4501 | tttcgggggc ggccgtgccg ccccagggtg ccgagcccca gagcaacgcg ggcccacgac |
| 4561 | cccatatcgg ggacacgtta tttaccctgt ttcgggcccc cgagttgctg gcccccaacg |
| 4621 | gcgacctgta taacgtgttt gcctgggctt tggctcgacg gtacctttaa gaccaatgac |
| 4681 | ttacaaggca gctgtagatc aattcgatat caagcttatc gataatcaac ctctggatta |
| 4741 | caaaatttgt gaaagattga ctggtattct taactatgtt gctcctttta cgctatgtgg |
| 4801 | atacgctgct ttaatgcctt tgtatcatgc tattgcttcc cgtatggctt tcattttctc |
| 4861 | ctccttgtat aaatcctggt tgctgtctct ttatgaggag ttgtggcccg ttgtcaggca |
| 4921 | acgtggcgtg gtgtgcactg tgtttgctga cgcaaccccc actggttggg gcattgccac |
| 4981 | cacctgtcag ctcctttccg ggactttcgc tttccccctc cctattgcca cggcggaact |
| 5041 | catcgccgcc tgccttgccc gctgctggac aggggctcgg ctgttgggca ctgacaattc |
| 5101 | cgtggtgttg tcggggaaat catcgtcctt tccttggctg ctcgcctgtg ttgccacctg |
| 5161 | gattctgcgc gggacgtcct tctgctacgt cccttcggcc ctcaatccag cggaccttcc |
| 5221 | ttcccgcggc ctgctgccgg ctctgcggcc tcttccgcgt cttcgccttc gccctcagac |
| 5281 | gagtcggatc tccctttggg ccgcctcccc gcatcgatac cgtcgacgat aaaataaaag |
| 5341 | attttattta gtctccagaa aaagggggga atgaaagacc ccacctgtag gtttggcaag |
| 5401 | ctagcttaag taacgccatt ttgcaaggca tggaaaaata cataactgag aatagagaag |
| 5461 | ttcagatcaa ggtcaggaac agatggaaca gctgaatatg ggccaaacag gatatctgtg |
| 5521 | gtaagcagtt cctgccccgg ctcagggcca agaacagatg gaacagctga atatgggcca |
| 5581 | aacaggatat ctgtggtaag cagttcctgc cccggctcag ggccaagaac agatggtccc |
| 5641 | cagatgcggt ccagccctca gcagtttcta gagaaccatc agatgtttcc agggtgcccc |
| 5701 | aaggacctga aatgaccctg tgccttattt gaactaacca atcagttcgc ttctcgcttc |
| 5761 | tgttcgcgcg cttctgctcc ccgagctcaa taaaagagcc cacaacccct cactcggggc |
| 5821 | gccagtcctc cgattgactg agtcgcccgg gtacccgtgt atccaataaa ccctcttgca |
| 5881 | gttgcatccg acttgtggtc tcgctgttcc ttgggagggt ctcctctgag tgattgacta |
| 5941 | cccgtcagcg ggggtctttc acatgcagca tgtatcaaaa ttaatttggt tttttttctt |
| 6001 | aagtatttac attaaatggc catagttgca ttaatgaatc ggccaacgcg cggggagagg |
| 6061 | cggtttgcgt attgggcgct cttccgcttc ctcgctcact gactcgctgc gctcggtcgt |
| 6121 | tcggctgcgg cgagcggtat cagctcactc aaaggcggta atacggttat ccacagaatc |
| 6181 | aggggataac gcaggaaaga acatgtgagc aaaaggccag caaaaggcca ggaaccgtaa |
| 6241 | aaaggccgcg ttgctggcgt ttttccatag gctccgcccc cctgacgagc atcacaaaaa |
| 6301 | tcgacgctca agtcagaggt ggcgaaaccc gacaggacta taaagatacc aggcgtttcc |
| 6361 | ccctggaagc tccctcgtgc gctctcctgt tccgaccctg ccgcttaccg gatacctgtc |
| 6421 | cgcctttctc ccttcgggaa gcgtggcgct ttctcatagc tcacgctgta ggtatctcag |
| 6481 | ttcggtgtag gtcgttcgct ccaagctggg ctgtgtgcac gaaccccccg ttcagcccga |
| 6541 | ccgctgcgcc ttatccggta actatcgtct tgagtccaac ccggtaagac acgacttatc |
| 6601 | gccactggca gcagccactg gtaacaggat tagcagagcg aggtatgtag gcggtgctac |
| 6661 | agagttcttg aagtggtggc ctaactacgg ctacactaga agaacagtat ttggtatctg |
| 6721 | cgctctgctg aagccagtta ccttcggaaa aagagttggt agctcttgat ccggcaaaca |
| 6781 | aaccaccgct ggtagcggtg gtttttttgt ttgcaagcag cagattacgc gcagaaaaaa |
| 6841 | aggatctcaa gaagatcctt tgatcttttc tacggggtct gacgctcagt ggaacgaaaa |
| 6901 | ctcacgttaa gggattttgg tcatgagatt atcaaaaagg atcttcacct agatcctttt |
| 6961 | gcggccggcc gcaaatcaat ctaaagtata tatgagtaaa cttggtctga cagttaccaa |
| 7021 | tgcttaatca gtgaggcacc tatctcagcg atctgtctat ttcgttcatc catagttgcc |
| 7081 | tgactccccg tcgtgtagat aactacgata cgggagggct taccatctgg ccccagtgct |
| 7141 | gcaatgatac cgcgagaccc acgctcaccg gctccagatt tatcagcaat aaaccagcca |
| 7201 | gccggaaggg ccgagcgcag aagtggtcct gcaactttat ccgcctccat ccagtctatt |
| 7261 | aattgttgcc gggaagctag agtaagtagt tcgccagtta atagtttgcg caacgttgtt |
| 7321 | gccattgcta caggcatcgt ggtgtcacgc tcgtcgtttg gtatggcttc attcagctcc |
| 7381 | ggttcccaac gatcaaggcg agttacatga tcccccatgt tgtgcaaaaa agcggttagc |
| 7441 | tccttcggtc ctccgatcgt tgtcagaagt aagttggccg cagtgttatc actcatggtt |
| 7501 | atggcagcac tgcataattc tcttactgtc atgccatccg taagatgctt ttctgtgact |
| 7561 | ggtgagtact caaccaagtc attctgagaa tagtgtatgc ggcgaccgag ttgctcttgc |
| 7621 | ccggcgtcaa tacgggataa taccgcgcca catagcagaa ctttaaaagt gctcatcatt |
| 7681 | ggaaaacgtt cttcggggcg aaaactctca aggatcttac cgctgttgag atccagttcg |
| 7741 | atgtaaccca ctcgtgcacc caactgatct tcagcatctt ttactttcac cagcgtttct |
| 7801 | gggtgagcaa aaacaggaag gcaaaatgcc gcaaaaaagg gaataagggc gacacggaaa |
| 7861 | tgttgaatac tcatactctt cctttttcaa tattattgaa gcatttatca gggttattgt |
| 7921 | ctcatgagcg gatacatatt tgaatgtatt tagaaaaata aacaaatagg ggttccgcgc |
| 7981 | acatttc |

pMXs-Sox2-IRES-TK

| (SEQ ID NO: 34) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | cccagtgtgg tggtacggga attccccggg ccccccaaag tcccggccgg gccgagggtc |
| 1981 | ggcggccgcc ggcgggccgg gcccgcgcac agcgcccgca tgtacaacat gatggagacg |
| 2041 | gagctgaagc cgccgggccc gcagcaaact tcggggggcg gcggcggcaa ctccaccgcg |
| 2101 | gcggcggccg gcggcaacca gaaaaacagc ccggaccgcg tcaagcggcc catgaatgcc |
| 2161 | ttcatggtgt ggtcccgcgg gcagcggcgc aagatggccc aggagaaccc caagatgcac |
| 2221 | aactcggaga tcagcaagcg cctgggcgcc gagtggaaac ttttgtcgga gacggagaag |
| 2281 | cggccgttca tcgacgaggc taagcggctg cgagcgctgc acatgaagga gcacccggat |
| 2341 | tataaatacc ggccccggcg gaaaaccaag acgctcatga agaaggataa gtacacgctg |
| 2401 | cccggcgggc tgctggcccc cggcggcaat agcatggcga gcggggtcgg ggtgggcgcc |
| 2461 | ggcctgggcg cgggcgtgaa ccagcgcatg gacagttacg cgcacatgaa cggctggagc |
| 2521 | aacggcagct acagcatgat gcaggaccag ctgggctacc cgcagcaccc gggcctcaat |
| 2581 | gcgcacggcg cagcgcagat gcagcccatg caccgctacg acgtgagcgc cctgcagtac |
| 2641 | aactccatga ccagctcgca gacctacatg aacggctcgc ccacctacag catgtcctac |
| 2701 | tcgcagcagg gcacccctgg catggctctt ggctccatgg gttcggtggt caagtccgag |
| 2761 | gccagctcca gcccccctgt ggttacctct tcctcccact ccagggcgcc ctgccaggcc |
| 2821 | ggggacctcc gggacatgat cagcatgtat ctccccggcg ccgaggtgcc ggaacccgcc |
| 2881 | gcccccagca gacttcacat gtcccagcac taccagagcg gcccggtgcc cggcacggcc |
| 2941 | attaacggca cactgcccct ctcacacatg tgagggccgg acagcgaact ggagggggga |
| 3001 | gaaattttca aagaaaaacg agggaaatgg gaggggtgca aaagaggaga gtaagaaaca |
| 3061 | gcatggagaa aacccggtac gctcaaaaaa aaaaaaaaaa aaaaaaaaaa aaaaaactcg |
| 3121 | agggccggcg cgccgcggcc gcgactctag aatttcgacc tcgacattaa ttccggttat |
| 3181 | tttccaccat attgccgtct tttggcaatg tgagggcccg gaaacctggc cctgtcttct |
| 3241 | tgacgagcat tcctaggggt ctttcccctc tcgccaaagg aatgcaaggt ctgttgaatg |
| 3301 | tcgtgaagga agcagttcct ctggaagctt cttgaagaca aacaacgtct gtagcgaccc |
| 3361 | tttgcaggca gcggaacccc ccacctggcg acaggtgcct ctgcggccaa aagccacgtg |
| 3421 | tataagatac acctgcaaag gcggcacaac cccagtgcca cgttgtgagt tggatagttg |
| 3481 | tggaaagagt caaatggctc tcctcaagcg tattcaacaa ggggctgaag gatgcccaga |
| 3541 | aggtacccca ttgtatggga tctgatctgg ggcctcggtg cacatgcttt acatgtgttt |
| 3601 | agtcgaggtt aaaaaacgtc taggcccccc gaaccacggg gacgtggttt tcctttgaaa |
| 3661 | aacacgatga taataccatg gcttcgtacc cctgccatca acacgcgtct gcgttcgacc |
| 3721 | aggctgcgcg ttctcgcggc cataacaacc gacgtacggc gttgcgccct cgccggcaac |
| 3781 | aaaaagccac ggaagtccgc ctggagcaga aaatgcccac gctactgcgg gtttatatag |
| 3841 | acggtcccca cgggatgggg aaaaccacca ccacgcaact gctggtggcc ctgggttcgc |
| 3901 | gcgacgatat cgtctacgta cccgagccga tgacttactg gcgggtgttg ggggcttccg |
| 3961 | agacaatcgc gaacatctac accacacaac accgcctcga ccagggtgag atatcggccg |
| 4021 | gggacgcggc ggtggtaatg acaagcgccc agataacaat gggcatgcct tatgccgtga |
| 4081 | ccgacgccgt tctggctcct catatcgggg gggaggctgg gagctcacat gccccgcccc |
| 4141 | cggccctcac cctcatcttc gaccgccatc ccatcgccgc cctcctgtgc tacccggccg |
| 4201 | cgcgatacct tatgggcagc atgacccccc aggccgtgct ggcgttcgtg gccctcatcc |
| 4261 | cgccgacctt gcccggcaca aacatcgtgt tgggggccct tccggaggac agacacatcg |
| 4321 | accgcctggc caaacgccag cgccccggcg agcggcttga cctggctatg ctggccgcgt |
| 4381 | cgccgcgttt atgggctgct tgccaatacg gtgcggtatc tgcagggcgg cgggtcgtgg |
| 4441 | cgggaggatt ggggacagct ttcgggggcg gccgtgccgc cccagggtgc cgagccccag |
| 4501 | agcaacgcgg gcccacgacc ccatatcggg gacacgttat ttaccctgtt tcgggccccc |
| 4561 | gagttgctgg cccccaacgg cgacctgtat aacgtgtttg cctgggcttt ggctcgacgg |
| 4621 | tacctttaag accaatgact tacaaggcag ctgtagatca attcgatatc aagcttatcg |
| 4681 | ataatcaacc tctggattac aaaatttgtg aaagattgac tggtattctt aactatgttg |
| 4741 | ctccttttac gctatgtgga tacgctgctt taatgccttt gtatcatgct attgcttccc |
| 4801 | gtatggcttt cattttctcc tccttgtata aatcctggtt gctgtctctt tatgaggagt |
| 4861 | tgtggcccgt tgtcaggcaa cgtggcgtgg tgtgcactgt gtttgctgac gcaaccccca |
| 4921 | ctggttgggg cattgccacc acctgtcagc tcctttccgg gactttcgct ttccccctcc |
| 4981 | ctattgccac ggcggaactc atcgccgcct gccttgcccg ctgctggaca ggggctcggc |
| 5041 | tgttgggcac tgacaattcc gtggtgttgt cggggaaatc atcgtccttt ccttggctgc |
| 5101 | tcgcctgtgt tgccacctgg attctgcgcg ggacgtcctt ctgctacgtc ccttcggccc |
| 5161 | tcaatccagc ggaccttcct tcccgcggcc tgctgccggc tctgcggcct cttccgcgtc |
| 5221 | ttcgccttcg ccctcagacg agtcggatct ccctttgggc cgcctccccg catcgatacc |
| 5281 | gtcgacgata aaataaaaga ttttatttag tctccagaaa aaggggggaa tgaaagaccc |
| 5341 | cacctgtagg tttggcaagc tagcttaagt aacgccattt tgcaaggcat ggaaaaatac |
| 5401 | ataactgaga atagagaagt tcagatcaag gtcaggaaca gatggaacag ctgaatatgg |
| 5461 | gccaaacagg atatctgtgg taagcagttc ctgccccggc tcagggccaa gaacagatgg |
| 5521 | aacagctgaa tatgggccaa acaggatatc tgtggtaagc agttcctgcc ccggctcagg |
| 5581 | gccaagaaca gatggtcccc agatgcggtc cagccctcag cagtttctag agaaccatca |
| 5641 | gatgtttcca gggtgcccca aggacctgaa atgaccctgt gccttatttg aactaaccaa |
| 5701 | tcagttcgct tctcgcttct gttcgcgcgc ttctgctccc cgagctcaat aaaagagccc |
| 5761 | acaacccctc actcggggcg ccagtcctcc gattgactga gtcgcccggg tacccgtgta |
| 5821 | tccaataaac cctcttgcag ttgcatccga cttgtggtct cgctgttcct tgggagggtc |
| 5881 | tcctctgagt gattgactac ccgtcagcgg gggtctttca catgcagcat gtatcaaaat |
| 5941 | taatttggtt ttttttctta agtatttaca ttaaatggcc atagttgcat taatgaatcg |
| 6001 | gccaacgcgc ggggagaggc ggtttgcgta ttgggcgctc ttccgcttcc tcgctcactg |
| 6061 | actcgctgcg ctcggtcgtt cggctgcggc gagcggtatc agctcactca aaggcggtaa |
| 6121 | tacggttatc cacagaatca ggggataacg caggaaagaa catgtgagca aaaggccagc |
| 6181 | aaaaggccag gaaccgtaaa aaggccgcgt tgctggcgtt tttccatagg ctccgccccc |
| 6241 | ctgacgagca tcacaaaaat cgacgctcaa gtcagaggtg gcgaaacccg acaggactat |
| 6301 | aaagatacca ggcgtttccc cctggaagct ccctcgtgcg ctctcctgtt ccgaccctgc |
| 6361 | cgcttaccgg atacctgtcc gcctttctcc cttcgggaag cgtggcgctt tctcatagct |
| 6421 | cacgctgtag gtatctcagt tcggtgtagg tcgttcgctc caagctgggc tgtgtgcacg |
| 6481 | aaccccccgt tcagcccgac cgctgcgcct tatccggtaa ctatcgtctt gagtccaacc |
| 6541 | cggtaagaca cgacttatcg ccactggcag cagccactgg taacaggatt agcagagcga |
| 6601 | ggtatgtagg cggtgctaca gagttcttga agtggtggcc taactacggc tacactagaa |
| 6661 | gaacagtatt tggtatctgc gctctgctga agccagttac cttcggaaaa agagttggta |
| 6721 | gctcttgatc cggcaaacaa accaccgctg gtagcggtgg tttttttgtt tgcaagcagc |
| 6781 | agattacgcg cagaaaaaaa ggatctcaag aagatccttt gatcttttct acggggtctg |
| 6841 | acgctcagtg gaacgaaaac tcacgttaag ggattttggt catgagatta tcaaaaagga |
| 6901 | tcttcaccta gatccttttg cggccggccg caaatcaatc taaagtatat atgagtaaac |
| 6961 | ttggtctgac agttaccaat gcttaatcag tgaggcacct atctcagcga tctgtctatt |
| 7021 | tcgttcatcc atagttgcct gactccccgt cgtgtagata actacgatac gggagggctt |
| 7081 | accatctggc cccagtgctg caatgatacc gcgagaccca cgctcaccgg ctccagattt |
| 7141 | atcagcaata aaccagccag ccggaagggc cgagcgcaga agtggtcctg caactttatc |
| 7201 | cgcctccatc cagtctatta attgttgccg ggaagctaga gtaagtagtt cgccagttaa |
| 7261 | tagtttgcgc aacgttgttg ccattgctac aggcatcgtg gtgtcacgct cgtcgtttgg |
| 7321 | tatggcttca ttcagctccg gttcccaacg atcaaggcga gttacatgat cccccatgtt |
| 7381 | gtgcaaaaaa gcggttagct ccttcggtcc tccgatcgtt gtcagaagta agttggccgc |
| 7441 | agtgttatca ctcatggtta tggcagcact gcataattct cttactgtca tgccatccgt |
| 7501 | aagatgcttt tctgtgactg gtgagtactc aaccaagtca ttctgagaat agtgtatgcg |
| 7561 | gcgaccgagt tgctcttgcc cggcgtcaat acgggataat accgcgccac atagcagaac |
| 7621 | tttaaaagtg ctcatcattg gaaaacgttc ttcggggcga aaactctcaa ggatcttacc |
| 7681 | gctgttgaga tccagttcga tgtaacccac tcgtgcaccc aactgatctt cagcatcttt |
| 7741 | tactttcacc agcgtttctg ggtgagcaaa aacaggaagg caaaatgccg caaaaaaggg |
| 7801 | aataagggcg acacggaaat gttgaatact catactcttc ctttttcaat attattgaag |
| 7861 | catttatcag ggttattgtc tcatgagcgg atacatattt gaatgtattt agaaaaataa |
| 7921 | acaaataggg gttccgcgca catttc |

pMXs-Klf4-IRES-TK

| (SEQ ID NO: 35) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | cccagtgtgg tggtacggga attctcgagg cgaccgcgac agtggtgggg gacgctgctg |
| 1981 | agtggaagag agcgcagccc ggccaccgga cctacttact cgccttgctg attgtctatt |
| 2041 | tttgcgttta caacttttct aagaactttt gtatacaaag gaacttttta aaaaagacgc |
| 2101 | ttccaagtta tatttaatcc aaagaagaag gatctcggcc aatttggggt tttgggtttt |
| 2161 | ggcttcgttt cttctcttcg ttgactttgg ggttcaggtg ccccagctgc ttcgggctgc |
| 2221 | cgaggacctt ctgggccccc acattaatga ggcagccacc tggcgagtct gacatggctg |
| 2281 | tcagcgacgc gctgctccca tctttctcca cgttcgcgtc tggcccggcg ggaagggaga |
| 2341 | agacactgcg tcaagcaggt gccccgaata accgctggcg ggaggagctc tcccacatga |
| 2401 | agcgacttcc cccagtgctt cccggccgcc cctatgacct ggcggcggcg accgtggcca |
| 2461 | cagacctgga gagcggcgga gccggtgcgg cttgcggcgg tagcaacctg gcgcccctac |
| 2521 | ctcggagaga gaccgaggag ttcaacgatc tcctggacct ggactttatt ctctccaatt |
| 2581 | cgctgaccca tcctccggag tcagtggccg ccaccgtgtc ctcgtcagcg tcagcctcct |
| 2641 | cttcgtcgtc gccgtcgagc agcggccctg ccagcgcgcc ctccacctgc agcttcacct |
| 2701 | atccgatccg ggccgggaac gacccgggcg tggcgccggg cggcacgggc ggaggcctcc |
| 2761 | tctatggcag ggagtccgct ccccctccga cggctccctt caacctggcg gacatcaacg |
| 2821 | acgtgagccc ctcgggcggc ttcgtggccg agctcctgcg gccagaattg gacccggtgt |
| 2881 | acattccgcc gcagcagccg cagccgccag gtggcgggct gatgggcaag ttcgtgctga |
| 2941 | aggcgtcgct gagcgcccct ggcagcgagt acggcagccc gtcggtcatc agcgtcagca |
| 3001 | aaggcagccc tgacggcagc cacccggtgg tggtggcgcc ctacaacggc gggccgccgc |
| 3061 | gcacgtgccc caagatcaag caggaggcgg tctcttcgtg cacccacttg ggcgctggac |
| 3121 | cccctctcag caatggccac cggccggctg cacacgactt ccccctgggg cggcagctcc |
| 3181 | ccagcaggac taccccgacc ctgggtcttg aggaagtgct gagcagcagg gactgtcacc |
| 3241 | ctgccctgcc gcttcctccc ggcttccatc cccacccggg gcccaattac ccatccttcc |
| 3301 | tgcccgatca gatgcagccg caagtcccgc cgctccatta ccaagagctc atgccacccg |
| 3361 | gttcctgcat gccagaggag cccaagccaa agaggggaag acgatcgtgg ccccggaaaa |
| 3421 | ggaccgccac ccacacttgt gattacgcgg gctgcggcaa aacctacaca aagagttccc |
| 3481 | atctcaaggc acacctgcga acccacacag gtgagaaacc ttaccactgt gactgggacg |
| 3541 | gctgtggatg gaaattcgcc cgctcagatg aactgaccag gcactaccgt aaacacacgg |
| 3601 | ggcaccgccc gttccagtgc caaaaatgcg accgagcatt ttccaggtcg gaccacctcg |
| 3661 | ccttacacat gaagaggcat ttttaaatcc cagacagtgg atatgaccca cactgccaga |
| 3721 | agagaattcc tgcaggcctc gagggccggc gcgccgcggc cgcgactcta gaatttcgac |
| 3781 | ctcgacatta attccggtta ttttccacca tattgccgtc ttttggcaat gtgagggccc |
| 3841 | ggaaacctgg ccctgtcttc ttgacgagca ttcctagggg tctttcccct ctcgccaaag |
| 3901 | gaatgcaagg tctgttgaat gtcgtgaagg aagcagttcc tctggaagct tcttgaagac |
| 3961 | aaacaacgtc tgtagcgacc ctttgcaggc agcggaaccc cccacctggc gacaggtgcc |
| 4021 | tctgcggcca aaagccacgt gtataagata cacctgcaaa ggcggcacaa ccccagtgcc |
| 4081 | acgttgtgag ttggatagtt gtggaaagag tcaaatggct ctcctcaagc gtattcaaca |
| 4141 | aggggctgaa ggatgcccag aaggtacccc attgtatggg atctgatctg gggcctcggt |
| 4201 | gcacatgctt tacatgtgtt tagtcgaggt taaaaaacgt ctaggccccc cgaaccacgg |
| 4261 | ggacgtggtt ttcctttgaa aaacacgatg ataataccat ggcttcgtac ccctgccatc |
| 4321 | aacacgcgtc tgcgttcgac caggctgcgc gttctcgcgg ccataacaac cgacgtacgg |
| 4381 | cgttgcgccc tcgccggcaa caaaaagcca cggaagtccg cctggagcag aaaatgccca |
| 4441 | cgctactgcg ggtttatata gacggtcccc acgggatggg gaaaaccacc accacgcaac |
| 4501 | tgctggtggc cctgggttcg cgcgacgata tcgtctacgt acccgagccg atgacttact |
| 4561 | ggcgggtgtt gggggcttcc gagacaatcg cgaacatcta caccacacaa caccgcctcg |
| 4621 | accagggtga gatatcggcc ggggacgcgg cggtggtaat gacaagcgcc cagataacaa |
| 4681 | tgggcatgcc ttatgccgtg accgacgccg ttctggctcc tcatatcggg ggggaggctg |
| 4741 | ggagctcaca tgccccgccc ccggccctca ccctcatctt cgaccgccat cccatcgccg |
| 4801 | ccctcctgtg ctacccggcc gcgcgatacc ttatgggcag catgaccccc caggccgtgc |
| 4861 | tggcgttcgt ggccctcatc ccgccgacct tgcccggcac aaacatcgtg ttgggggccc |
| 4921 | ttccggagga cagacacatc gaccgcctgg ccaaacgcca gcgccccggc gagcggcttg |
| 4981 | acctggctat gctggccgcg tcgccgcgtt tatgggctgc ttgccaatac ggtgcggtat |
| 5041 | ctgcagggcg gcgggtcgtg gcgggaggat tggggacagc tttcgggggc ggccgtgccg |
| 5101 | ccccagggtg ccgagcccca gagcaacgcg ggcccacgac cccatatcgg ggacacgtta |
| 5161 | tttaccctgt ttcgggcccc cgagttgctg gcccccaacg gcgacctgta taacgtgttt |
| 5221 | gcctgggctt tggctcgacg gtacctttaa gaccaatgac ttacaaggca gctgtagatc |
| 5281 | aattcgatat caagcttatc gataatcaac ctctggatta caaaatttgt gaaagattga |
| 5341 | ctggtattct taactatgtt gctcctttta cgctatgtgg atacgctgct ttaatgcctt |
| 5401 | tgtatcatgc tattgcttcc cgtatggctt tcattttctc ctccttgtat aaatcctggt |
| 5461 | tgctgtctct ttatgaggag ttgtggcccg ttgtcaggca acgtggcgtg gtgtgcactg |
| 5521 | tgtttgctga cgcaaccccc actggttggg gcattgccac cacctgtcag ctcctttccg |
| 5581 | ggactttcgc tttccccctc cctattgcca cggcggaact catcgccgcc tgccttgccc |
| 5641 | gctgctggac aggggctcgg ctgttgggca ctgacaattc cgtggtgttg tcggggaaat |
| 5701 | catcgtcctt tccttggctg ctcgcctgtg ttgccacctg gattctgcgc gggacgtcct |
| 5761 | tctgctacgt cccttcggcc ctcaatccag cggaccttcc ttcccgcggc ctgctgccgg |
| 5821 | ctctgcggcc tcttccgcgt cttcgccttc gccctcagac gagtcggatc tccctttggg |
| 5881 | ccgcctcccc gcatcgatac cgtcgacgat aaaataaaag attttattta gtctccagaa |
| 5941 | aaagggggga atgaaagacc ccacctgtag gtttggcaag ctagcttaag taacgccatt |
| 6001 | ttgcaaggca tggaaaaata cataactgag aatagagaag ttcagatcaa ggtcaggaac |
| 6061 | agatggaaca gctgaatatg ggccaaacag gatatctgtg gtaagcagtt cctgccccgg |
| 6121 | ctcagggcca agaacagatg gaacagctga atatgggcca aacaggatat ctgtggtaag |
| 6181 | cagttcctgc cccggctcag ggccaagaac agatggtccc cagatgcggt ccagccctca |
| 6241 | gcagtttcta gagaaccatc agatgtttcc agggtgcccc aaggacctga aatgaccctg |
| 6301 | tgccttattt gaactaacca atcagttcgc ttctcgcttc tgttcgcgcg cttctgctcc |
| 6361 | ccgagctcaa taaaagagcc cacaacccct cactcggggc gccagtcctc cgattgactg |
| 6421 | agtcgcccgg gtacccgtgt atccaataaa ccctcttgca gttgcatccg acttgtggtc |
| 6481 | tcgctgttcc ttgggagggt ctcctctgag tgattgacta cccgtcagcg ggggtctttc |
| 6541 | acatgcagca tgtatcaaaa ttaatttggt tttttttctt aagtatttac attaaatggc |
| 6601 | catagttgca ttaatgaatc ggccaacgcg cggggagagg cggtttgcgt attgggcgct |
| 6661 | cttccgcttc ctcgctcact gactcgctgc gctcggtcgt tcggctgcgg cgagcggtat |
| 6721 | cagctcactc aaaggcggta atacggttat ccacagaatc aggggataac gcaggaaaga |
| 6781 | acatgtgagc aaaaggccag caaaaggcca ggaaccgtaa aaaggccgcg ttgctggcgt |
| 6841 | ttttccatag gctccgcccc cctgacgagc atcacaaaaa tcgacgctca agtcagaggt |
| 6901 | ggcgaaaccc gacaggacta taaagatacc aggcgtttcc ccctggaagc tccctcgtgc |
| 6961 | gctctcctgt tccgaccctg ccgcttaccg gatacctgtc cgcctttctc ccttcgggaa |
| 7021 | gcgtggcgct ttctcatagc tcacgctgta ggtatctcag ttcggtgtag gtcgttcgct |
| 7081 | ccaagctggg ctgtgtgcac gaaccccccg ttcagcccga ccgctgcgcc ttatccggta |
| 7141 | actatcgtct tgagtccaac ccggtaagac acgacttatc gccactggca gcagccactg |
| 7201 | gtaacaggat tagcagagcg aggtatgtag gcggtgctac agagttcttg aagtggtggc |
| 7261 | ctaactacgg ctacactaga agaacagtat ttggtatctg cgctctgctg aagccagtta |
| 7321 | ccttcggaaa aagagttggt agctcttgat ccggcaaaca aaccaccgct ggtagcggtg |
| 7381 | gtttttttgt ttgcaagcag cagattacgc gcagaaaaaa aggatctcaa gaagatcctt |
| 7441 | tgatcttttc tacggggtct gacgctcagt ggaacgaaaa ctcacgttaa gggattttgg |
| 7501 | tcatgagatt atcaaaaagg atcttcacct agatcctttt gcggccggcc gcaaatcaat |
| 7561 | ctaaagtata tatgagtaaa cttggtctga cagttaccaa tgcttaatca gtgaggcacc |
| 7621 | tatctcagcg atctgtctat ttcgttcatc catagttgcc tgactccccg tcgtgtagat |
| 7681 | aactacgata cgggagggct taccatctgg ccccagtgct gcaatgatac cgcgagaccc |
| 7741 | acgctcaccg gctccagatt tatcagcaat aaaccagcca gccggaaggg ccgagcgcag |
| 7801 | aagtggtcct gcaactttat ccgcctccat ccagtctatt aattgttgcc gggaagctag |
| 7861 | agtaagtagt tcgccagtta atagtttgcg caacgttgtt gccattgcta caggcatcgt |
| 7921 | ggtgtcacgc tcgtcgtttg gtatggcttc attcagctcc ggttcccaac gatcaaggcg |
| 7981 | agttacatga tcccccatgt tgtgcaaaaa agcggttagc tccttcggtc ctccgatcgt |
| 8041 | tgtcagaagt aagttggccg cagtgttatc actcatggtt atggcagcac tgcataattc |
| 8101 | tcttactgtc atgccatccg taagatgctt ttctgtgact ggtgagtact caaccaagtc |
| 8161 | attctgagaa tagtgtatgc ggcgaccgag ttgctcttgc ccggcgtcaa tacgggataa |
| 8221 | taccgcgcca catagcagaa ctttaaaagt gctcatcatt ggaaaacgtt cttcggggcg |
| 8281 | aaaactctca aggatcttac cgctgttgag atccagttcg atgtaaccca ctcgtgcacc |
| 8341 | caactgatct tcagcatctt ttactttcac cagcgtttct gggtgagcaa aaacaggaag |
| 8401 | gcaaaatgcc gcaaaaaagg gaataagggc gacacggaaa tgttgaatac tcatactctt |
| 8461 | cctttttcaa tattattgaa gcatttatca gggttattgt ctcatgagcg gatacatatt |
| 8521 | tgaatgtatt tagaaaaata aacaaatagg ggttccgcgc acatttc |

pMXs-c-Myc-IRES-TK

| (SEQ ID NO: 36) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aatcgacggt |
| 1921 | atcgataagc ttgatatctg cggcctagct agccgcgacg atgcccctca acgttagctt |
| 1981 | caccaacagg aactatgacc tcgactacga ctcggtgcag ccgtatttct actgcgacga |
| 2041 | ggaggagaac ttctaccagc agcagcagca gagcgagctg cagcccccgg cgcccagcga |
| 2101 | ggatatctgg aagaaattcg agctgctgcc caccccgccc ctgtccccta gccgccgctc |
| 2161 | cgggctctgc tcgccctcct acgttgcggt cacacccttc tcccttcggg gagacaacga |
| 2221 | cggcggtggc gggagcttct ccacggccga ccagctggag atggtgaccg agctgctggg |
| 2281 | aggagacatg gtgaaccaga gtttcatctg cgacccggac gacgagacct tcatcaaaaa |
| 2341 | catcatcatc caggactgta tgtggagcgg cttctcggcc gccgccaagc tcgtctcaga |
| 2401 | gaagctggcc tcctaccagg ctgcgcgcaa agacagcggc agcccgaacc ccgcccgcgg |
| 2461 | ccacagcgtc tgctccacct ccagcttgta cctgcaggat ctgagcgccg ccgcctcaga |
| 2521 | gtgcatcgac ccctcggtgg tcttccccta ccctctcaac gacagcagct cgcccaagtc |
| 2581 | ctgcgcctcg caagactcca gcgccttctc tccgtcctcg gattctctgc tctcctcgac |
| 2641 | ggagtcctcc ccgcagggca gccccgagcc cctggtgctc catgaggaga caccgcccac |
| 2701 | caccagcagc gactctgagg aggaacaaga agatgaggaa gaaatcgatg ttgtttctgt |
| 2761 | ggaaaagagg caggctcctg gcaaaaggtc agagtctgga tcaccttctg ctggaggcca |
| 2821 | cagcaaacct cctcacagcc cactggtcct caagaggtgc cacgtctcca cacatcagca |
| 2881 | caactacgca gcgcctccct ccactcggaa ggactatcct gctgccaaga gggtcaagtt |
| 2941 | ggacagtgtc agagtcctga gacagatcag caacaaccga aaatgcacca gccccaggtc |
| 3001 | ctcggacacc gaggagaatg tcaagaggcg aacacacaac gtcttggagc gccagaggag |
| 3061 | gaacgagcta aaacggagct tttttgccct gcgtgaccag atcccggagt tggaaaacaa |
| 3121 | tgaaaaggcc cccaaggtag ttatccttaa aaaagccaca gcatacatcc tgtccgtcca |
| 3181 | agcagaggag caaaagctca tttctgaaga ggacttgttg cggaaacgac gagaacagtt |
| 3241 | gaaacacaaa cttgaacagc tacggaactc ttgtgcgtaa ggaaaagtaa ggaaaacgat |
| 3301 | tccttctaac agaaatgtcc tgagcggccg cgactctaga atttcgacct cgacattaat |
| 3361 | tccggttatt ttccaccata ttgccgtctt ttggcaatgt gagggcccgg aaacctggcc |
| 3421 | ctgtcttctt gacgagcatt cctaggggtc tttcccctct cgccaaagga atgcaaggtc |
| 3481 | tgttgaatgt cgtgaaggaa gcagttcctc tggaagcttc ttgaagacaa acaacgtctg |
| 3541 | tagcgaccct ttgcaggcag cggaaccccc cacctggcga caggtgcctc tgcggccaaa |
| 3601 | agccacgtgt ataagataca cctgcaaagg cggcacaacc ccagtgccac gttgtgagtt |
| 3661 | ggatagttgt ggaaagagtc aaatggctct cctcaagcgt attcaacaag gggctgaagg |
| 3721 | atgcccagaa ggtaccccat tgtatgggat ctgatctggg gcctcggtgc acatgcttta |
| 3781 | catgtgttta gtcgaggtta aaaaacgtct aggccccccg aaccacgggg acgtggtttt |
| 3841 | cctttgaaaa acacgatgat aataccatgg cttcgtaccc ctgccatcaa cacgcgtctg |
| 3901 | cgttcgacca ggctgcgcgt tctcgcggcc ataacaaccg acgtacggcg ttgcgccctc |
| 3961 | gccggcaaca aaaagccacg gaagtccgcc tggagcagaa aatgcccacg ctactgcggg |
| 4021 | tttatataga cggtccccac gggatgggga aaaccaccac cacgcaactg ctggtggccc |
| 4081 | tgggttcgcg cgacgatatc gtctacgtac ccgagccgat gacttactgg cgggtgttgg |
| 4141 | gggcttccga gacaatcgcg aacatctaca ccacacaaca ccgcctcgac cagggtgaga |
| 4201 | tatcggccgg ggacgcggcg gtggtaatga caagcgccca gataacaatg ggcatgcctt |
| 4261 | atgccgtgac cgacgccgtt ctggctcctc atatcggggg ggaggctggg agctcacatg |
| 4321 | ccccgccccc ggccctcacc ctcatcttcg accgccatcc catcgccgcc ctcctgtgct |
| 4381 | acccggccgc gcgatacctt atgggcagca tgacccccca ggccgtgctg gcgttcgtgg |
| 4441 | ccctcatccc gccgaccttg cccggcacaa acatcgtgtt gggggccctt ccggaggaca |
| 4501 | gacacatcga ccgcctggcc aaacgccagc gccccggcga gcggcttgac ctggctatgc |
| 4561 | tggccgcgtc gccgcgttta tgggctgctt gccaatacgg tgcggtatct gcagggcggc |
| 4621 | gggtcgtggc gggaggattg gggacagctt tcgggggcgg ccgtgccgcc ccagggtgcc |
| 4681 | gagccccaga gcaacgcggg cccacgaccc catatcgggg acacgttatt taccctgttt |
| 4741 | cgggcccccg agttgctggc ccccaacggc gacctgtata acgtgtttgc ctgggctttg |
| 4801 | gctcgacggt acctttaaga ccaatgactt acaaggcagc tgtagatcaa ttcgatatca |
| 4861 | agcttatcga taatcaacct ctggattaca aaatttgtga aagattgact ggtattctta |
| 4921 | actatgttgc tccttttacg ctatgtggat acgctgcttt aatgcctttg tatcatgcta |
| 4981 | ttgcttcccg tatggctttc attttctcct ccttgtataa atcctggttg ctgtctcttt |
| 5041 | atgaggagtt gtggcccgtt gtcaggcaac gtggcgtggt gtgcactgtg tttgctgacg |
| 5101 | caacccccac tggttggggc attgccacca cctgtcagct cctttccggg actttcgctt |
| 5161 | tccccctccc tattgccacg gcggaactca tcgccgcctg ccttgcccgc tgctggacag |
| 5221 | gggctcggct gttgggcact gacaattccg tggtgttgtc ggggaaatca tcgtcctttc |
| 5281 | cttggctgct cgcctgtgtt gccacctgga ttctgcgcgg gacgtccttc tgctacgtcc |
| 5341 | cttcggccct caatccagcg gaccttcctt cccgcggcct gctgccggct ctgcggcctc |
| 5401 | ttccgcgtct tcgccttcgc cctcagacga gtcggatctc cctttgggcc gcctccccgc |
| 5461 | atcgataccg tcgacgataa aataaaagat tttatttagt ctccagaaaa aggggggaat |
| 5521 | gaaagacccc acctgtaggt ttggcaagct agcttaagta acgccatttt gcaaggcatg |
| 5581 | gaaaaataca taactgagaa tagagaagtt cagatcaagg tcaggaacag atggaacagc |
| 5641 | tgaatatggg ccaaacagga tatctgtggt aagcagttcc tgccccggct cagggccaag |
| 5701 | aacagatgga acagctgaat atgggccaaa caggatatct gtggtaagca gttcctgccc |
| 5761 | cggctcaggg ccaagaacag atggtcccca gatgcggtcc agccctcagc agtttctaga |
| 5821 | gaaccatcag atgtttccag ggtgccccaa ggacctgaaa tgaccctgtg ccttatttga |
| 5881 | actaaccaat cagttcgctt ctcgcttctg ttcgcgcgct tctgctcccc gagctcaata |
| 5941 | aaagagccca caacccctca ctcggggcgc cagtcctccg attgactgag tcgcccgggt |
| 6001 | acccgtgtat ccaataaacc ctcttgcagt tgcatccgac ttgtggtctc gctgttcctt |
| 6061 | gggagggtct cctctgagtg attgactacc cgtcagcggg ggtctttcac atgcagcatg |
| 6121 | tatcaaaatt aatttggttt tttttcttaa gtatttacat taaatggcca tagttgcatt |
| 6181 | aatgaatcgg ccaacgcgcg gggagaggcg gtttgcgtat tgggcgctct tccgcttcct |
| 6241 | cgctcactga ctcgctgcgc tcggtcgttc ggctgcggcg agcggtatca gctcactcaa |
| 6301 | aggcggtaat acggttatcc acagaatcag gggataacgc aggaaagaac atgtgagcaa |
| 6361 | aaggccagca aaaggccagg aaccgtaaaa aggccgcgtt gctggcgttt ttccataggc |
| 6421 | tccgcccccc tgacgagcat cacaaaaatc gacgctcaag tcagaggtgg cgaaacccga |
| 6481 | caggactata aagataccag gcgtttcccc ctggaagctc cctcgtgcgc tctcctgttc |
| 6541 | cgaccctgcc gcttaccgga tacctgtccg cctttctccc ttcgggaagc gtggcgcttt |
| 6601 | ctcatagctc acgctgtagg tatctcagtt cggtgtaggt cgttcgctcc aagctgggct |
| 6661 | gtgtgcacga accccccgtt cagcccgacc gctgcgcctt atccggtaac tatcgtcttg |
| 6721 | agtccaaccc ggtaagacac gacttatcgc cactggcagc agccactggt aacaggatta |
| 6781 | gcagagcgag gtatgtaggc ggtgctacag agttcttgaa gtggtggcct aactacggct |
| 6841 | acactagaag aacagtattt ggtatctgcg ctctgctgaa gccagttacc ttcggaaaaa |
| 6901 | gagttggtag ctcttgatcc ggcaaacaaa ccaccgctgg tagcggtggt ttttttgttt |
| 6961 | gcaagcagca gattacgcgc agaaaaaaag gatctcaaga agatcctttg atcttttcta |
| 7021 | cggggtctga cgctcagtgg aacgaaaact cacgttaagg gattttggtc atgagattat |
| 7081 | caaaaaggat cttcacctag atccttttgc ggccggccgc aaatcaatct aaagtatata |
| 7141 | tgagtaaact tggtctgaca gttaccaatg cttaatcagt gaggcaccta tctcagcgat |
| 7201 | ctgtctattt cgttcatcca tagttgcctg actccccgtc gtgtagataa ctacgatacg |
| 7261 | ggagggctta ccatctggcc ccagtgctgc aatgataccg cgagacccac gctcaccggc |
| 7321 | tccagattta tcagcaataa accagccagc cggaagggcc gagcgcagaa gtggtcctgc |
| 7381 | aactttatcc gcctccatcc agtctattaa ttgttgccgg gaagctagag taagtagttc |
| 7441 | gccagttaat agtttgcgca acgttgttgc cattgctaca ggcatcgtgg tgtcacgctc |
| 7501 | gtcgtttggt atggcttcat tcagctccgg ttcccaacga tcaaggcgag ttacatgatc |
| 7561 | ccccatgttg tgcaaaaaag cggttagctc cttcggtcct ccgatcgttg tcagaagtaa |
| 7621 | gttggccgca gtgttatcac tcatggttat ggcagcactg cataattctc ttactgtcat |
| 7681 | gccatccgta agatgctttt ctgtgactgg tgagtactca accaagtcat tctgagaata |
| 7741 | gtgtatgcgg cgaccgagtt gctcttgccc ggcgtcaata cgggataata ccgcgccaca |
| 7801 | tagcagaact ttaaaagtgc tcatcattgg aaaacgttct tcggggcgaa aactctcaag |
| 7861 | gatcttaccg ctgttgagat ccagttcgat gtaacccact cgtgcaccca actgatcttc |
| 7921 | agcatctttt actttcacca gcgtttctgg gtgagcaaaa acaggaaggc aaaatgccgc |
| 7981 | aaaaaaggga ataagggcga cacggaaatg ttgaatactc atactcttcc tttttcaata |
| 8041 | ttattgaagc atttatcagg gttattgtct catgagcgga tacatatttg aatgtattta |
| 8101 | gaaaaataaa caaatagggg ttccgcgcac atttc |

pMXs-IRES-EGFP

| (SEQ ID NO: 37) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | cccagtgtgg tggtacggga attcctgcag gcctcgaggg ccggcgcgcc gcggccgcta |
| 1981 | cgtaaattcc gcccctctcc ctcccccccc cctaacgtta ctggccgaag ccgcttggaa |
| 2041 | taaggccggt gtgcgtttgt ctatatgtta ttttccacca tattgccgtc ttttggcaat |
| 2101 | gtgagggccc ggaaacctgg ccctgtcttc ttgacgagca ttcctagggg tctttcccct |
| 2161 | ctcgccaaag gaatgcaagg tctgttgaat gtcgtgaagg aagcagttcc tctggaagct |
| 2221 | tcttgaagac aaacaacgtc tgtagcgacc ctttgcaggc agcggaaccc cccacctggc |
| 2281 | gacaggtgcc tctgcggcca aaagccacgt gtataagata cacctgcaaa ggcggcacaa |
| 2341 | ccccagtgcc acgttgtgag ttggatagtt gtggaaagag tcaaatggct ctcctcaagc |
| 2401 | gtattcaaca aggggctgaa ggatgcccag aaggtacccc attgtatggg atctgatctg |
| 2461 | gggcctcggt gcacatgctt tacatgtgtt tagtcgaggt taaaaaaacg tctaggcccc |
| 2521 | ccgaaccacg gggacgtggt tttcctttga aaaacacgat gataatatgg ccacaaccat |
| 2581 | ggtgagcaag ggcgaggagc tgttcaccgg ggtggtgccc atcctggtcg agctggacgg |
| 2641 | cgacgtaaac ggccacaagt tcagcgtgtc cggcgagggc gagggcgatg ccacctacgg |
| 2701 | caagctgacc ctgaagttca tctgcaccac cggcaagctg cccgtgccct ggcccaccct |
| 2761 | cgtgaccacc ctgacctacg gcgtgcagtg cttcagccgc taccccgacc acatgaagca |
| 2821 | gcacgacttc ttcaagtccg ccatgcccga aggctacgtc caggagcgca ccatcttctt |
| 2881 | caaggacgac ggcaactaca agacccgcgc cgaggtgaag ttcgagggcg acaccctggt |
| 2941 | gaaccgcatc gagctgaagg gcatcgactt caaggaggac ggcaacatcc tggggcacaa |
| 3001 | gctggagtac aactacaaca gccacaacgt ctatatcatg gccgacaagc agaagaacgg |
| 3061 | catcaaggtg aacttcaaga tccgccacaa catcgaggac ggcagcgtgc agctcgccga |
| 3121 | ccactaccag cagaacaccc ccatcggcga cggccccgtg ctgctgcccg acaaccacta |
| 3181 | cctgagcacc cagtccgccc tgagcaaaga ccccaacgag aagcgcgatc acatggtcct |
| 3241 | gctggagttc gtgaccgccg ccgggatcac tctcggcatg gacgagctgt acaagtaagt |
| 3301 | cgacgataaa ataaaagatt ttatttagtc tccagaaaaa ggggggaatg aaagacccca |
| 3361 | cctgtaggtt tggcaagcta gcttaagtaa cgccattttg caaggcatgg aaaaatacat |
| 3421 | aactgagaat agagaagttc agatcaaggt caggaacaga tggaacagct gaatatgggc |
| 3481 | caaacaggat atctgtggta agcagttcct gccccggctc agggccaaga acagatggaa |
| 3541 | cagctgaata tgggccaaac aggatatctg tggtaagcag ttcctgcccc ggctcagggc |
| 3601 | caagaacaga tggtccccag atgcggtcca gccctcagca gtttctagag aaccatcaga |
| 3661 | tgtttccagg gtgccccaag gacctgaaat gaccctgtgc cttatttgaa ctaaccaatc |
| 3721 | agttcgcttc tcgcttctgt tcgcgcgctt ctgctccccg agctcaataa aagagcccac |
| 3781 | aacccctcac tcggggcgcc agtcctccga ttgactgagt cgcccgggta cccgtgtatc |
| 3841 | caataaaccc tcttgcagtt gcatccgact tgtggtctcg ctgttccttg ggagggtctc |
| 3901 | ctctgagtga ttgactaccc gtcagcgggg gtctttcaca tgcagcatgt atcaaaatta |
| 3961 | atttggtttt ttttcttaag tatttacatt aaatggccat agttgcatta atgaatcggc |
| 4021 | caacgcgcgg ggagaggcgg tttgcgtatt gggcgctctt ccgcttcctc gctcactgac |
| 4081 | tcgctgcgct cggtcgttcg gctgcggcga gcggtatcag ctcactcaaa ggcggtaata |
| 4141 | cggttatcca cagaatcagg ggataacgca ggaaagaaca tgtgagcaaa aggccagcaa |
| 4201 | aaggccagga accgtaaaaa ggccgcgttg ctggcgtttt tccataggct ccgcccccct |
| 4261 | gacgagcatc acaaaaatcg acgctcaagt cagaggtggc gaaacccgac aggactataa |
| 4321 | agataccagg cgtttccccc tggaagctcc ctcgtgcgct ctcctgttcc gaccctgccg |
| 4381 | cttaccggat acctgtccgc ctttctccct tcgggaagcg tggcgctttc tcatagctca |
| 4441 | cgctgtaggt atctcagttc ggtgtaggtc gttcgctcca agctgggctg tgtgcacgaa |
| 4501 | ccccccgttc agcccgaccg ctgcgcctta tccggtaact atcgtcttga gtccaacccg |
| 4561 | gtaagacacg acttatcgcc actggcagca gccactggta acaggattag cagagcgagg |
| 4621 | tatgtaggcg gtgctacaga gttcttgaag tggtggccta actacggcta cactagaaga |
| 4681 | acagtatttg gtatctgcgc tctgctgaag ccagttacct tcggaaaaag agttggtagc |
| 4741 | tcttgatccg gcaaacaaac caccgctggt agcggtggtt tttttgtttg caagcagcag |
| 4801 | attacgcgca gaaaaaaagg atctcaagaa gatcctttga tcttttctac ggggtctgac |
| 4861 | gctcagtgga acgaaaactc acgttaaggg attttggtca tgagattatc aaaaaggatc |
| 4921 | ttcacctaga tccttttgcg gccggccgca aatcaatcta aagtatatat gagtaaactt |
| 4981 | ggtctgacag ttaccaatgc ttaatcagtg aggcacctat ctcagcgatc tgtctatttc |
| 5041 | gttcatccat agttgcctga ctccccgtcg tgtagataac tacgatacgg gagggcttac |
| 5101 | catctggccc cagtgctgca atgataccgc gagacccacg ctcaccggct ccagatttat |
| 5161 | cagcaataaa ccagccagcc ggaagggccg agcgcagaag tggtcctgca actttatccg |
| 5221 | cctccatcca gtctattaat tgttgccggg aagctagagt aagtagttcg ccagttaata |
| 5281 | gtttgcgcaa cgttgttgcc attgctacag gcatcgtggt gtcacgctcg tcgtttggta |
| 5341 | tggcttcatt cagctccggt tcccaacgat caaggcgagt tacatgatcc cccatgttgt |
| 5401 | gcaaaaaagc ggttagctcc ttcggtcctc cgatcgttgt cagaagtaag ttggccgcag |
| 5461 | tgttatcact catggttatg gcagcactgc ataattctct tactgtcatg ccatccgtaa |
| 5521 | gatgcttttc tgtgactggt gagtactcaa ccaagtcatt ctgagaatag tgtatgcggc |
| 5581 | gaccgagttg ctcttgcccg gcgtcaatac gggataatac cgcgccacat agcagaactt |
| 5641 | taaaagtgct catcattgga aaacgttctt cggggcgaaa actctcaagg atcttaccgc |
| 5701 | tgttgagatc cagttcgatg taacccactc gtgcacccaa ctgatcttca gcatctttta |
| 5761 | ctttcaccag cgtttctggg tgagcaaaaa caggaaggca aaatgccgca aaaaagggaa |
| 5821 | taagggcgac acggaaatgt tgaatactca tactcttcct ttttcaatat tattgaagca |
| 5881 | tttatcaggg ttattgtctc atgagcggat acatatttga atgtatttag aaaaataaac |
| 5941 | aaataggggt tccgcgcaca tttc |

pMXs-Oct4-IRES-Hygro

| (SEQ ID NO: 38) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | ccgaattccc ttcgcaagcc ctcatttcac caggcccccg gcttggggcg ccttccttcc |
| 1981 | ccatggcggg acacctggct tcggatttcg ccttctcgcc ccctccaggt ggtggaggtg |
| 2041 | atgggccagg ggggccggag ccgggctggg ttgatcctcg gacctggcta agcttccaag |
| 2101 | gccctcctgg agggccagga atcgggccgg gggttgggcc aggctctgag gtgtggggga |
| 2161 | ttcccccatg ccccccgccg tatgagttct gtggggggat ggcgtactgt gggccccagg |
| 2221 | ttggagtggg gctagtgccc caaggcggct tggagacctc tcagcctgag ggcgaagcag |
| 2281 | gagtcggggt ggagagcaac tccgatgggg cctccccgga gccctgcacc gtcacccctg |
| 2341 | gtgccgtgaa gctggagaag gagaagctgg agcaaaaccc ggaggagtcc caggacatca |
| 2401 | aagctctgca gaaagaactc gagcaatttg ccaagctcct gaagcagaag aggatcaccc |
| 2461 | tgggatatac acaggccgat gtggggctca ccctgggggt tctatttggg aaggtattca |
| 2521 | gccaaacgac catctgccgc tttgaggctc tgcagcttag cttcaagaac atgtgtaagc |
| 2581 | tgcggccctt gctgcagaag tgggtggagg aagctgacaa caatgaaaat cttcaggaga |
| 2641 | tatgcaaagc agaaaccctc gtgcaggccc gaaagagaaa gcgaaccagt atcgagaacc |
| 2701 | gagtgagagg caacctggag aatttgttcc tgcagtgccc gaaacccaca ctgcagcaga |
| 2761 | tcagccacat cgcccagcag cttgggctcg agaaggatgt ggtccgagtg tggttctgta |
| 2821 | accggcgcca gaagggcaag cgatcaagca gcgactatgc acaacgagag gattttgagg |
| 2881 | ctgctgggtc tcctttctca gggggaccag tgtcctttcc tctggcccca gggccccatt |
| 2941 | ttggtacccc aggctatggg agccctcact tcactgcact gtactcctcg gtccctttcc |
| 3001 | ctgaggggga agcctttccc cctgtctccg tcaccactct gggctctccc atgcattcaa |
| 3061 | actgaggtgc ctgcccttct aggaatgggg gacaggggga ggggaggagc tagggaagaa |
| 3121 | ttcgcggcgg ccgctacgta aattccgccc ctctccctcc ccccccccta acgttactgg |
| 3181 | ccgaagccgc ttggaataag gccggtgtgc gtttgtctat atgttatttt ccaccatatt |
| 3241 | gccgtctttt ggcaatgtga gggcccggaa acctggccct gtcttcttga cgagcattcc |
| 3301 | taggggtctt tcccctctcg ccaaaggaat gcaaggtctg ttgaatgtcg tgaaggaagc |
| 3361 | agttcctctg gaagcttctt gaagacaaac aacgtctgta gcgacccttt gcaggcagcg |
| 3421 | gaacccccca cctggcgaca ggtgcctctg cggccaaaag ccacgtgtat aagatacacc |
| 3481 | tgcaaaggcg gcacaacccc agtgccacgt tgtgagttgg atagttgtgg aaagagtcaa |
| 3541 | atggctctcc tcaagcgtat tcaacaaggg gctgaaggat gcccagaagg taccccattg |
| 3601 | tatgggatct gatctggggc ctcggtgcac atgctttaca tgtgtttagt cgaggttaaa |
| 3661 | aaaacgtcta ggccccccga accacgggga cgtggttttc ctttgaaaaa cacgatgata |
| 3721 | atatggccac aaccatgtat gaaaaagcct gaactcaccg cgacgtctgt cgagaagttt |
| 3781 | ctgatcgaaa agttcgacag cgtctccgac ctgatgcagc tctcggaggg cgaagaatct |
| 3841 | cgtgctttca gcttcgatgt aggagggcgt ggatatgtcc tgcgggtaaa tagctgcgcc |
| 3901 | gatggtttct acaaagatcg ttatgtttat cggcactttg catcggccgc gctcccgatt |
| 3961 | ccggaagtgc ttgacattgg ggaattcagc gagagcctga cctattgcat ctcccgccgt |
| 4021 | gcacagggtg tcacgttgca agacctgcct gaaaccgaac tgcccgctgt tctgcaaccc |
| 4081 | gtcgcggagc tcatggatgc gatcgctgcg gccgatctta gccagacgag cgggttcggc |
| 4141 | ccattcggac cgcaaggaat cggtcaatac actacatggc gtgatttcat atgcgcgatt |
| 4201 | gctgatcccc atgtgtatca ctggcaaact gtgatggacg acaccgtcag tgcgtccgtc |
| 4261 | gcgcaggctc tcgatgagct gatgctttgg gccgaggact gccccgaagt ccggcacctc |
| 4321 | gtgcacgcgg atttcggctc caacaatgtc ctgacggaca atggccgcat aacagcggtc |
| 4381 | attgactgga gcgaggcgat gttcggggat tcccaatacg aggtcgccaa catcttcttc |
| 4441 | tggaggccgt ggttggcttg tatggagcag cagacgcgct acttcgagcg gaggcatccg |
| 4501 | gagcttgcag gatcgccgcg gctccgggcg tatatgctcc gcattggtct tgaccaactc |
| 4561 | tatcagagct tggttgacgg caatttcgat gatgcagctt gggcgcaggg tcgatgcgac |
| 4621 | gcaatcgtcc gatccggagc cgggactgtc gggcgtacac aaatcgcccg cagaagcgcg |
| 4681 | gccgtctgga ccgatggctg tgtagaagta ctcgccgata gtggaaaccg acgccccagc |
| 4741 | actcgtccga gggcaaagga atgagtcgag aattcggtcg acgataaaat aaaagatttt |
| 4801 | atttagtctc cagaaaaagg ggggaatgaa agaccccacc tgtaggtttg gcaagctagc |
| 4861 | ttaagtaacg ccattttgca aggcatggaa aaatacataa ctgagaatag agaagttcag |
| 4921 | atcaaggtca ggaacagatg gaacagctga atatgggcca aacaggatat ctgtggtaag |
| 4981 | cagttcctgc cccggctcag ggccaagaac agatggaaca gctgaatatg ggccaaacag |
| 5041 | gatatctgtg gtaagcagtt cctgccccgg ctcagggcca agaacagatg gtccccagat |
| 5101 | gcggtccagc cctcagcagt ttctagagaa ccatcagatg tttccagggt gccccaagga |
| 5161 | cctgaaatga ccctgtgcct tatttgaact aaccaatcag ttcgcttctc gcttctgttc |
| 5221 | gcgcgcttct gctccccgag ctcaataaaa gagcccacaa cccctcactc ggggcgccag |
| 5281 | tcctccgatt gactgagtcg cccgggtacc cgtgtatcca ataaaccctc ttgcagttgc |
| 5341 | atccgacttg tggtctcgct gttccttggg agggtctcct ctgagtgatt gactacccgt |
| 5401 | cagcgggggt ctttcacatg cagcatgtat caaaattaat ttggtttttt ttcttaagta |
| 5461 | tttacattaa atggccatag ttgcattaat gaatcggcca acgcgcgggg agaggcggtt |
| 5521 | tgcgtattgg gcgctcttcc gcttcctcgc tcactgactc gctgcgctcg gtcgttcggc |
| 5581 | tgcggcgagc ggtatcagct cactcaaagg cggtaatacg gttatccaca gaatcagggg |
| 5641 | ataacgcagg aaagaacatg tgagcaaaag gccagcaaaa ggccaggaac cgtaaaaagg |
| 5701 | ccgcgttgct ggcgtttttc cataggctcc gcccccctga cgagcatcac aaaaatcgac |
| 5761 | gctcaagtca gaggtggcga aacccgacag gactataaag ataccaggcg tttccccctg |
| 5821 | gaagctccct cgtgcgctct cctgttccga ccctgccgct taccggatac ctgtccgcct |
| 5881 | ttctcccttc gggaagcgtg gcgctttctc atagctcacg ctgtaggtat ctcagttcgg |
| 5941 | tgtaggtcgt tcgctccaag ctgggctgtg tgcacgaacc ccccgttcag cccgaccgct |
| 6001 | gcgccttatc cggtaactat cgtcttgagt ccaacccggt aagacacgac ttatcgccac |
| 6061 | tggcagcagc cactggtaac aggattagca gagcgaggta tgtaggcggt gctacagagt |
| 6121 | tcttgaagtg gtggcctaac tacggctaca ctagaagaac agtatttggt atctgcgctc |
| 6181 | tgctgaagcc agttaccttc ggaaaaagag ttggtagctc ttgatccggc aaacaaacca |
| 6241 | ccgctggtag cggtggtttt tttgtttgca agcagcagat tacgcgcaga aaaaaaggat |
| 6301 | ctcaagaaga tcctttgatc ttttctacgg ggtctgacgc tcagtggaac gaaaactcac |
| 6361 | gttaagggat tttggtcatg agattatcaa aaaggatctt cacctagatc cttttgcggc |
| 6421 | cggccgcaaa tcaatctaaa gtatatatga gtaaacttgg tctgacagtt accaatgctt |
| 6481 | aatcagtgag gcacctatct cagcgatctg tctatttcgt tcatccatag ttgcctgact |
| 6541 | ccccgtcgtg tagataacta cgatacggga gggcttacca tctggcccca gtgctgcaat |
| 6601 | gataccgcga gacccacgct caccggctcc agatttatca gcaataaacc agccagccgg |
| 6661 | aagggccgag cgcagaagtg gtcctgcaac tttatccgcc tccatccagt ctattaattg |
| 6721 | ttgccgggaa gctagagtaa gtagttcgcc agttaatagt ttgcgcaacg ttgttgccat |
| 6781 | tgctacaggc atcgtggtgt cacgctcgtc gtttggtatg gcttcattca gctccggttc |
| 6841 | ccaacgatca aggcgagtta catgatcccc catgttgtgc aaaaaagcgg ttagctcctt |
| 6901 | cggtcctccg atcgttgtca gaagtaagtt ggccgcagtg ttatcactca tggttatggc |
| 6961 | agcactgcat aattctctta ctgtcatgcc atccgtaaga tgcttttctg tgactggtga |
| 7021 | gtactcaacc aagtcattct gagaatagtg tatgcggcga ccgagttgct cttgcccggc |
| 7081 | gtcaatacgg gataataccg cgccacatag cagaacttta aaagtgctca tcattggaaa |
| 7141 | acgttcttcg gggcgaaaac tctcaaggat cttaccgctg ttgagatcca gttcgatgta |
| 7201 | acccactcgt gcacccaact gatcttcagc atcttttact ttcaccagcg tttctgggtg |
| 7261 | agcaaaaaca ggaaggcaaa atgccgcaaa aaagggaata agggcgacac ggaaatgttg |
| 7321 | aatactcata ctcttccttt ttcaatatta ttgaagcatt tatcagggtt attgtctcat |
| 7381 | gagcggatac atatttgaat gtatttagaa aaataaacaa ataggggttc cgcgcacatt |
| 7441 | tc |

pMXs-Sox2-IRES-Puro

| (SEQ ID NO: 39) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | cccagtgtgg tggtacggga attccccggg ccccccaaag tcccggccgg gccgagggtc |
| 1981 | ggcggccgcc ggcgggccgg gcccgcgcac agcgcccgca tgtacaacat gatggagacg |
| 2041 | gagctgaagc cgccgggccc gcagcaaact tcggggggcg gcggcggcaa ctccaccgcg |
| 2101 | gcggcggccg gcggcaacca gaaaaacagc ccggaccgcg tcaagcggcc catgaatgcc |
| 2161 | ttcatggtgt ggtcccgcgg gcagcggcgc aagatggccc aggagaaccc caagatgcac |
| 2221 | aactcggaga tcagcaagcg cctgggcgcc gagtggaaac ttttgtcgga gacggagaag |
| 2281 | cggccgttca tcgacgaggc taagcggctg cgagcgctgc acatgaagga gcacccggat |
| 2341 | tataaatacc ggccccggcg gaaaaccaag acgctcatga agaaggataa gtacacgctg |
| 2401 | cccggcgggc tgctggcccc cggcggcaat agcatggcga gcggggtcgg ggtgggcgcc |
| 2461 | ggcctgggcg cgggcgtgaa ccagcgcatg gacagttacg cgcacatgaa cggctggagc |
| 2521 | aacggcagct acagcatgat gcaggaccag ctgggctacc cgcagcaccc gggcctcaat |
| 2581 | gcgcacggcg cagcgcagat gcagcccatg caccgctacg acgtgagcgc cctgcagtac |
| 2641 | aactccatga ccagctcgca gacctacatg aacggctcgc ccacctacag catgtcctac |
| 2701 | tcgcagcagg gcacccctgg catggctctt ggctccatgg gttcggtggt caagtccgag |
| 2761 | gccagctcca gcccccctgt ggttacctct tcctcccact ccagggcgcc ctgccaggcc |
| 2821 | ggggacctcc gggacatgat cagcatgtat ctccccggcg ccgaggtgcc ggaacccgcc |
| 2881 | gcccccagca gacttcacat gtcccagcac taccagagcg gcccggtgcc cggcacggcc |
| 2941 | attaacggca cactgcccct ctcacacatg tgagggccgg acagcgaact ggagggggga |
| 3001 | gaaattttca aagaaaaacg agggaaatgg gaggggtgca aaagaggaga gtaagaaaca |
| 3061 | gcatggagaa aacccggtac gctcaaaaaa aaaaaaaaaa aaaaaaaaaa aaaaaactcg |
| 3121 | agggccggcg cgccgcggcc gctacgtaaa ttccgcccct ctccctcccc cccccctaac |
| 3181 | gttactggcc gaagccgctt ggaataaggc cggtgtgcgt ttgtctatat gttattttcc |
| 3241 | accatattgc cgtcttttgg caatgtgagg gcccggaaac ctggccctgt cttcttgacg |
| 3301 | agcattccta ggggtctttc ccctctcgcc aaaggaatgc aaggtctgtt gaatgtcgtg |
| 3361 | aaggaagcag ttcctctgga agcttcttga agacaaacaa cgtctgtagc gaccctttgc |
| 3421 | aggcagcgga accccccacc tggcgacagg tgcctctgcg gccaaaagcc acgtgtataa |
| 3481 | gatacacctg caaaggcggc acaaccccag tgccacgttg tgagttggat agttgtggaa |
| 3541 | agagtcaaat ggctctcctc aagcgtattc aacaaggggc tgaaggatgc ccagaaggta |
| 3601 | ccccattgta tgggatctga tctggggcct cggtgcacat gctttacatg tgtttagtcg |
| 3661 | aggttaaaaa aacgtctagg ccccccgaac cacggggacg tggttttcct ttgaaaaaca |
| 3721 | cgatgataat atggccacaa ccatggttac cgagtacaag cccacggtgc gcctcgccac |
| 3781 | ccgcgacgac gtccccaggg ccgtacgcac cctcgccgcc gcgttcgccg actaccccgc |
| 3841 | cacgcgccac accgtcgatc cggaccgcca catcgagcgg gtcaccgagc tgcaagaact |
| 3901 | cttcctcacg cgcgtcgggc tcgacatcgg caaggtgtgg gtcgcggacg acggcgccgc |
| 3961 | ggtggcggtc tggaccacgc cggagagcgt cgaagcgggg gcggtgttcg ccgagatcgg |
| 4021 | cccgcgcatg gccgagttga gcggttcccg gctggccgcg cagcaacaga tggaaggcct |
| 4081 | cctggcgccg caccggccca aggagcccgc gtggttcctg gccaccgtcg gcgtctcgcc |
| 4141 | cgaccaccag ggcaagggtc tgggcagcgc cgtcgtgctc cccggagtgg aggcggccga |
| 4201 | gcgcgccggg gtgcccgcct tcctggagac ctccgcgccc cgcaacctcc ccttctacga |
| 4261 | gcggctcggc ttcaccgtca ccgccgacgt cgaggtgccc gaaggaccgc gcacctggtg |
| 4321 | catgacccgc aagcccggtg cctgagtcga cgataaaata aaagatttta tttagtctcc |
| 4381 | agaaaaaggg gggaatgaaa gaccccacct gtaggtttgg caagctagct taagtaacgc |
| 4441 | cattttgcaa ggcatggaaa aatacataac tgagaataga gaagttcaga tcaaggtcag |
| 4501 | gaacagatgg aacagctgaa tatgggccaa acaggatatc tgtggtaagc agttcctgcc |
| 4561 | ccggctcagg gccaagaaca gatggaacag ctgaatatgg gccaaacagg atatctgtgg |
| 4621 | taagcagttc ctgccccggc tcagggccaa gaacagatgg tccccagatg cggtccagcc |
| 4681 | ctcagcagtt tctagagaac catcagatgt ttccagggtg ccccaaggac ctgaaatgac |
| 4741 | cctgtgcctt atttgaacta accaatcagt tcgcttctcg cttctgttcg cgcgcttctg |
| 4801 | ctccccgagc tcaataaaag agcccacaac ccctcactcg gggcgccagt cctccgattg |
| 4861 | actgagtcgc ccgggtaccc gtgtatccaa taaaccctct tgcagttgca tccgacttgt |
| 4921 | ggtctcgctg ttccttggga gggtctcctc tgagtgattg actacccgtc agcgggggtc |
| 4981 | tttcacatgc agcatgtatc aaaattaatt tggttttttt tcttaagtat ttacattaaa |
| 5041 | tggccatagt tgcattaatg aatcggccaa cgcgcgggga gaggcggttt gcgtattggg |
| 5101 | cgctcttccg cttcctcgct cactgactcg ctgcgctcgg tcgttcggct gcggcgagcg |
| 5161 | gtatcagctc actcaaaggc ggtaatacgg ttatccacag aatcagggga taacgcagga |
| 5221 | aagaacatgt gagcaaaagg ccagcaaaag gccaggaacc gtaaaaaggc cgcgttgctg |
| 5281 | gcgtttttcc ataggctccg cccccctgac gagcatcaca aaaatcgacg ctcaagtcag |
| 5341 | aggtggcgaa acccgacagg actataaaga taccaggcgt ttccccctgg aagctccctc |
| 5401 | gtgcgctctc ctgttccgac cctgccgctt accggatacc tgtccgcctt tctcccttcg |
| 5461 | ggaagcgtgg cgctttctca tagctcacgc tgtaggtatc tcagttcggt gtaggtcgtt |
| 5521 | cgctccaagc tgggctgtgt gcacgaaccc cccgttcagc ccgaccgctg cgccttatcc |
| 5581 | ggtaactatc gtcttgagtc caacccggta agacacgact tatcgccact ggcagcagcc |
| 5641 | actggtaaca ggattagcag agcgaggtat gtaggcggtg ctacagagtt cttgaagtgg |
| 5701 | tggcctaact acggctacac tagaagaaca gtatttggta tctgcgctct gctgaagcca |
| 5761 | gttaccttcg gaaaaagagt tggtagctct tgatccggca aacaaaccac cgctggtagc |
| 5821 | ggtggttttt ttgtttgcaa gcagcagatt acgcgcagaa aaaaaggatc tcaagaagat |
| 5881 | cctttgatct tttctacggg gtctgacgct cagtggaacg aaaactcacg ttaagggatt |
| 5941 | ttggtcatga gattatcaaa aaggatcttc acctagatcc ttttgcggcc ggccgcaaat |
| 6001 | caatctaaag tatatatgag taaacttggt ctgacagtta ccaatgctta atcagtgagg |
| 6061 | cacctatctc agcgatctgt ctatttcgtt catccatagt tgcctgactc cccgtcgtgt |
| 6121 | agataactac gatacgggag ggcttaccat ctggccccag tgctgcaatg ataccgcgag |
| 6181 | acccacgctc accggctcca gatttatcag caataaacca gccagccgga agggccgagc |
| 6241 | gcagaagtgg tcctgcaact ttatccgcct ccatccagtc tattaattgt tgccgggaag |
| 6301 | ctagagtaag tagttcgcca gttaatagtt tgcgcaacgt tgttgccatt gctacaggca |
| 6361 | tcgtggtgtc acgctcgtcg tttggtatgg cttcattcag ctccggttcc caacgatcaa |
| 6421 | ggcgagttac atgatccccc atgttgtgca aaaaagcggt tagctccttc ggtcctccga |
| 6481 | tcgttgtcag aagtaagttg gccgcagtgt tatcactcat ggttatggca gcactgcata |
| 6541 | attctcttac tgtcatgcca tccgtaagat gcttttctgt gactggtgag tactcaacca |
| 6601 | agtcattctg agaatagtgt atgcggcgac cgagttgctc ttgcccggcg tcaatacggg |
| 6661 | ataataccgc gccacatagc agaactttaa aagtgctcat cattggaaaa cgttcttcgg |
| 6721 | ggcgaaaact ctcaaggatc ttaccgctgt tgagatccag ttcgatgtaa cccactcgtg |
| 6781 | cacccaactg atcttcagca tcttttactt tcaccagcgt ttctgggtga gcaaaaacag |
| 6841 | gaaggcaaaa tgccgcaaaa aagggaataa gggcgacacg gaaatgttga atactcatac |
| 6901 | tcttcctttt tcaatattat tgaagcattt atcagggtta ttgtctcatg agcggataca |
| 6961 | tatttgaatg tatttagaaa aataaacaaa taggggttcc gcgcacattt c |

pMXs-Klf4-IRES-Neo

| (SEQ ID NO: 40) | |
|---|---|
| 1 | cccgaaaagt gccacctgca taatgaaaga ccccacctgt aggtttggca agctagctta |
| 61 | agtaacgcca ttttgcaagg catggaaaaa tacataactg agaatagaaa agttcagatc |
| 121 | aaggtcagga acagatggaa cagctgaata tgggccaaac aggatatctg tggtaagcag |
| 181 | ttcctgcccc ggctcagggc caagaacaga tggaacagct gaatatgggc caaacaggat |
| 241 | atctgtggta agcagttcct gccccggctc agggccaaga acagatggtc cccagatgcg |
| 301 | gtccagccct cagcagtttc tagagaacca tcagatgttt ccagggtgcc ccaaggacct |
| 361 | gaaatgaccc tgtgccttat ttgaactaac caatcagttc gcttctcgct tctgttcgcg |
| 421 | cgcttctgct ccccgagctc aataaaagag cccacaaccc ctcactcggc gcgccagtcc |
| 481 | tccgattgac tgagtcgccc gggtacccgt gtatccaata aaccctcttg cagttgcatc |
| 541 | cgacttgtgg tctcgctgtt ccttgggagg gtctcctctg agtgattgac tacccgtcag |
| 601 | cgggggtctt tcatttgggg gctcgtccgg gatcgggaga cccctgccca gggaccaccg |
| 661 | acccaccacc gggaggtaag ctggccagca acttatctgt gtctgtccga ttgtctagtg |
| 721 | tctatgactg attttatgcg cctgcgtcgg tactagttag ctaactagct ctgtatctgg |
| 781 | cggacccgtg gtggaactga cgagttcgga acacccggcc gcaaccctgg gagacgtccc |
| 841 | agggacttcg ggggccgttt ttgtggcccg acctgagtcc aaaaatcccg atcgttttgg |
| 901 | actctttggt gcacccccct aataggaggg atatgtggtt ctggtaggag acgagaacct |
| 961 | aaaacagttc ccgcctccgt ctgaattttt gctttcggtt tgggaccgaa gccgcgccgc |
| 1021 | gcgtcttgtc tgctgcagca tcgttctgtg ttgtctctgt ctgactgtgt ttctgtattt |
| 1081 | gtctgaaaat tagggccaga ctgttaccac tcccttaagt ttgaccttag gtcactggaa |
| 1141 | agatgtcgag cggatcgctc acaaccagtc ggtagatgtc aagaagagac gttgggttac |
| 1201 | cttctgctct gcagaatggc caacctttaa cgtcggatgg ccgcgagacg gcacctttaa |
| 1261 | ccgagacctc atcacccagg ttaagatcaa ggtcttttca cctggcccgc atggacaccc |
| 1321 | agaccaggtc ccctacatcg tgacctggga agccttggct tttgaccccc ctccctgggt |
| 1381 | caagcccttt gtacacccta agcctccgcc tcctcttcct ccatccgccc cgtctctccc |
| 1441 | ccttgaacct cctcgttcga ccccgcctcg atcctccctt tatccagccc tcactccttc |
| 1501 | tctaggcgcc cccatatggc catatgagat cttatatggg gcacccccgc cccttgtaaa |
| 1561 | cttccctgac cctgacatga caagagttac taacagcccc tctctccaag ctcacttaca |
| 1621 | ggctctctac ttagtccagc acgaagtctg gagacctctg gcggcagcct accaagaaca |
| 1681 | actggaccga ccggtggtac ctcaccctta ccgagtcggc gacacagtgt gggtccgccg |
| 1741 | acaccagact aagaacctag aacctcgctg gaaaggacct tacacagtcc tgctgaccac |
| 1801 | ccccaccgcc ctcaaagtag acggcatcgc agcttggata cacgccgccc acgtgaaggc |
| 1861 | tgccgacccc gggggtggac catcctctag actgccggat ctagctagtt aattaaggat |
| 1921 | cccagtgtgg tggtacggga attctcgagg cgaccgcgac agtggtgggg gacgctgctg |
| 1981 | agtggaagag agcgcagccc ggccaccgga cctacttact cgccttgctg attgtctatt |
| 2041 | tttgcgttta caacttttct aagaactttt gtatacaaag gaacttttta aaaaagacgc |
| 2101 | ttccaagtta tatttaatcc aaagaagaag gatctcggcc aatttggggt tttgggtttt |
| 2161 | ggcttcgttt cttctcttcg ttgactttgg ggttcaggtg ccccagctgc ttcgggctgc |
| 2221 | cgaggacctt ctgggccccc acattaatga ggcagccacc tggcgagtct gacatggctg |
| 2281 | tcagcgacgc gctgctccca tctttctcca cgttcgcgtc tggcccggcg ggaagggaga |
| 2341 | agacactgcg tcaagcaggt gccccgaata accgctggcg ggaggagctc tcccacatga |
| 2401 | agcgacttcc cccagtgctt cccggccgcc cctatgacct ggcggcggcg accgtggcca |
| 2461 | cagacctgga gagcggcgga gccggtgcgg cttgcggcgg tagcaacctg gcgcccctac |
| 2521 | ctcggagaga gaccgaggag ttcaacgatc tcctggacct ggactttatt ctctccaatt |
| 2581 | cgctgaccca tcctccggag tcagtggccg ccaccgtgtc ctcgtcagcg tcagcctcct |
| 2641 | cttcgtcgtc gccgtcgagc agcggccctg ccagcgcgcc ctccacctgc agcttcacct |
| 2701 | atccgatccg ggccgggaac gacccgggcg tggcgccggg cggcacgggc ggaggcctcc |
| 2761 | tctatggcag ggagtccgct ccccctccga cggctccctt caacctggcg gacatcaacg |
| 2821 | acgtgagccc ctcgggcggc ttcgtggccg agctcctgcg gccagaattg gacccggtgt |
| 2881 | acattccgcc gcagcagccg cagccgccag gtggcgggct gatgggcaag ttcgtgctga |
| 2941 | aggcgtcgct gagcgcccct ggcagcgagt acggcagccc gtcggtcatc agcgtcagca |
| 3001 | aaggcagccc tgacggcagc cacccggtgg tggtggcgcc ctacaacggc gggccgccgc |
| 3061 | gcacgtgccc caagatcaag caggaggcgg tctcttcgtg cacccacttg ggcgctggac |
| 3121 | cccctctcag caatggccac cggccggctg cacacgactt ccccctgggg cggcagctcc |
| 3181 | ccagcaggac taccccgacc ctgggtcttg aggaagtgct gagcagcagg gactgtcacc |
| 3241 | ctgccctgcc gcttcctccc ggcttccatc cccacccggg gcccaattac ccatccttcc |
| 3301 | tgcccgatca gatgcagccg caagtcccgc cgctccatta ccaagagctc atgccacccg |
| 3361 | gttcctgcat gccagaggag cccaagccaa agaggggaag acgatcgtgg ccccggaaaa |
| 3421 | ggaccgccac ccacacttgt gattacgcgg gctgcggcaa aacctacaca aagagttccc |
| 3481 | atctcaaggc acacctgcga acccacacag gtgagaaacc ttaccactgt gactgggacg |
| 3541 | gctgtggatg gaaattcgcc cgctcagatg aactgaccag gcactaccgt aaacacacgg |
| 3601 | ggcaccgccc gttccagtgc caaaaatgcg accgagcatt ttccaggtcg gaccacctcg |
| 3661 | ccttacacat gaagaggcat ttttaaatcc cagacagtgg atatgaccca cactgccaga |
| 3721 | agagaattcc tgcaggcctc gagggccggc gcgccgcggc cgctacgtaa attccgcccc |
| 3781 | tctccctccc ccccccctaa cgttactggc cgaagccgct tggaataagg ccggtgtgcg |
| 3841 | tttgtctata tgttattttc caccatattg ccgtcttttg gcaatgtgag ggcccggaaa |
| 3901 | cctggccctg tcttcttgac gagcattcct aggggtcttt cccctctcgc caaaggaatg |
| 3961 | caaggtctgt tgaatgtcgt gaaggaagca gttcctctgg aagcttcttg aagacaaaca |
| 4021 | acgtctgtag cgaccctttg caggcagcgg aaccccccac ctggcgacag gtgcctctgc |
| 4081 | ggccaaaagc cacgtgtata agatacacct gcaaaggcgg cacaacccca gtgccacgtt |
| 4141 | gtgagttgga tagttgtgga aagagtcaaa tggctctcct caagcgtatt caacaagggg |
| 4201 | ctgaaggatg cccagaaggt accccattgt atgggatctg atctggggcc tcggtgcaca |
| 4261 | tgctttacat gtgtttagtc gaggttaaaa aaacgtctag gccccccgaa ccacggggac |
| 4321 | gtggttttcc tttgaaaaac acgatgataa tatggccaca accatggtta ttgaacaaga |
| 4381 | tggattgcac gcaggttctc cggccgcttg ggtggagagg ctattcggct atgactgggc |
| 4441 | acaacagaca atcggctgct ctgatgccgc cgtgttccgg ctgtcagcgc aggggcgccc |
| 4501 | ggttcttttt gtcaagaccg acctgtccgg tgccctgaat gaactgcagg acgaggcagc |
| 4561 | gcggctatcg tggctggcca cgacgggcgt tccttgcgca gctgtgctcg acgttgtcac |
| 4621 | tgaagcggga agggactggc tgctattggg cgaagtgccg gggcaggatc tcctgtcatc |
| 4681 | tcaccttgct cctgccgaga aagtatccat catggctgat gcaatgcggc ggctgcatac |
| 4741 | gcttgatccg gctacctgcc cattcgacca ccaagcgaaa catcgcatcg agcgagcacg |
| 4801 | tactcggatg gaagccggtc ttgtcgatca ggatgatctg gacgaagagc atcaggggct |
| 4861 | cgcgccagcc gaactgttcg ccaggctcaa ggcgcgcatg cccgacggcg aggatctcgt |
| 4921 | cgtgacccat ggcgatgcct gcttgccgaa tatcatggtg gaaaatggcc gcttttctgg |
| 4981 | attcatcgac tgtggccggc tgggtgtggc ggaccgctat caggacatag cgttggctac |
| 5041 | ccgtgatatt gctgaagagc ttggcggcga atgggctgac cgcttcctcg tgctttacgg |
| 5101 | tatcgccgct cccgattcgc agcgcatcgc cttctatcgc cttcttgacg agttcttctg |
| 5161 | agtcgacgat aaaataaaag attttattta gtctccagaa aaagggggga atgaaagacc |
| 5221 | ccacctgtag gtttggcaag ctagcttaag taacgccatt ttgcaaggca tggaaaaata |
| 5281 | cataactgag aatagagaag ttcagatcaa ggtcaggaac agatggaaca gctgaatatg |
| 5341 | ggccaaacag gatatctgtg gtaagcagtt cctgccccgg ctcagggcca agaacagatg |
| 5401 | gaacagctga atatgggcca aacaggatat ctgtggtaag cagttcctgc cccggctcag |
| 5461 | ggccaagaac agatggtccc cagatgcggt ccagccctca gcagtttcta gagaaccatc |
| 5521 | agatgtttcc agggtgcccc aaggacctga aatgaccctg tgccttattt gaactaacca |
| 5581 | atcagttcgc ttctcgcttc tgttcgcgcg cttctgctcc ccgagctcaa taaaagagcc |
| 5641 | cacaacccct cactcggggc gccagtcctc cgattgactg agtcgcccgg gtacccgtgt |
| 5701 | atccaataaa ccctcttgca gttgcatccg acttgtggtc tcgctgttcc ttgggagggt |
| 5761 | ctcctctgag tgattgacta cccgtcagcg ggggtctttc acatgcagca tgtatcaaaa |
| 5821 | ttaatttggt tttttttctt aagtatttac attaaatggc catagttgca ttaatgaatc |
| 5881 | ggccaacgcg cggggagagg cggtttgcgt attgggcgct cttccgcttc ctcgctcact |
| 5941 | gactcgctgc gctcggtcgt tcggctgcgg cgagcggtat cagctcactc aaaggcggta |
| 6001 | atacggttat ccacagaatc aggggataac gcaggaaaga acatgtgagc aaaaggccag |
| 6061 | caaaaggcca ggaaccgtaa aaaggccgcg ttgctggcgt ttttccatag gctccgcccc |
| 6121 | cctgacgagc atcacaaaaa tcgacgctca agtcagaggt ggcgaaaccc gacaggacta |
| 6181 | taaagatacc aggcgtttcc ccctggaagc tccctcgtgc gctctcctgt tccgaccctg |
| 6241 | ccgcttaccg gatacctgtc cgcctttctc ccttcgggaa gcgtggcgct ttctcatagc |
| 6301 | tcacgctgta ggtatctcag ttcggtgtag gtcgttcgct ccaagctggg ctgtgtgcac |
| 6361 | gaaccccccg ttcagcccga ccgctgcgcc ttatccggta actatcgtct tgagtccaac |
| 6421 | ccggtaagac acgacttatc gccactggca gcagccactg gtaacaggat tagcagagcg |
| 6481 | aggtatgtag gcggtgctac agagttcttg aagtggtggc ctaactacgg ctacactaga |
| 6541 | agaacagtat ttggtatctg cgctctgctg aagccagtta ccttcggaaa aagagttggt |
| 6601 | agctcttgat ccggcaaaca aaccaccgct ggtagcggtg gtttttttgt ttgcaagcag |
| 6661 | cagattacgc gcagaaaaaa aggatctcaa gaagatcctt tgatcttttc tacggggtct |
| 6721 | gacgctcagt ggaacgaaaa ctcacgttaa gggattttgg tcatgagatt atcaaaaagg |
| 6781 | atcttcacct agatcctttt gcggccggcc gcaaatcaat ctaaagtata tatgagtaaa |
| 6841 | cttggtctga cagttaccaa tgcttaatca gtgaggcacc tatctcagcg atctgtctat |
| 6901 | ttcgttcatc catagttgcc tgactccccg tcgtgtagat aactacgata cgggagggct |
| 6961 | taccatctgg ccccagtgct gcaatgatac cgcgagaccc acgctcaccg gctccagatt |
| 7021 | tatcagcaat aaaccagcca gccggaaggg ccgagcgcag aagtggtcct gcaactttat |
| 7081 | ccgcctccat ccagtctatt aattgttgcc gggaagctag agtaagtagt tcgccagtta |
| 7141 | atagtttgcg caacgttgtt gccattgcta caggcatcgt ggtgtcacgc tcgtcgtttg |
| 7201 | gtatggcttc attcagctcc ggttcccaac gatcaaggcg agttacatga tcccccatgt |
| 7261 | tgtgcaaaaa agcggttagc tccttcggtc ctccgatcgt tgtcagaagt aagttggccg |
| 7321 | cagtgttatc actcatggtt atggcagcac tgcataattc tcttactgtc atgccatccg |
| 7381 | taagatgctt ttctgtgact ggtgagtact caaccaagtc attctgagaa tagtgtatgc |
| 7441 | ggcgaccgag ttgctcttgc ccggcgtcaa tacgggataa taccgcgcca catagcagaa |
| 7501 | ctttaaaagt gctcatcatt ggaaaacgtt cttcggggcg aaaactctca aggatcttac |
| 7561 | cgctgttgag atccagttcg atgtaaccca ctcgtgcacc caactgatct tcagcatctt |
| 7621 | ttactttcac cagcgtttct gggtgagcaa aaacaggaag gcaaaatgcc gcaaaaaagg |
| 7681 | gaataagggc gacacggaaa tgttgaatac tcatactctt cctttttcaa tattattgaa |
| 7741 | gcatttatca gggttattgt ctcatgagcg gatacatatt tgaatgtatt tagaaaaata |
| 7801 | aacaaatagg ggttccgcgc acatttc |

While preferred embodiments have been described herein, such embodiments are provided by way of example only. Numerous variations, changes, and substitutions are feasible. It should be understood that various alternatives to the embodiments of the methods and compositions described herein may be employed in practicing the invention. It is intended that the following claims define the scope of the invention and that methods and compositions within the scope of these claims and their equivalents be covered thereby.

1-44. (canceled)

45. A method for identifying induced pluripotent stem cells, comprising introducing into the genome of a plurality of mammalian cells an expression vector encoding a selection marker operably linked to a promoter that drives expression of the encoded selection marker in the plurality of mammalian cells and is capable of undergoing transcriptional silencing in a pluripotent stem cell, and culturing the plurality of mammalian cells for a period after the introducing step, wherein
(i) the plurality of mammalian cells comprise at least one exogenous nucleic acid encoding at least one induction factor;
(ii) the plurality of mammalian cells comprise at least one exogenous induction factor;
(iii) the plurality of mammalian cells are contacted with at least one inducing agent or
(iv) any combination of (i)-(iii); and
assaying activity or expression of the encoded selection marker from the introduced expression vector, wherein reduced or absent expression of the selection marker in one or more of the plurality of mammalian cells after the period indicates that the one or more mammalian cells are induced pluripotent stem cells.

46. The method of claim 45, wherein the promoter comprises one or more retroviral silencing elements.

47. The method of claim 45, wherein the introducing step comprises transducing the cell with a recombinant retrovirus comprising the expression vector for expression of the selection marker.

48. The method of claim 45, wherein the at least one nucleic acid encodes three exogenous induction factors.

49. The method of claim 45, wherein the at least one nucleic acid encodes four exogenous induction factors.

50. The method of claim 45, wherein the selection marker comprises the amino acid sequence of an enzyme or a fluorescent protein.

51. The method of claim 50, wherein the enzyme comprises the amino acid sequence of a HSV thymidine kinase, of a β-lactamase, or a luciferase.

52. The method of claim 50, wherein the selection marker comprises the amino acid sequence of a fluorescent protein.

53. The method of claim 52, wherein the fluorescent protein comprises the amino acid sequence of a fluorescent timer or a photoactivatible fluorescent protein.

54. The method of claim 50, wherein the selection marker comprises the amino acid sequence of an enzyme and a fluorescent protein.

55. A method for inducing differentiation of induced pluripotent stem cells, comprising depleting a population of induced pluripotent stem cells of cells that express at least one selection marker polypeptide, wherein expression of the selection marker polypeptide is driven by a promoter that undergoes transcriptional silencing in pluripotent stem cells, and differentiating the depleted population.

56. The method of claim 55, wherein the selection marker polypeptide comprises the amino acid sequence of an enzyme.

57. The method of claim 56, wherein the selection marker polypeptide comprises the amino acid sequence of a HSV-thymidine kinase a β-lactamase, or a luciferase.

58. The method of claim 55, wherein the selection marker polypeptide comprises the amino acid sequence of a fluorescent protein.

59. The method of claim 58, wherein the fluorescent reporter protein comprises the amino acid sequence of a fluorescent timer protein, a photoactivatible fluorescent protein, or EGFP.

60. A recombinant mammalian cell comprising one or more exogenous nucleic acids encoding:
(i) an induction factor; and
(ii) a selection marker polypeptide comprising the amino acid sequence of a β-lactamase, a fluorescent timer, a photoactivatible fluorescent protein, HSV-thymidine kinase, or a luciferase; wherein
expression of the encoded induction factor and the encoded selection marker is driven by a promoter comprising a retroviral silencing element, and wherein the exogenous nucleic acid encoding the selection marker is integrated in the genome of the recombinant mammalian cell.

61. The recombinant mammalian cell of claim 60, wherein the promoter comprises the MoMLV LTR promoter.

62. The recombinant mammalian cell of claim 60, wherein the one or more exogenous nucleic acids encode (i) Oct4, Klf4, Sox2, and c-Myc, or (ii) Oct4, Klf4, and Sox2, or functional homologs thereof.

63. A nucleic acid comprising:
(i) a promoter comprising one or more retroviral silencing elements;
(ii) an open reading frame encoding an induction factor; and
(iii) an open reading frame encoding a fluorescent timer protein, a photoactivatable fluorescent protein, a β-lactamase, a luciferase, or a HSV-thymidine kinase, wherein
(i) is operably connected to (ii) or (iii); and (ii) and (iii) are operably linked to each other.

64. The nucleic acid of claim 63, wherein (iii) is an open reading frame encoding both a fluorescent timer protein and a HSV-thymidine kinase.


SEQUENCE LISTING (41 sequences)

SEQ ID NO: 1 - DNA, 780 bp, Moloney murine leukemia virus
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctgg

SEQ ID NO: 2 - DNA, 56 bp, Moloney murine leukemia virus
ccccacctgtaggtttggcaagctagcttaagtaacgccattttgcaaggcatgga

SEQ ID NO: 3 - DNA, 92 bp, Moloney murine leukemia virus
aaggtcaggaacagatggaacagctgaatatgggccaaacaggatatctgtggtaagcagttcctgccccggctcagggccaagaacagatg

SEQ ID NO: 4 - DNA, 33 bp, Moloney murine leukemia virus
cgggggtctttcatttgggggctcgtccgggat

SEQ ID NO: 5 - DNA, 199 bp, Moloney murine leukemia virus
gttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactaccc

SEQ ID NO: 6 - protein, 304 aa, Herpes simplex virus
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAASPRLWAACQYGAVSAGRRVVAGGLGTAFGGGRAAPGCRAPEQRGPTTPYRGHVIYPVSGPRVAGPQRRPV

SEQ ID NO: 7 - protein, 21 aa, Unknown
  Description of Unknown Exemplary 2A peptide sequence
GSGEGRGSLLTCGDVEFNPGP

SEQ ID NO: 8 - DNA, 63 bp, Unknown
  Description of Unknown Exemplary 2A oligonucleotide sequence
ggcagtggagagggcagaggaagtctgctaacatgcggtgacgtcgaggagaatcctggccca

SEQ ID NO: 9 - protein, 21 aa, Unknown
  Description of Unknown Exemplary 2A peptide sequence
GSGEGRGSLLTCGDVEENPGP

SEQ ID NO: 10 - DNA, 75 bp, Unknown
  Description of Unknown Exemplary 2A oligonucleotide sequence
ggttctggcgtgaaacagactttgaattttgaccttctcaagttggcgggagacgtggagtccaacccagggccc

SEQ ID NO: 11 - protein, 24 aa, Unknown
  Description of Unknown Exemplary 2A peptide sequence
GSGVKQTNFDLLKLAGDVESNPGP

SEQ ID NO: 12 - protein, 11 aa, Unknown
  Description of Unknown Exemplary protein transduction domain peptide sequence
YGRKKRRQRRR

SEQ ID NO: 13 - protein, 8 aa, Unknown
  Description of Unknown Exemplary protein transduction domain peptide sequence
RKKRRQRR

SEQ ID NO: 14 - protein, 11 aa, Unknown
  Description of Unknown Exemplary protein transduction domain peptide sequence
YARAAARQARA

SEQ ID NO: 15 - protein, 11 aa, Unknown
  Description of Unknown Exemplary protein transduction domain peptide sequence
THRLPRRRRRR

SEQ ID NO: 16 - protein, 11 aa, Unknown
  Description of Unknown Exemplary protein transduction domain peptide sequence
GGRRARRRRRR

SEQ ID NO: 17 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 18 - protein, 153 aa, Homo sapiens
DIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSS

SEQ ID NO: 19 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 20 - protein, 76 aa, Homo sapiens
RVKRPMNAFMVWSRGQRRKMAQENPKMHNSEISKRLGAEWKLLSETEKRPFIDEAKRLRALHMKEHPDYKYRPRRK

SEQ ID NO: 21 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 22 - protein, 88 aa, Homo sapiens
KRTATHTCDYAGCGKTYTKSSHLKAHLRTHTGEKPYHCDWDGCGWKFARSDELTRHYRKHTGHRPFQCQKCDRAFSRSDHLALHMKRH

SEQ ID NO: 23 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 24 - protein, 85 aa, Homo sapiens
KRRTHNVLERQRRNELKRSFFALRDQIPELENNEKAPKVVILKKATAYILSVQAEEQKLISEEDLLRKRREQLKHKLEQLRNSCA

SEQ ID NO: 25 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 26 - protein, 209 aa, Mus musculus
MGSVSNQQFAGGCAKAAEKAPEEAPPDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKNMQKRRSKGDRCYNCGGLDHHAKECKLPPQPKKCHFCQSINHMVASCPLKAQQGPSSQGKPAYFREEEEEIHSPALLPEAQN

SEQ ID NO: 27 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 28 - protein, 305 aa, Mus musculus
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 29 - protein, 549 aa, Artificial Sequence
  Description of Artificial Sequence Synthetic polypeptide
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAASPRLWAACQYGAVSAGRRVVAGGLGTAFGGGRAAPGCRAPEQRGPTTPYRGHVIYPVSGPRVAGPQRRPVPGSIATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - DNA, 1650 bp, Artificial Sequence
  Description of Artificial Sequence Synthetic polynucleotide
atggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgtcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtacctggttctattgctactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 31 - protein, 536 aa, Artificial Sequence
  Description of Artificial Sequence Synthetic polypeptide
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAASPRLWAACQYGAVSAGRRVVAGGLGTAFGGGRAAPGCRAPEQRGPTTPYRGHVIYPVSGPRVAGPQRRPVPGSIATMVRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGEIHKALKLKDGGHYLVEFKSIYMAKKPVQLPGYYYVDTKLDITSHNEDYTIVEQYERTEGRHHLFL

SEQ ID NO: 32 - DNA, 1611 bp, Artificial Sequence
  Description of Artificial Sequence Synthetic polynucleotide
atggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgtcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtacctggttctattgctactatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggacaccaagctggacatcacctcccacaacgaggactacaccatcgtggagcagtacgagcgcaccgagggccgccaccacctgttcctgtag

SEQ ID NO: 33 - DNA, 7987 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattcccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaagaattcgcggcaattcctgcaggcctcgagggccggcgcgccgcggccgcgactctagaatttcgacctcgacattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgtcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggctcgacggtacctttaagaccaatgacttacaaggcagctgtagatcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 34 - DNA, 7946 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattccccgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaaaaaaaaaaaaaaaaaaaaaaaaaaaaactcgagggccggcgcgccgcggccgcgactctagaatttcgacctcgacattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgtcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggctcgacggtacctttaagaccaatgacttacaaggcagctgtagatcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 35 - DNA, 8567 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattctcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcctgcaggcctcgagggccggcgcgccgcggccgcgactctagaatttcgacctcgacattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgtcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggctcgacggtacctttaagaccaatgacttacaaggcagctgtagatcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 36 - DNA, 8135 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaatcgacggtatcgataagcttgatatctgcggcctagctagccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcggccgcgactctagaatttcgacctcgacattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgtcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggctcgacggtacctttaagaccaatgacttacaaggcagctgtagatcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 37 - DNA, 5964 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattcctgcaggcctcgagggccggcgcgccgcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 38 - DNA, 7442 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatccgaattcccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaagaattcgcggcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgtatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaacccgtcgcggagctcatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgagtcgagaattcggtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 39 - DNA, 7011 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattccccgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaaaaaaaaaaaaaaaaaaaaaaaaaaaaactcgagggccggcgcgccgcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 40 - DNA, 7827 bp, Artificial Sequence
  Description of Artificial Sequence Synthetic polynucleotide
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcccagtgtggtggtacgggaattctcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcctgcaggcctcgagggccggcgcgccgcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 41 - DNA, 14 bp, Artificial Sequence
  Description of Artificial Sequence Synthetic oligonucleotide
cgaccacgtggtgc